SEQUENCE LISTING (72 sequences)

SEQ ID NO: 1 - protein, 16 aa, Artificial Sequence
  Core Peptide for Lasso 1
GNWHGTAPDWFFNYYW

SEQ ID NO: 2 - protein, 16 aa, Artificial Sequence
  Core Peptide for Lasso 2
  misc_feature (16)..(16): Xaa is 7-hydroxyl-trptophan
GNWHGTAPDWFFNYYX

SEQ ID NO: 3 - protein, 16 aa, Artificial Sequence
  Core Peptide for Lasso 3
GNWHGTSPDWFFNYYW

SEQ ID NO: 4 - protein, 16 aa, Artificial Sequence
  Core Peptide for Lasso 4
  misc_feature (16)..(16): Xaa is 7-hydroxyl-trptophan
GNWHGTSPDWFFNYYX

SEQ ID NO: 5 - protein, 15 aa, Artificial Sequence
  Core Peptide for Lasso 5
GNWHGTSPDWFFNYY

SEQ ID NO: 6 - protein, 17 aa, Artificial Sequence
  Core Peptide for Lasso 6
GNWHGTSPDWFFNYYWW

SEQ ID NO: 7 - protein, 16 aa, Artificial Sequence
  Core Peptide for Lasso 7
GNWHGTSPDWFFNYYA

SEQ ID NO: 8 - protein, 16 aa, Artificial Sequence
  Core Peptide for Lasso 8
GNWHGTSPDWFFNYYF

SEQ ID NO: 9 - protein, 16 aa, Artificial Sequence
  Core Peptide for Lasso 9
GNWHGTSPDWFFNYYY

SEQ ID NO: 10 - protein, 19 aa, Artificial Sequence
  Core Peptide for Lasso 10
GNWHGTSPDWFFNYYNYYW

SEQ ID NO: 11 - protein, 19 aa, Artificial Sequence
  Core Peptide for Lasso 11
GNWHGTSPDWFFNYYNIIW

SEQ ID NO: 12 - protein, 22 aa, Artificial Sequence
  Core Peptide for Lasso 12
GNWHGTSPDWFFNYYAHLDIIW

SEQ ID NO: 13 - protein, 16 aa, Artificial Sequence
  Core Peptide for Lasso 13
  misc_feature (16)..(16): Xaa = (2S)-2-amino-3-(1H-pyrrolo[5,4-b]pyridin- 3-yl)propanoic (Aza derivative of Tryptophan)
GNWHGTSPDWFFNYYX

SEQ ID NO: 14 - protein, 16 aa, Artificial Sequence
  Core Peptide for Lasso 14
  misc_feature (16)..(16): Xaa is Tryptophan having a C-terminal methyl ester group (-CO2Me) in place of the carboxylic acid group (-CO2H)
GNWHGTSPDWFFNYYX

SEQ ID NO: 15 - protein, 16 aa, Artificial Sequence
  Core Peptide for Lasso 15
  misc_feature (16)..(16): Xaa is Tryptophan having a C-terminal benzyl ester group (-CO2Bn) in place of the carboxylic acid group (-CO2H)
GNWHGTSPDWFFNYYX

SEQ ID NO: 16 - protein, 16 aa, Artificial Sequence
  Core Peptide for Lasso 16
  misc_feature (16)..(16): Xaa is Tryptophan having a C-terminal amide group (-CONH2) in place of the carboxylic acid group (-CO2H)
GNWHGTSPDWFFNYYX

SEQ ID NO: 17 - protein, 16 aa, Artificial Sequence
  Core Peptide for Lasso 17
  misc_feature (16)..(16): Xaa is 2-naphthylalanine
GNWHGTSPDWFFNYYX

SEQ ID NO: 18 - protein, 44 aa, Artificial Sequence
  Precursor peptide for Lasso 1
MTEQSEQTPAEYIPPMLVEVGEFTEDTLGNWHGTAPDWFFNYYW

SEQ ID NO: 19 - protein, 44 aa, Artificial Sequence
  Precursor peptide for Lasso 2
  misc_feature (44)..(44): Xaa is 7-hydroxyl-trptophan
MTEQSEQTPAEYIPPMLVEVGEFTEDTLGNWHGTAPDWFFNYYX

SEQ ID NO: 20 - protein, 44 aa, Artificial Sequence
  Precursor peptide for Lasso 3
MTEQSEQTPAEYIPPMLVEVGEFTEDTLGNWHGTSPDWFFNYYW

SEQ ID NO: 21 - protein, 44 aa, Artificial Sequence
  Precursor peptide for Lasso 4
  misc_feature (44)..(44): Xaa is 7-hydroxyl-trptophan
MTEQSEQTPAEYIPPMLVEVGEFTEDTLGNWHGTSPDWFFNYYX

SEQ ID NO: 22 - protein, 43 aa, Artificial Sequence
  Precursor peptide for Lasso 5
MTEQSEQTPAEYIPPMLVEVGEFTEDTLGNWHGTSPDWFFNYY

SEQ ID NO: 23 - protein, 45 aa, Artificial Sequence
  Precursor peptide for Lasso 6
MTEQSEQTPAEYIPPMLVEVGEFTEDTLGNWHGTSPDWFFNYYWW

SEQ ID NO: 24 - protein, 44 aa, Artificial Sequence
  Precursor peptide for Lasso 7
MTEQSEQTPAEYIPPMLVEVGEFTEDTLGNWHGTSPDWFFNYYA

SEQ ID NO: 25 - protein, 44 aa, Artificial Sequence
  Precursor peptide for Lasso 8
MTEQSEQTPAEYIPPMLVEVGEFTEDTLGNWHGTSPDWFFNYYF

SEQ ID NO: 26 - protein, 44 aa, Artificial Sequence
  Precursor peptide for Lasso 9
MTEQSEQTPAEYIPPMLVEVGEFTEDTLGNWHGTSPDWFFNYYY

SEQ ID NO: 27 - protein, 47 aa, Artificial Sequence
  Precursor peptide for Lasso 10
MTEQSEQTPAEYIPPMLVEVGEFTEDTLGNWHGTSPDWFFNYYNYYW

SEQ ID NO: 28 - protein, 47 aa, Artificial Sequence
  Precursor peptide for Lasso 11
MTEQSEQTPAEYIPPMLVEVGEFTEDTLGNWHGTSPDWFFNYYNIIW

SEQ ID NO: 29 - protein, 50 aa, Artificial Sequence
  Precursor peptide for Lasso 12
MTEQSEQTPAEYIPPMLVEVGEFTEDTLGNWHGTSPDWFFNYYAHLDIIW

SEQ ID NO: 30 - protein, 44 aa, Artificial Sequence; identical to SEQ ID NO: 21
  Precursor peptide for Lasso 13
  misc_feature (44)..(44): Xaa = (2S)-2-amino-3-(1H-pyrrolo[5,4-b]pyridin- 3-yl)propanoic (Aza derivative of Tryptophan)

SEQ ID NO: 31 - protein, 44 aa, Artificial Sequence; identical to SEQ ID NO: 21
  Precursor peptide for Lasso 14
  misc_feature (44)..(44): Xaa is Tryptophan having a C-terminal methyl ester group (-CO2Me) in place of the carboxylic acid group (-CO2H)

SEQ ID NO: 32 - protein, 44 aa, Artificial Sequence; identical to SEQ ID NO: 21
  Precursor peptide for Lasso 15
  misc_feature (44)..(44): Xaa is Tryptophan having a C-terminal benzyl ester group (-CO2Bn) in place of the carboxylic acid group (-CO2H)

SEQ ID NO: 33 - protein, 44 aa, Artificial Sequence; identical to SEQ ID NO: 21
  Precursor peptide for Lasso 16
  misc_feature (44)..(44): Tryptophan having a C-terminal amide group (-CONH2) in place of the carboxylic acid group (-CO2H)

SEQ ID NO: 34 - protein, 44 aa, Artificial Sequence; identical to SEQ ID NO: 21
  Precursor peptide for Lasso 17
  misc_feature (44)..(44): Xaa is 2-naphthylalanine

SEQ ID NO: 35 - protein, 145 aa, Artificial Sequence
  Lasso peptidase
MSQSMALDQRARLPLARRLLPLLAVGAARPLARLKPARLRAVLEFARRGAAPAGAGQAQRAREQVVSVSLRCAGQNCLQRSLATVLLCRARGVWPTWCTGVRTHPFAAHAWVEAEGRLIGEPHPEGYYKPLLTVPPRTGAGAGDR

SEQ ID NO: 36 - protein, 615 aa, Artificial Sequence
  Lasso cyclase
MAKLSAGFVVLPDTADGAEIRALVPFAPARVIAHASGRPWLVGEWSQGQVRVASAGPVRLAVSGTCPVTDDGLARIAARISRLSEVDRAVPALLGSCHIVASVDGHVRFQGSASGLRRVFHTRVNGVRVAADRSDVLAAMTGAGVDEETLALHVACGLQVPFPANARSAWSGISTLAPENCLLWEGDRDRETVWWRPPEPDRSLREGTAAVRDTLAAVVGRQAPVEGRLSADLSGGLDSTSLCFLAARHTPELLTFRWGEADAGNDDAVYAGQAARLLDRAEHLVVPQHELPDIFADPALAVSAEEPLSLTRATARIRRGARLLADRGSRRHLAGHGGDELFSPMPSYLHQLMRRHPVTALKHVRAHCALKRWPLKATLAALAQPGSLPAWWREQAGLLTEPRPPLRRPALGWGLGPVRAAPWMTPEGVELAREALRRTAEWAQPLAEDLATHTMVLMIRSNTASYRLLARLYAEAGVELDMPYLDDAVIDAVLRVPAHAHAGPWRYKPLLADAMHGIVPDGIRARSTKGEFGEDIRRGLRRNLPAILDLFADSELAARGLIDTGALRLRLGGPQPDNTTAQALESLLGCETWLRTTSGPGTIPRAGDGTVPSEV

SEQ ID NO: 37 - protein, 84 aa, Artificial Sequence
  Lasso RRE
MRLHPDVSMTDTDDGTVLLHQRTGRYWQLNVTGSRVLHRLLDGDTPETIADGLAAAHGIDPQRARQDVGAVIEQLRTAELTVAS

SEQ ID NO: 38 - DNA, 132 bp, Artificial Sequence
  Precursor Peptide for Lasso 3
atgaccgagcagtccgagcagacacccgcggaatacattccgccgatgctcgtcgaggtcggcgagttcaccgaagacacgttgggcaactggcacggcacatcgcccgactggttcttcaactactactgg

SEQ ID NO: 39 - DNA, 435 bp, Artificial Sequence
  Lasso peptidase
atgagccagtccatggcactggaccagcgggcccggctcccgcttgcgcgccgtctgctgccgctgctggccgtcggcgccgcccggccgctcgcccgcctcaagcccgcgcggctgcgcgccgtgctcgaattcgcccgccgtggtgcggcgccggcgggtgccgggcaggcgcagcgggcgcgcgagcaggtcgtctcggtgagcctgcgctgtgcggggcagaactgtctgcagcgctcgctcgccaccgtgctgctgtgccgggcccgcggggtgtggccgacctggtgcaccggcgtacggacgcacccgttcgccgcccacgcctgggttgaggccgagggccggctgatcggcgaaccgcaccccgagggctattacaagccgctgctgaccgtcccgccccgcaccggagccggcgccggggaccgc

SEQ ID NO: 40 - DNA, 1845 bp, Artificial Sequence
  Lasso cyclase
atggcgaagttgagtgctgggtttgtggtcctgccggacaccgccgacggtgcggagatccgggcgctcgtaccgttcgcgcctgcccgagtgatcgcgcacgcctccgggcggccgtggctggtcggcgagtggtctcagggtcaggtgagggtggcttcggcgggtccggtacgtctggccgtgagcggcacgtgtccggtcaccgacgacgggctggcgcggattgccgcccgtatctcccggctgtccgaggtggaccgggccgttcccgcgctgctggggtcctgccacatcgtggcatcggtggacgggcacgtccggttccagggcagcgcctccgggctgcgtcgcgtgttccacacccgggtgaacggggtgcgggtggccgcggaccggtccgatgtgctcgcggcgatgaccggtgccggggtggacgaggagacgctggcactgcacgtcgcatgcggtttgcaggtgccctttccggccaacgcgcgcagcgcgtggtccggcatcagcacgctcgcgccggagaactgtctgctgtgggagggcgaccgggaccgggaaacggtgtggtggcgtccgcccgagccggaccggtcgttgcgggaggggacggcagcggtacgggacaccctggctgccgtagtgggccgacaagcgcccgtcgaaggccggttgagtgcggatctgtccggcgggctggattcgacgtcgctgtgttttctggcggcccggcacacaccggagctgctgaccttccggtggggcgaggccgacgcgggcaatgacgacgcggtctacgcggggcaggcggcgcggctgctcgaccgtgccgagcacctcgtcgtgccgcagcacgaactccccgacatcttcgccgacccggccctcgcggtgtccgccgaggagccgctgtcgctgacgcgcgcgacggcccggatccgccggggcgcccgactgctcgcggaccgcggctcccgtcggcatctggccgggcacggcggcgacgagctgttctccccgatgccctcctatctgcatcagttgatgcgccgtcatccggtgacggcgctcaagcacgtacgggcgcactgcgcgctgaagcgctggccgctgaaggcgacgcttgccgcgctcgcgcagccgggaagtctccccgcgtggtggcgggagcaggccgggctgctgaccgagccccggccgccgctgcggcgcccggcgctgggctggggtctgggacccgtacgtgcggcgccctggatgacccccgagggcgtcgagctggcgcgggaggcgctgcgccggaccgccgagtgggcgcagccgctcgccgaggacctcgccacccacacgatggtgctcatgatccgttcgaacacggccagttaccggttgctggcgcggctgtacgccgaggccggtgtggagctggacatgccgtatctggacgacgccgtgatcgacgcggtgctccgggtaccggcgcacgcgcacgccggaccgtggcggtacaagcccctgctggccgatgcgatgcacggcatcgtgccggacggcatccgggcgcggtccaccaagggcgagttcggtgaggacatcagaagggggctgcgtcgcaacctgcccgccatcctggacctcttcgccgattccgagctggcggcccgcggtctgatcgacaccggcgcgctccggctgcggctgggcgggccgcagccggacaacaccaccgcccaggcgctggagagcctgctcggctgcgagacctggctgcgcaccacgtccggccccggcaccatcccccgtgccggcgacggcaccgttccctcggaggtc

SEQ ID NO: 41 - DNA, 252 bp, Artificial Sequence
  Lasso RRE
atgcggctccatcccgacgtctccatgacggacaccgacgacggcacggtgctgctgcatcagcgcaccggccgctactggcagctcaacgtcaccggcagccgcgtgctgcaccggctgctggacggggacacccccgagaccatcgccgacggcctcgccgccgcgcacggcatcgacccgcagcgggcccggcaggatgtcggcgccgtgatcgagcaactgcgcaccgccgagctgacggtggcctca

SEQ ID NO: 42 - protein, 15 aa, Artificial Sequence
  Core Peptide for Lasso 42
GNWHGTAPDWFFNYY

SEQ ID NO: 43 - protein, 17 aa, Artificial Sequence
  Core Peptide for Lasso 43
GNWHGTAPDWFFNYYWW

SEQ ID NO: 44 - protein, 16 aa, Artificial Sequence
  Core Peptide for Lasso 44
GNWHGTAPDWFFNYYA

SEQ ID NO: 45 - protein, 16 aa, Artificial Sequence
  Core Peptide for Lasso 45
GNWHGTAPDWFFNYYF

SEQ ID NO: 46 - protein, 16 aa, Artificial Sequence
  Core Peptide for Lasso 46
GNWHGTAPDWFFNYYY

SEQ ID NO: 47 - protein, 19 aa, Artificial Sequence
  Core Peptide for Lasso 47
GNWHGTAPDWFFNYYNYYW

SEQ ID NO: 48 - protein, 19 aa, Artificial Sequence
  Core Peptide for Lasso 48
GNWHGTAPDWFFNYYNIIW

SEQ ID NO: 49 - protein, 22 aa, Artificial Sequence
  Core Peptide for Lasso 49
GNWHGTAPDWFFNYYAHLDIIW

SEQ ID NO: 50 - protein, 16 aa, Artificial Sequence
  Core Peptide for Lasso 50
  misc_feature (16)..(16): Xaa = (2S)-2-amino-3-(1H-pyrrolo[5,4-b]pyridin- 3-yl)propanoic (Aza derivative of Tryptophan)
GNWHGTAPDWFFNYYX

SEQ ID NO: 51 - protein, 16 aa, Artificial Sequence
  Core Peptide for Lasso 51
  misc_feature (16)..(16): Xaa is Tryptophan having a C-terminal methyl ester group (-CO2Me) in place of the carboxylic acid group (-CO2H)
GNWHGTAPDWFFNYYX

SEQ ID NO: 52 - protein, 16 aa, Artificial Sequence
  Core Peptide for Lasso 52
  misc_feature (16)..(16): Xaa is Tryptophan having a C-terminal benzyl ester group (-CO2Bn) in place of the carboxylic acid group (-CO2H)
GNWHGTAPDWFFNYYX

SEQ ID NO: 53 - protein, 16 aa, Artificial Sequence
  Core Peptide for Lasso 53
  misc_feature (16)..(16): Tryptophan having a C-terminal amide group (-CONH2) in place of the carboxylic acid group (-CO2H)
GNWHGTAPDWFFNYYX

SEQ ID NO: 54 - protein, 16 aa, Artificial Sequence
  Core Peptide for Lasso 54
  misc_feature (16)..(16): Xaa is 2-naphthylalanine
GNWHGTAPDWFFNYYX

SEQ ID NO: 55 - protein, 19 aa, Artificial Sequence
  Core Peptide for Lasso 55
GNWHGTAPDWFFNYYWIIW

SEQ ID NO: 56 - protein, 19 aa, Artificial Sequence
  Core Peptide for Lasso 56
GNWHGTSPDWFFNYYWIIW

SEQ ID NO: 57 - protein, 43 aa, Artificial Sequence
  Precursor peptide for Lasso 42
MTEQSEQTPAEYIPPMLVEVGEFTEDTLGNWHGTAPDWFFNYY

SEQ ID NO: 58 - protein, 45 aa, Artificial Sequence
  Precursor peptide for Lasso 43
MTEQSEQTPAEYIPPMLVEVGEFTEDTLGNWHGTAPDWFFNYYWW

SEQ ID NO: 59 - protein, 44 aa, Artificial Sequence
  Precursor peptide for Lasso 44
MTEQSEQTPAEYIPPMLVEVGEFTEDTLGNWHGTAPDWFFNYYA

SEQ ID NO: 60 - protein, 44 aa, Artificial Sequence
  Precursor peptide for Lasso 45
MTEQSEQTPAEYIPPMLVEVGEFTEDTLGNWHGTAPDWFFNYYF

SEQ ID NO: 61 - protein, 44 aa, Artificial Sequence
  Precursor peptide for Lasso 46
MTEQSEQTPAEYIPPMLVEVGEFTEDTLGNWHGTAPDWFFNYYY

SEQ ID NO: 62 - protein, 47 aa, Artificial Sequence
  Precursor peptide for Lasso 47
MTEQSEQTPAEYIPPMLVEVGEFTEDTLGNWHGTAPDWFFNYYNYYW

SEQ ID NO: 63 - protein, 47 aa, Artificial Sequence
  Precursor peptide for Lasso 48
MTEQSEQTPAEYIPPMLVEVGEFTEDTLGNWHGTAPDWFFNYYNIIW

SEQ ID NO: 64 - protein, 50 aa, Artificial Sequence
  Precursor peptide for Lasso 49
MTEQSEQTPAEYIPPMLVEVGEFTEDTLGNWHGTAPDWFFNYYAHLDIIW

SEQ ID NO: 65 - protein, 44 aa, Artificial Sequence; identical to SEQ ID NO: 19
  Precursor peptide for Lasso 50
  misc_feature (44)..(44): Xaa = (2S)-2-amino-3-(1H-pyrrolo[5,4-b]pyridin- 3-yl)propanoic (Aza derivative of Tryptophan)

SEQ ID NO: 66 - protein, 44 aa, Artificial Sequence; identical to SEQ ID NO: 19
  Precursor peptide for Lasso 51
  misc_feature (44)..(44): Xaa is Tryptophan having a C-terminal methyl ester group (-CO2Me) in place of the carboxylic acid group (-CO2H)

SEQ ID NO: 67 - protein, 44 aa, Artificial Sequence; identical to SEQ ID NO: 19
  Precursor peptide for Lasso 52
  misc_feature (44)..(44): Xaa is Tryptophan having a C-terminal benzyl ester group (-CO2Bn) in place of the carboxylic acid group (-CO2H)

SEQ ID NO: 68 - protein, 44 aa, Artificial Sequence; identical to SEQ ID NO: 19
  Precursor peptide for Lasso 53
  misc_feature (44)..(44): Tryptophan having a C-terminal amide group (-CONH2) in place of the carboxylic acid group (-CO2H)

SEQ ID NO: 69 - protein, 44 aa, Artificial Sequence; identical to SEQ ID NO: 19
  Precursor peptide for Lasso 54
  misc_feature (44)..(44): Xaa is 2-naphthylalanine

SEQ ID NO: 70 - protein, 47 aa, Artificial Sequence
  Precursor peptide for Lasso 55
MTEQSEQTPAEYIPPMLVEVGEFTEDTLGNWHGTAPDWFFNYYWIIW

SEQ ID NO: 71 - protein, 47 aa, Artificial Sequence
  Precursor peptide for Lasso 56
MTEQSEQTPAEYIPPMLVEVGEFTEDTLGNWHGTSPDWFFNYYWIIW

SEQ ID NO: 72 - protein, 22 aa, Artificial Sequence
  synthetic peptide
SEKDREGVRIRERGAVRKSLPA

(19) United States
(12) Patent Application Publication
(10) Pub. No.: US 2023/0076411 A1
(43) Pub. Date: Mar. 9, 2023

(54) LASSO PEPTIDES FOR TREATMENT OF CANCER

(71) Applicant: Lassogen, Inc., San Diego, CA (US)

(72) Inventor: Mark J. Burk, San Diego, CA (US)

(21) Appl. No.: 17/790,103

(22) PCT Filed: Jan. 5, 2021
(86) PCT No.: PCT/US2021/012191

§ 371 Date: Jun. 29, 2022

Related U.S. Application Data
(60) Provisional application No. 62/957,762, filed on Jan. 6, 2020.
(60) Provisional application No. 62/980,918, filed on Feb. 24, 2020.

(51) Int. Cl. A61K 38/16; A61K 38/12; C07K 7/64; C07K 14/195; C07K 14/72; A61P 35/00

(52) U.S. Cl. CPC A61K 38/164; A61K 38/12; C07K 7/64; C07K 14/195; C07K 14/723; A61P 35/00

(57) ABSTRACT

Provided herein are endothelin receptor antagonistic lasso peptides and related compositions and methods for the management, prevention and/or treatment of an endothelin B receptor (ETBR)-mediated proliferative disease, such as cancer. Biosynthetic methods for producing the lasso peptides are also provided. In some embodiments, the method comprises administering to the subject a therapeutic effective amount of a lasso peptide, wherein the lasso peptide comprises an amino acid sequence selected from SEQ ID NOS:1-17 and 42-56. In particular embodiments, the lasso peptide is GI-D9 cyclized.

### CROSS-REFERENCE TO RELATED APPLICATION

This application claims the benefit of U.S. Ser. No. 62/957,762 filed Jan. 6, 2020 and U.S. Ser. No. 62/980,918 filed Feb. 24, 2020, the content of each of which is incorporated by reference in its entirety.

### 1. FIELD

The field of invention covers methods for the production and use of lasso peptides as a novel approach to treat cancer.

### 2. BACKGROUND

Cancer is a complex disease commonly caused by DNA damage, genetic mutations, or epigenetic modifications that support dysfunctional cellular signaling and aberrant cellular behavior leading to uncontrolled cellular growth. Over two hundred different forms of cancer are known and hundreds of drugs and drug combinations have been approved as treatments for specific cancer indications. Survival rates remain low and prevalence is increasing for many cancers (American Cancer Society. Cancer Facts & Figures 2019. Atlanta: American Cancer Society; 2019), and there exists a need for new therapeutic approaches with improved performance to combat these serious malignancies.

Endothelin receptors are transmembrane G protein-coupled receptors (GPCRs) normally expressed on the surface of endothelial cells lining the inner wall of blood and lymphatic vessels. Two main receptors, endothelin receptor type A (ETAR) and endothelin receptor type B (ETBR), regulate normal vascular function by binding to one of three cognate endothelin ligands, endothelin-1 (ET-1), endothelin-2 (ET-2), or endothelin-3 (ET-3). Endothelin-induced intracellular signaling transduced by activated ETAR and ETBR controls vascular homeostasis by balancing vasoconstriction, vasodilation, angiogenesis, lymphangiogenesis, cell proliferation, and cell survival (Vignon-Zellweger, N., et al., Endothelin and endothelin receptors in the renal and cardiovascular systems, Life Sciences, 2012, 91, 490-500). ETBR activation specifically mediates the release of relaxing factors such as nitric oxide, prostacyclin, and endothelium-derived hyperpolarizing factor, increases in [Ca²⁺]ᵢ, protein kinase C, mitogen-activated protein kinase, and other pathways involved in vascular contraction and cell growth (Mazzuca, M. Q., Khalil, R. A. Vascular endothelin receptor type B: structure, function and dysregulation in vascular disease, Biochem Pharmacol. 2012; 84(2): 147-162).

Endothelin receptor antagonists have been reported in the literature and have been largely studied in the context of pulmonary arterial hypertension (PAH) and other cardiovascular diseases (Aubert, J., et al., Endothelin receptor antagonists beyond pulmonary arterial hypertension, cancer and fibrosis, J. Med. Chem. 2016, 59, 8168-8188; Davenport, A. P., et al., New drugs and emerging targets in endothelin signaling pathway and prospects for precision medicine, Physiol. Res., 2018, 67 (Suppl. 1), S37-S54). For example, a modified tetrapeptide molecule BQ-788 was initially developed to characterize the physiological and pathological roles of endothelin receptors in the context of hypertension and pulmonary diseases (Ishikawa, K. et al., Biochemical and pharmacological profile of a potent and selective endothelin B-receptor antagonist, BQ-788, Proc. Nat. Acad. Sci. USA, 1994, 91, 4892-4896). Most endothelin receptor antagonists are either ETAR selective or antagonize both ETAR and ETBR, and several have been approved for treating PAH and related disorders. Relatively few potent and selective ETBR antagonists have been described (Aubert, J., et al., Endothelin receptor antagonists beyond pulmonary arterial hypertension, cancer and fibrosis, J. Med. Chem. 2016, 59, 8168-8188), and no selective ETBR antagonist has been approved for commercial use. Importantly, there is also no cancer medication approved or under clinical studies, which medication advances its anti-cancer therapeutic effect through ETBR antagonism.

Despite tremendous efforts in the field of cancer medication, cancer mortality rate remains high across the globe. Thus, there remains urgent needs for the development of effective new cancer medication. The present disclosure meets this need.

### 3. SUMMARY

Provided herein are endothelin receptor antagonistic lasso peptides and related compositions and methods for the management, prevention and/or treatment of an endothelin B receptor (ETBR)-mediated proliferative disease, such as cancer. Biosynthetic methods for producing the lasso peptides are also provided.

Particularly, in one aspect of the present disclosure, provided herein are a method of managing, preventing, or treating an endothelin B receptor (ETBR)-mediated proliferative disease producing neoplastic cells in a subject. In some embodiments, the method comprises administering to the subject a therapeutic effective amount of a lasso peptide, wherein the lasso peptide comprises an amino acid sequence selected from SEQ ID NOS:1-17 and 42-56. In particular embodiments, the lasso peptide is G1-D9 cyclized.

In some embodiments, upon administration, the lasso peptide competes with endothelin for the binding with ETBR. Particularly, in some embodiments, the endothelin is endothelin 1, endothelin 2 and/or endothelin 3. In some embodiments, upon administration, the lasso peptide preferentially binds to ETBR over endothelin A receptor (ETAR). In some embodiments, upon administration, the lasso peptide specifically inhibits ETBR. In some embodiments, upon administration, the lasso peptide preferentially binds to ETBR1 over ETBR2. In some embodiments, upon administration, the lasso peptide specifically inhibits ETBR1.

In some embodiments, upon administration, the lasso peptide (a) inhibits ETBR-mediated signaling pathway; and/or (b) downregulates ETBR expression on the surface of the neoplastic cells and/or endothelial cells in the microenvironment of the neoplastic cells. In particular embodiments, the inhibition of the ETBR-mediated signaling pathway is measured by (a) inhibition of release of relaxing factors; (b) upregulation of intercellular adhesion molecule-1 (ICAM-1) expression and clustering; (c) increase in migration of intraepithelial tumor infiltrating leukocytes (TILs) into the microenvironment of the neoplastic cells; (d) inhibition of angiogenesis in the microenvironment of neoplastic cells; (e) inhibition of growth and/or metastasis of neoplastic cells; and/or (f) increase in apoptosis of neoplastic cells.

In some embodiments, upon administration, the lasso peptide inhibits release of relaxing factors selected from nitric oxide, prostacyclin, and endothelium-derived hyperpolarizing factor, Ca²⁺, protein kinase C, mitogen-activated protein kinase, or any combination thereof.

In some embodiments, upon administration, the lasso peptide increases migration of TILs into the microenvironment of the neoplastic cells, and the TILs comprise neutrophils, T cells, B cells, NK cells, monocytes or a combination thereof. In particular embodiments, the TILs comprise macrophages and/or dendritic cells.

In some embodiments, upon administration of the lasso peptide, the maximal percent inhibition of the ETBR-mediated signaling pathway is at least about 10%, 20%, 30%, 40%, 45%, 50%, 55%, 60%, 65%, 70%, 75%, 80%, 85%, 90%, 95%, or 100%. In some embodiments, upon administration of the lasso peptide, the maximal percent downregulation of ETBR expression is at least about 10%, 20%, 30%, 40%, 45%, 50%, 55%, 60%, 65%, 70%, 75%, 80%, 85%, 90%, 95%, or 100%.

In some embodiments, the proliferative disease being treated produces neoplastic cells expressing ETBR. In some embodiments, the subject being treated expresses ETBR in endothelial cells of vasculature in the microenvironment of the neoplastic cells. In particular embodiments, the ETBR being expressed is ETBR1, ETBR2 or both ETBR1 and ETBR2.

In some embodiments, the proliferative disease being treated is cancer. In particular embodiments, the cancer is selected from breast cancer, pancreatic cancer (e.g., pancreatic adenocarcinoma), hepatocellular carcinoma, prostate cancer, ovarian cancer, gastric cancer, glioblastoma, melanoma, cancer of the head and neck, colorectal cancer, bladder cancer, vulvar cancer, esophageal squamous cell carcinoma, renal cancer (e.g., clear-cell renal cell carcinoma), cervical cancer, salivary gland carcinoma, lung cancer (e.g., non-small cell lung cancer and small-cell lung cancer), multiple myeloma, or Kaposi's sarcoma. In particular embodiments, the proliferative disease being treated is melanoma. In particular embodiments, the proliferative disease being treated is ovarian cancer.

In some embodiments, the lasso peptide is conjugated to an agent. In particular embodiments, the lasso peptide is conjugated or fused to an agent selected from the group consisting of a radioisotope, a metal chelator, an enzyme, a protein, a peptide, an antibody, an antibody fragment, a nanobody, a fluorescent compound, a bioluminescent compound, and a chemiluminescent compound.

In some embodiments, the method further comprises co-administering to the subject a second therapeutic agent with the lasso peptide. In some embodiments, the second therapeutic agent is not a lasso peptide. In some embodiments, the second therapeutic agent is conjugated with the lasso peptide. In some embodiments, the second therapeutic agent is an immunotherapy or chemotherapy. In particular embodiments, the immunotherapy is an anti-cancer vaccine or an immune checkpoint modulator.

In another aspect of the present disclosure, a method of cell-free biosynthesis of a lasso peptide is provided. In some embodiments, the method comprises contacting a peptide comprising a sequence selected from SEQ ID NOS:1-34 and 42-71 with a lasso peptide biosynthesis component in a cell-free biosynthesis reaction mixture; and incubating the cell-free biosynthesis reaction mixture under a condition suitable for lasso formation to produce the lasso peptide. In alternative embodiments, provided herein is a method of cell-free biosynthesis of a lasso peptide having an amino acid sequence selected from SEQ ID NOS:1-17 and 42-56, where the method comprises contacting a peptide comprising a leader sequence and a lasso core peptide sequence that is selected from SEQ ID NOS:1-17 and 42-56 with a lasso peptide biosynthesis component in a cell-free biosynthesis reaction mixture; and incubating the cell-free biosynthesis reaction mixture under a condition suitable for lasso formation to produce the lasso peptide; where the lasso peptide biosynthesis component comprises a lasso peptidase capable of catalyzing removal of the leader sequence.

In particular embodiments of the cell-free biosynthesis method, the contacting step comprises adding a first nucleic acid sequence encoding the peptide into the cell-free biosynthesis reaction mixture, and wherein the cell-free biosynthesis reaction mixture comprises in vitro TX-TL machinery configured to express the peptide.

In particular embodiments of the cell-free biosynthesis method, the contacting step comprises adding a second nucleic acid sequence encoding the lasso peptide biosynthesis component to the cell-free biosynthesis reaction mixture, and wherein the cell-free biosynthesis reaction mixture comprises in vitro TX-TL machinery configured to express the lasso peptide biosynthesis component.

In some embodiments, the lasso peptide biosynthesis component comprises a lasso cyclase, and the contacting step comprises adding the second nucleic acid sequence encoding the lasso cyclase. In some embodiments, the lasso peptide biosynthesis component comprises a lasso peptidase and a lasso cyclase, and the contacting comprises adding the second nucleic acid sequence encoding the lasso cyclase and a third nucleic acid sequence encoding the lasso peptidase. In some embodiments, the lasso peptide biosynthesis component comprises a lasso cyclase and a post-translationally modified peptide (RiPP) recognition element (RRE), and the contacting comprises adding the second nucleic acid sequence encoding the lasso cyclase and a fourth nucleic acid sequence encoding the RRE. In some embodiments, the lasso peptide biosynthesis component comprises a lasso peptidase, a lasso cyclase and a post-translationally modified peptide (RiPP) recognition element (RRE), and the contacting comprises adding the second nucleic acid sequence encoding the lasso cyclase, a third nucleic acid sequence encoding the lasso peptidase and a fourth nucleic acid sequence encoding the RRE.

In various embodiments of the cell-free biosynthesis method described herein, at least two of the first, second, third and fourth nucleic acid sequences are in a same nucleic acid molecule. In some embodiments of the cell free-biosynthesis method described herein, the cell-free biosynthesis reaction mixture comprises cell extract or supplemented cell extract.

In another aspect of the present disclosure, provided herein is a method for producing a lasso peptide using a non-naturally occurring microbial organism. In some embodiments, the method comprises introducing into the microbial organism a first nucleic acid sequence encoding a peptide comprising a sequence of SEQ ID NOS:1-34 and 42-71 and a second nucleic acid sequence encoding a lasso peptide biosynthesis component; and culturing the microbial organism under a condition suitable for lasso formation to produce the lasso peptide. In alternative embodiments, provided herein is a method for producing a lasso peptide having an amino acid sequence selected from SEQ ID NOS:1-17 and 42-56, where the method comprises introducing into the microbial organism a first nucleic acid sequence encoding a peptide comprising a leader sequence and a lasso core peptide sequence that is selected from SEQ ID NOS:1-17 and 42-56 and a second nucleic acid sequence encoding a lasso peptide biosynthesis component; and culturing the microbial organism under a condition suitable for lasso formation to produce the lasso peptide; where the lasso peptide biosynthesis component comprises a lasso peptidase capable of catalyzing removal of the leader sequence.

In some embodiments of the method for producing a lasso peptide using a non-naturally occurring microbial organism, the lasso peptide biosynthesis component comprises a lasso cyclase, and the method comprises introducing into the microbial organism the second nucleic acid sequence encoding the lasso cyclase.

In some embodiments of the method for producing a lasso peptide using a non-naturally occurring microbial organism, the lasso peptide biosynthesis component comprises a lasso peptidase and a lasso cyclase, and the method comprises introducing the second nucleic acid sequence encoding the lasso cyclase and a third nucleic acid sequence encoding the lasso peptidase.

In some embodiments of the method for producing a lasso peptide using a non-naturally occurring microbial organism, the lasso peptide biosynthesis component comprises a lasso cyclase and a post-translationally modified peptide (RiPP) recognition element (RRE), and the method comprises introducing the second nucleic acid sequence encoding the lasso cyclase and a fourth nucleic acid sequence encoding the RRE.

In some embodiments of the method for producing a lasso peptide using a non-naturally occurring microbial organism, the lasso peptide biosynthesis component comprises a lasso peptidase, a lasso cyclase and a post-translationally modified peptide (RiPP) recognition element (RRE), and the method comprises introducing the second nucleic acid sequence encoding the lasso cyclase, a third nucleic acid sequence encoding the lasso peptidase, and a fourth nucleic acid sequence encoding the RRE.

In various embodiments of the method for producing a lasso peptide using a non-naturally occurring microbial organism, at least two of the first, second, third and fourth nucleic acid sequences are in a same nucleic acid molecule. In various embodiments, the microbial organism is E. coli, Vibrio natriegens, Burholderia spp., Corynebacterium glutamicum, or Sphingomaons subterranean. In various embodiments, the culturing is performed under a substantially anaerobic condition.

In particular embodiments of present method for producing the lasso peptide either via the cell-free method or the use of a non-naturally occurring microbial organism, the method comprises providing a peptide comprising an amino acid sequence selected from SEQ ID NOS:1-17 and 42-56, and contacting the provided peptide with the lasso peptide biosynthesis component comprising a lasso cyclase under a condition suitable for lasso formation to produce the lasso peptide. In specific embodiments, the lasso peptide biosynthesis component further comprises a lasso peptidase and/or a RRE.

In particular embodiments of present method for producing the lasso peptide either via the cell-free method or the use of a non-naturally occurring microbial organism, the method comprises providing a peptide comprising an amino acid sequence selected from SEQ ID NOS:18-34 and 57-71, and contacting the provided peptide with the lasso peptide biosynthesis component comprising a lasso peptidase and a lasso cyclase under a condition suitable for lasso formation to produce the lasso peptide. In specific embodiments, the lasso peptide biosynthesis component further comprises a lasso peptidase and/or a RRE.

In various embodiments of present method for producing the lasso peptide either via the cell-free method or the use of a non-naturally occurring microbial organism, the lasso cyclase comprises the sequence of SEQ ID NO:36. In various embodiments of present method for producing the lasso peptide either via the cell-free method or the use of a non-naturally occurring microbial organism, the lasso peptidase comprises the sequence of SEQ ID NO:35. In various embodiments of present method for producing the lasso peptide either via the cell-free method or the use of a non-naturally occurring microbial organism, the RRE comprises the sequence of SEQ ID NO:37.

In various embodiments of present method for producing the lasso peptide either via the cell-free method or the use of a non-naturally occurring microbial organism, the produced lasso peptide comprises an amino acid sequence selected from SEQ ID NOS:1-17 and 42-56, and the lasso peptide is G1-D9 cyclized.

In various embodiments of present method for producing the lasso peptide either via the cell-free method or the use of a non-naturally occurring microbial organism, the method further comprises isolating the lasso peptide from the cell-free biosynthesis reaction mixture of the culture medium of the microbial organism.

In related aspects of the present disclosure, provided herein are also biosynthesized lasso peptides produced using the present methods, and related compositions, including pharmaceutical compositions comprising the biosynthesized lasso peptides. In some embodiments, a pharmaceutical composition according to the present disclosure comprises the biosynthesized lasso peptide and a pharmaceutically acceptable carrier. In some embodiments, the pharmaceutical composition of the present disclosure further comprises a second therapeutic agent for managing, preventing or treating cancer. In specific embodiments, the second therapeutic agent is chemotherapy or immunotherapy for cancer. In specific embodiments, the second therapeutic agent is an anti-cancer vaccine or immune checkpoint modulator.

In related aspects of the present disclosure, provided herein are also a method for managing, preventing or treating a endothelin-B medicated proliferative disease in a subject using the biosynthesized lasso peptide and/or pharmaceutical composition as disclosed herein. In some embodiments, the method comprises administering a therapeutically effective amount of the biosynthesized lasso peptide or pharmaceutical composition comprising the biosynthesized lasso peptide.

### 4. BRIEF DESCRIPTION OF THE FIGURES

The details of one or more embodiments of the invention are set forth in the accompanying drawings and the description below. Other features, objects, and benefits of the invention will be apparent from the description and drawings, and from the claims. All publications, patents and patent applications cited herein are hereby expressly incorporated by reference for all purposes.

The embodiments of the description described herein are not intended to be exhaustive or to limit the disclosure to the precise forms disclosed in the following drawings or detailed description. Rather, the embodiments are chosen and described so that others skilled in the art can appreciate and understand the principles and practices of the description.

FIG. 1A is a schematic illustration of the of a lasso peptide with the characteristic lasso (lariat) topology.

FIG. 1B is a schematic illustration of the lasso peptide biosynthesis pathway, including the genes and gene products involved in the enzymatic reactions resulting in formation of matured lasso peptide having the characteristic lasso (lariat) topology.

FIG. 2 is a schematic illustration of the amino acid sequences of four lasso peptides according to specific embodiments of the present disclosure.

FIG. 3A shows the chemical structure of Compound BQ-788.

FIG. 3B shows a table summarizing IC₅₀ of BQ-788 against endothelin A receptor (ETAR) and endothelin B receptor (ETBR). As shown, BQ-788 selectively binds to ETBR over ETAR.

FIG. 4A shows effect of LAS-103 on inhibiting binding of radiolabeled endothelin-1 (ET-1) (¹²⁵I-ET-1) to endothelin B receptor (ETBR) expressed on the surface of CHO cells. FIG. 4B shows effect of LAS-103 on inhibiting binding of ¹²⁵I-ET-1 to endothelin A receptor (ETAR) expressed on the surface of CHO cells. As shown, LAS-103 selectively inhibits binding of ET1 to ETBR over ETAR.

FIG. 5 illustrates functions of endothelin B receptor (ETBR) in promoting cancer development.

FIG. 6 illustrates functions of endothelin B receptor (ETBR) antagonists in inhibiting cancer development.

FIG. 7 illustrates differential interactions of BQ-788 and LAS-101 with two subtypes of endothelin B receptor (ETBR), namely ETBR 1 predominantly expressed on endothelium cells and ETBR2 predominantly expressed on smooth muscle cells. As shown, unlike BQ-788, LAS-101 selectively inhibits ETBR1 over ETBR2.

FIG. 8 is a schematic illustration of certain cellular pathways mediated by endothelin B receptor (ETBR) expressed on endothelial cells. As shown, endothelin B receptor (ETBR) on endothelial cells blocks ICAM-1 expression and clustering, thereby blocking leukocytes in the blood vessel lumen from entering the tumor microenvironment.

FIG. 9 is a schematic illustration of cell-based biosynthesis process for production of lasso peptides according to certain embodiments of the present disclosure.

FIG. 10 is a schematic illustration of cell-free biosynthesis process for production of lasso peptides according to certain embodiments of the present disclosure.

FIG. 11 is a schematic illustration comparing cell-free and cell-based biosynthesis process for production of lasso peptides according to certain embodiments of the present disclosure.

FIG. 12 is a schematic illustration showing the process for evolving lasso peptides with successive accumulation of amino acid changes to properties of the optimize lasso peptide. Amino acids are represented as round balls in the lasso structure.

FIG. 13 is a schematic illustration showing biochemical conversion of lasso precursor peptides treated with biosynthetic proteins, such as a lasso peptidase (B) and a lasso cyclase (C).

### 5. DETAILED DESCRIPTION

The novel features of this invention are set forth specifically in the appended claims. A better understanding of the features and benefits of the present invention will be obtained by reference to the following detailed description that sets forth illustrative embodiments, in which the principles of the invention are utilized. To facilitate a full understanding of the disclosure set forth herein, a number of terms are defined below.

### 5.1 General Techniques

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (4th ed. 2012); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2nd ed. 2010). Molecular Biology of the Cell (6th Ed., 2014). Organic Chemistry, (Thomas Sorrell, 1999). March's Advanced Organic Chemistry (6^{th} ed. 2007). Lasso Peptides, (Li, Y.; Zirah, S.; Rebuffet, S., Springer; New York, 2015). Natural Products in Medicinal Chemistry, Methods and Principles in Medicinal Chemistry (Hanessian, S., ed., Wiley-VCH; 1st edition, 2014. Basic Principles of Drug Discovery and Development (Blass, B. Academic Press; 1st edition, 2015).

### 5.2 Terminology

Unless described otherwise, all technical and scientific terms used herein have the same meaning as is commonly understood by one of ordinary skill in the art. For purposes of interpreting this specification, the following description of terms will apply and whenever appropriate, terms used in the singular will also include the plural and vice versa. All patents, applications, published applications, and other publications are incorporated by reference in their entirety. In the event that any description of terms set forth conflicts with any document incorporated herein by reference, the description of term set forth below shall control.

The singular terms “a,” “an,” and “the” as used herein include the plural reference unless the context clearly indicates otherwise.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The term “substantially” means that something takes place, as a function or activity, to provide the expected outcome or result to a large degree and to a great extent, but still not to the fullest extent. For example, if a lasso peptide is substantially purified, the lasso peptide is isolated and purification steps afford the lasso peptide at purity level above 90% and as high as 99.99%.

The term “substantially all” refers to at least about 60%, at least about 65%, at least about 70%, at least about 75%, at least about 80%, at least about 85%, at least about 90%, at least about 95%, at least about 98%, at least about 99%, or about 100%.

The terms “oligonucleotide” and “nucleic acid” refer to oligomers of deoxyribonucleotides (e.g., DNA) or ribonucleotides (e.g., RNA) and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer, M. A., et al., Nucleic Acid Res., 1991, 19, 5081-1585; Ohtsuka, E. et al., J. Biol. Chem., 1985, 260, 2605-2608; and Rossolini, G. M., et al., Mol. Cell. Probes, 1994, 8, 91-98). “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a lasso peptide of the present disclosure may include a bacterial and archaea host cells into which nucleic acids encoding the lasso peptide component have been introduced. Suitable host cells are disclosed below.

Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

The term “amino acid” refers to naturally occurring and non-naturally occurring alpha-amino acids, as well as alpha-amino acid analogs and amino acid mimetics that function in a manner similar to the naturally occurring alpha-amino acids. Naturally encoded amino acids are the 22 common amino acids (alanine, arginine, asparagine, aspartic acid, cysteine, glutamine, glutamic acid. glycine, histidine, isoleucine, leucine, lysine, methionine, phenylalanine, proline, serine, threonine, tryptophan, tyrosine, valine, pyrrolysine and selenocysteine). Amino acid analogs or derivatives refers to compounds that have the same basic chemical structure as a naturally occurring amino acid, i.e., a carbon that is bound to a hydrogen, a carboxyl group, an amino group, and a side chain R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms “non-natural amino acid” or “non-proteinogenic amino acid” or “unnatural amino acid” or “non-canonical” refer to alpha-amino acids that contain different side chains (different R groups) relative to those that appear in the twenty-two common or naturally occurring amino acids listed above. In addition, these terms also can refer to amino acids that are described as having D-stereochemistry, rather than L-stereochemistry of natural amino acids, despite the fact that some amino acids do occur in the D-stereochemical form in nature (e.g., D-alanine and D-serine).

The term “peptide” as used herein refers to a polymer chain containing between two and fifty (2-50) amino acid residues. The terms apply to naturally occurring amino acid polymers as well as amino acid polymers in which one or more amino acid residues is a non-naturally occurring amino acid, e.g., an amino acid analog or non-natural amino acid.

The terms “polypeptide” and “protein” are used interchangeably herein to refer to a polymer of greater than about fifty (50) amino acid residues. That is, a description directed to a polypeptide applies equally to a description of a protein, and vice versa. The terms apply to naturally occurring amino acid polymers as well as amino acid polymers in which one or more amino acid residues is a non-naturally occurring amino acid, e.g., an amino acid analog. As used herein, the terms encompass amino acid chains of any length, including full length proteins (i.e., antigens), wherein the amino acid residues are linked by covalent peptide bonds.

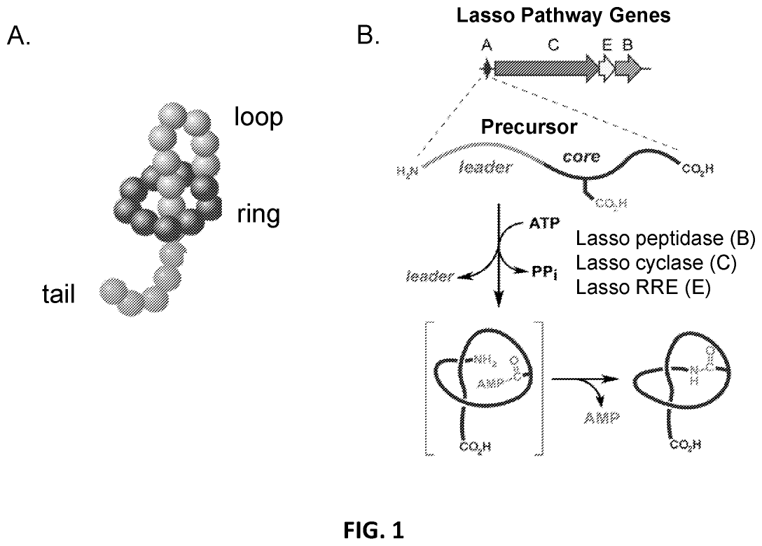

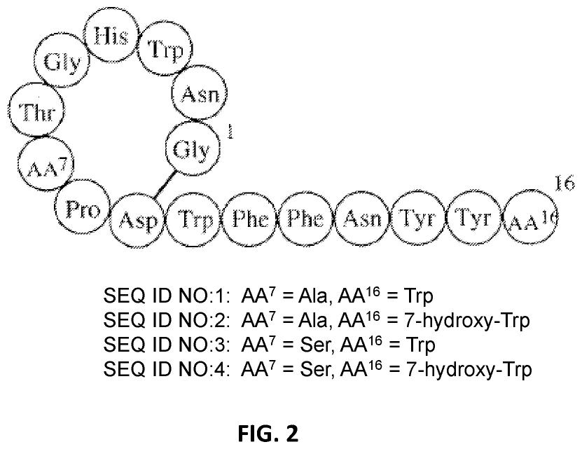

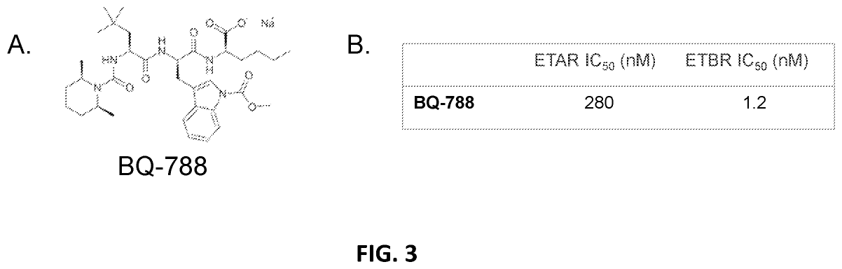

The terms “lasso peptide” and “lasso” are used interchangeably herein, and is used to refer to a class of peptide or polypeptide having the general lariat-like topology as exemplified in FIG. 1A. As shown in the figure, the lariat-like topology can be generally divided into a ring portion, a loop portion, and a tail portion. Particularly, a region on one end of the peptide forms the ring around the tail on the other end of the peptide, the tail is threaded through the ring, and a middle loop portion connects the ring and the tail, together forming the lariat-like topology. Particularly, the amino acid residues that are joined together to form the ring are herein referred to as the “ring-forming amino acid.” A ring-forming amino acid can located at the N- or C-terminus of the lasso peptide (“terminal ring-forming amino acid”), or in the middle (but not necessarily the center) of a lasso peptide (“internal ring-forming amino acid”). For example, the term “G1-D9 cyclized” as used herein when referring to a lasso peptide, means that the lasso peptide has a N-terminal ring-forming amino acid of a glycine residue (G1) and an internal ring-forming amino acid of an aspartate residue at position 9 (D9), where the amino group of G1 and the carboxyl group of D9 form an isopeptide bond, thus forming the ring portion of the lasso peptide. The fragment of a lasso peptide between and including the two ring-forming amino acid residues is the ring portion; the fragment of a lasso peptide between the internal ring-forming amino acid and where the peptide threaded through the plane of the ring is the loop portion; and the remaining fragment of a lasso peptide starting from where the peptide is threaded through the plane of the ring is the tail portion. In addition to the lariat-like topology, additional topological features of a lasso peptide may further include intra-peptide disulfide bonding, such as disulfide bond(s) between the tail and the ring, between the ring and the loop, and/or between different locations within the tail. As used herein, “lasso peptide” or “lasso” refers to both naturally-existing peptides and artificially produced peptides that have the lariat-like topology as described herein. Similarly, “lasso peptide” or “lasso” also refers to non-naturally occurring analogs, derivatives, or variants of a naturally occurring lasso peptide, which analogs, derivatives or variants are also lasso peptides themselves.

The term “lasso precursor peptide” or “precursor peptide” as used herein refers to a precursor that is processed into or otherwise forms a lasso peptide. In some embodiments, a lasso precursor peptide comprises at least one a lasso core peptide portion. In some embodiments, a lasso precursor peptide comprises one or more amino acid residues or amino acid fragments that do not belong to a lasso core peptide, such as a leader sequence that facilitates recognition of the lasso precursor peptide by one or more lasso processing enzymes. In some embodiments, the lasso precursor peptide is enzymatically processed into a lasso peptide by removing the amino acid residues or fragments that do not belong to a lasso core peptide. In some embodiments, a lasso precursor peptide is the substrate of an enzyme that cleaves off the additional amino acid residues or fragments from a lasso precursor peptide to produce the lasso peptide. As used herein, the enzyme capable of catalyzing this reaction is referred to as the “lasso peptidase.”

The term “lasso core peptide” or “core peptide” refers to the peptide or the peptide segment of the precursor peptide that is processed into or otherwise forms a lasso peptide having the lariat-like topology. As used herein, the enzyme capable of catalyzing cyclization the ring portion of a lasso core peptide is referred to as the “lasso cyclase.” As used herein, a core peptide may have the same amino acid sequence as a lasso peptide, but has not matured to have the lariat-like topology of a lasso peptide. In various embodiments, core peptides can have different lengths of amino acid sequences. In some embodiments, the core peptide is at least about 5 amino acid long. In some embodiments, the core peptide is at least about 10 amino acid long. In some embodiments, the core peptide is at least about 11 amino acid long. In some embodiments, the core peptide is at least about 12 amino acid long. In some embodiments, the core peptide is at least about 13 amino acid long. In some embodiments, the core peptide is at least about 14 amino acid long. In some embodiments, the core peptide is at least about 15 amino acid long. In some embodiments, the core peptide is at least about 16 amino acid long. In some embodiments, the core peptide is at least about 17 amino acid long. In some embodiments, the core peptide is at least about 18 amino acid long. In some embodiments, the core peptide is at least about 19 amino acid long. In some embodiments, the core peptide is at least about 20 amino acid long. In some embodiments, the core peptide is at least about 25 amino acid long. In some embodiments, the core peptide is at least about 30 amino acid long. In some embodiments, the core peptide is at least about 35 amino acid long. In some embodiments, the core peptide is at least about 40 amino acid long. In some embodiments, the core peptide is at least about 45 amino acid long. In some embodiments, the core peptide is at least about 50 amino acid long. In some embodiments, the core peptide is at least about 55 amino acid long. In some embodiments, the core peptide is at least about 60 amino acid long. In some embodiments, the core peptide is at least about 65 amino acid long.

The term “biosynthetic gene cluster” as used herein refers to one or more nucleic acid molecule(s) independently or jointly comprising one or more coding sequences for a precursor and processing machinery capable of maturing the precursor into a biosynthetic end product. The coding sequences can comprise multiple open reading frames (ORFs) each independently coding for one component of the precursor and processing machinery. Alternatively, the coding sequences can comprise an ORF coding for two or more components of the precursor and processing machinery fused together, as further described herein. A biosynthetic gene cluster can be identified and isolated from the genome of an organism. Computer-based analytical tools can be used to mine genomic information and identify biosynthetic gene clusters encoding lasso peptides. For example, the genome-mining tool known as Rapid ORF Description and Evaluation Online (RODEO) has been used to identify more than a thousand of lasso biosynthetic gene clusters based on available genomic information (Tietz et al. Nat Chem Biol. 2017 May; 13(5): 470-478). Alternatively, a biosynthetic gene cluster can be assembled by artificially producing and combining the nucleic acid components of the gene cluster, using genetic manipulating methods and technology known in the art.

Some naturally existing lasso peptides are encoded by a lasso peptide biosynthetic gene cluster, which typically comprises three main genes: one encodes for a lasso precursor peptide (referred to as Gene A), and two encode for processing enzymes including a lasso peptidase (referred to as Gene B) and a lasso cyclase (referred to as Gene C). The lasso precursor peptide comprises a lasso core peptide and additional peptidic fragments known as the “leader sequence” that facilitates recognition and processing by the processing enzymes. The leader sequence may determine substrate specificity of the processing enzymes. The processing enzymes encoded by the lasso peptide gene cluster convert the lasso precursor peptide into a matured lasso peptide having the lariat-like topology. Particularly, the lasso peptidase removes from the precursor peptide the additional portion that is not the lasso core peptide, and the lasso cyclase cyclize a terminal portion of the core peptide around a terminal tail portion to form the lariat-like topology.

Some lasso gene clusters further encodes for additional protein elements that facilitates the post-translational modification, including a facilitator protein known as the post-translationally modified peptide (RiPP) recognition element (RRE). A lasso peptide biosynthetic gene clusters may encode two or more of lasso peptidase, lasso cyclase and RRE as different domains in the same protein. Some lasso gene clusters further encodes for lasso peptide transporters, kinases, or proteins that play a role in immunity, such as isopeptidase. (Burkhart, B. J., et al., Nat. Chem. Biol., 2015, 11, 564-570; Knappe, T. A. et al., J. Am. Chem. Soc., 2008, 130, 11446-11454; Solbiati, J. O. et al. J. Bacteriol., 1999, 181, 2659-2662; Fage, C. D., et al., Angew. Chem. Int. Ed., 2016, 55, 12717-12721; Zhu, S., et al., J. Biol. Chem. 2016, 291, 13662-13678).

The term “lasso peptide biosynthesis component” as used herein refer to a protein comprising one or more of (i) a lasso peptidase, (ii) a lasso cyclase, and (iii) RRE.

The terms “cell-free biosynthesis” and “CFB” are used interchangeably herein and refer to an in vitro (outside the cell) biosynthetic process for the production of one or more peptides or proteins. In some embodiments, cell-free biosynthesis occurs in a “cell-free biosynthesis reaction mixture” or “CFB reaction mixture” which provides various components, such as RNA, proteins, enzymes, co-factors, natural products, small molecules, organic molecules, to carry out protein synthesis outside a living cell. In some embodiments, the CFB reaction mixture can comprise one or more cell extracts or supplemented cell extracts, or commercially available cell-free reaction media (e.g. PURExpress®). Exemplary CFB methods and systems, including those involving the use of in vitro TX-TL, are described in Culler, S. et al., PCT Application WO2017/031399 A1, and is incorporated herein by reference.

As used herein, the terms “in vitro transcription and translation” and “in vitro TX-TL” are used interchangeably and refer to a biosynthetic process outside an intact cell, where genes or oligonucleotides are transcribed into messenger ribonucleic acids (mRNAs), and mRNAs are translated into proteins or peptides. As used herein, the term “in vitro TX-TL machinery” refers to the components that act in concert to carry out the in vitro TX-TL. For the sole purpose of illustration, and by way of non-exhaustive and non-limiting examples, in some embodiments, an in vitro TX-TL machinery comprises enzyme(s) and co-factor(s) that carry out DNA transcription and/or mRNA translation. In some embodiments, an in vitro TX-TL machinery further comprises other small organic or inorganic molecules, such as amino acids, tRNAs or ATP, that facilitate the DNA transcription and/or mRNA translation. Various cellular components known to participate in in vivo transcription and translation can form part of the in vitro TX-TL machinery, see for example, Matsubayashi et al, “Purified cell-free systems as standard parts for synthetic biology.” Curr Opin Chem Biol. 2014 October; 22:158-62; Li, et al. “Improved cell-free RNA and protein synthesis system.” PLoS One. 2014 Sep. 2; 9 (9):e106232. In some embodiments, different components can be provided individually and combined to assemble the in vitro TX-TL machinery. Exemplary ways of providing the in vitro TX-TL machinery components include recombinantly production, synthesis, and isolation from a cell. In some embodiments, the in vitro TX-TL machinery is provided in the form of one or more cell extract, or one or more supplemented cell extract that comprises the in vitro TX-TL machinery.

The term “condition suitable for lasso formation,” depending on the context, may refer to, for example, a condition suitable for the expression of one or more protein products in a bacterial host (e.g., a lasso precursor peptide, or a processing enzyme). Exemplary suitable conditions included are not limited to a suitable culturing condition of the bacterial host that enable the protein synthesis and transportation in the host cell. Additionally or alternatively, depending on the context, the term “condition suitable for lasso formation” may refer to, for example, a condition suitable for post-translational modification of a lasso precursor peptide. Exemplary suitable conditions include but are not limited to a suitable temperature and/or incubation time for a lasso cyclase and/or lasso peptidase to process the lasso precursor in to a matured lasso peptide.

The terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

The term “naturally occurring” or “natural” or “native” when used in connection with naturally occurring biological materials such as nucleic acid molecules, oligonucleotides, amino acids, polypeptides, peptides, metabolites, small molecule natural products, host cells, and the like, refers to materials that are found in or isolated directly from Nature and are not changed or manipulated by humans. The term “natural” or “naturally occurring” refers to organisms, cells, genes, biosynthetic gene clusters, enzymes, proteins, oligonucleotides, and the like that are found in Nature and are unchanged relative to these components found in Nature. The term “wild-type” refers to organisms, cells, genes, biosynthetic gene clusters, enzymes, proteins, oligonucleotides, and the like that are found in Nature and are unchanged relative to these components found in Nature (in the wild).

The term “non-naturally occurring” or “non-natural” or “unnatural” or “non-native” or “non-canonical” as used herein refer to a material, substance, molecule, cell, enzyme, protein, peptide, or amino acid that is not known to exist or is not found in Nature or that has been structurally modified and/or synthesized by humans. The term “non-natural” or “unnatural” or “non-naturally occurring” when used in reference to a microbial organism or microorganism or cell extract or gene or biosynthetic gene cluster of the invention is intended to mean that the microbial organism or derived cell extract or gene or biosynthetic gene cluster has at least one genetic alteration not normally found in a naturally occurring strain or a naturally occurring gene or biosynthetic gene cluster of the referenced species, including wild-type strains of the referenced species. Genetic alterations include, for example, introduction of expressible oligonucleotides or nucleic acids encoding polypeptides, other nucleic acid additions, nucleic acid deletions and/or other functional disruption of the microbial organism's genetic material. Such modifications include, for example, nucleotide changes, additions, or deletions in the genomic coding regions and functional fragments thereof, used for heterologous, homologous or both heterologous and homologous expression of polypeptides. Additional modifications include, for example, nucleotide changes, additions, or deletions in the genomic non-coding and/or regulatory regions in which the modifications alter expression of a gene or operon. Exemplary polypeptides include enzymes, proteins, or peptides within a lasso peptide biosynthetic pathway. The terms “non-naturally occurring” or “non-natural” or “unnatural” or “non-native” or “non-canonical” are used to refer to amino acids that are introduced into a polypeptide sequence to modify the properties of the polypeptide.

The term “vector” refers to a substance that is used to carry or include a nucleic acid sequence, including for example, a nucleic acid sequence encoding a lasso precursor peptide, or lasso processing enzymes as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both a lasso core peptide and a lasso cyclase), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product (e.g., a lasso precursor peptide as described herein), and it is further understood that expression levels can be optimized to obtain sufficient expression using methods well known in the art.

The term “encoding nucleic acid” or grammatical equivalents thereof as it is used in reference to nucleic acid molecule refers to a nucleic acid molecule in its native state or when manipulated by methods well known to those skilled in the art that can be transcribed to produce mRNA, which is then translated into a polypeptide and/or a fragment thereof. The antisense strand is the complement of such a nucleic acid molecule, and the encoding sequence can be deduced therefrom.

The term “exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into a microbial organism or into a cell extract for cell-free expression. When used in reference to a biosynthetic activity, the term refers to an activity that is introduced into the host reference organism or into a cell extract for cell-free activity. The source can be, for example, a homologous or heterologous encoding nucleic acid that expresses the referenced activity following introduction into the host microbial organism or into a cell extract for cell-free expression of activity. Therefore, the term “endogenous” refers to a referenced molecule or activity that is present in a microbial host. Similarly, the term when used in reference to expression of an encoding nucleic acid refers to expression of an encoding nucleic acid contained within the microbial organism or into a cell extract. The term “heterologous” refers to a molecule or activity derived from a source other than the referenced species whereas “homologous” refers to a molecule or activity derived from the host microbial organism or organism used to produce a cell-free extract. Accordingly, exogenous expression of an encoding nucleic acid of the invention can utilize either or both a heterologous or homologous encoding nucleic acid.

The term “isolated” when used in reference to a microbial organism or a biosynthetic gene, or a biosynthetic gene cluster, or a protein, or an enzyme, or a peptide, is intended to mean an organism, gene or biosynthetic gene cluster, protein, enzyme, or peptide that is substantially free of at least one component relative to the referenced microbial organism, gene, biosynthetic gene cluster, protein, enzyme, or peptide is found in nature or in its natural habitat. The term includes a microbial organism, gene, biosynthetic gene cluster, protein, enzyme, or peptide that is removed from some or all components as it is found in its natural environment. Therefore, an isolated microbial organism, gene, biosynthetic gene cluster, protein, enzyme, or peptide is partly or completely separated from other substances as it is found in nature or as it is grown, stored or subsisted in non-naturally occurring environments (e.g., laboratories). Specific examples of isolated microbial organisms, genes, biosynthetic gene clusters, proteins, enzymes, or peptides include partially pure microbes, genes, biosynthetic gene clusters, proteins, enzymes, or peptides, substantially pure microbes, genes biosynthetic gene clusters, proteins, enzymes, or peptides, and microbes cultured in a medium that is non-naturally occurring, or genes or biosynthetic gene clusters cloned in non-naturally occurring plasmids, or proteins, enzymes, or peptides purified from other components and substances present their natural environment, including other proteins, enzymes, or peptides.

An “isolated nucleic acid” is a nucleic acid, for example, an RNA, a DNA, or a mixed nucleic acid, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule.

The term “substantially anaerobic” when used in reference to a culture or growth condition is intended to mean that the amount of oxygen is less than about 10% of saturation for dissolved oxygen in liquid media. The term also is intended to include sealed chambers of liquid or solid medium maintained with an atmosphere of less than about 1% oxygen.

The term “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single target-binding site of a binding protein and a single target site of a target molecule is the affinity of the binding protein or functional fragment for that target site. The ratio of dissociation rate (k_{off}) to association rate (kₒₙ) of a binding protein to a monovalent target site (k_{off}/kₒₙ) is the dissociation constant K_{D}, which is inversely related to affinity. The lower the K_{D} value, the higher the affinity of the antibody. The value of K_{D} varies for different complexes of lasso peptides or target proteins depends on both kₒₙ and k_{off}. The dissociation constant K_{D} for a binding protein (e.g., a lasso peptide) provided herein can be determined using any method provided herein or any other method well known to those skilled in the art. The affinity at one binding site does not always reflect the true strength of the interaction between a binding protein and the target molecule. When complex target molecule containing multiple, repeating target sites, such as a polyvalent target protein, come in contact with lasso peptides containing multiple target binding sites, the interaction of the lasso peptide with the target protein at one site will increase the probability of a reaction at a second site.

The term “binding affinity” generally refers to the strength of the sum total of noncovalent interactions between a single binding site of a molecule (e.g., a binding protein such as a lasso peptide) and its binding partner (e.g., a target protein). Unless indicated otherwise, as used herein, “binding affinity” refers to intrinsic binding affinity which reflects a 1:1 interaction between members of a binding pair (e.g., lasso peptide and target protein). The affinity of a binding molecule X for its binding partner Y can generally be represented by the dissociation constant (K_{D}). Affinity can be measured by common methods known in the art, including those described herein. Low-affinity lasso peptides generally bind target proteins slowly and tend to dissociate readily, whereas high-affinity lasso peptides generally bind target proteins faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the lasso peptide of interest and its target protein. The K_{D} or K_{D} value may also be measured by using surface plasmon resonance assays by Biacore®, using, for example, a Biacore® ™-2000 or a Biacore® ™-3000, or by biolayer interferometry using, for example, the Octet® QK384 system. An “on-rate” or “rate of association” or “association rate” or “kₒₙ” may also be determined with the same surface plasmon resonance or biolayer interferometry techniques described above using, for example, a Biacore™-2000 or a Biacore® ™-3000, or the Octet® QK384 system.

The terms “target molecule” and “target protein” are used interchangeably herein and refer to a protein with which a lasso peptide binds under a physiological condition that mimics the native environment where the protein is isolated or derived from. As used herein, the target molecule is a cell surface protein or an extracellularly secreted protein. “Cell surface protein” is a term of art, and is used herein to refer to any protein that is known by the skilled person as a cell surface protein, and including those with any form of post-translational modifications, such as glycosylation, phosphorylation, lipidation, etc. In various embodiments, a cell surface protein can be a peptide or protein that has at least one part exposed to the extracellular environment, while embedded in or span the lipid layer of the cell membrane, or associated with a molecule integrated in the lipid layer. In some embodiments, a target molecule can be endothelin B receptor (ETBR) expressed on the surface of a neoplastic cell or an endothelial cell. In certain embodiments, a target molecule mediates one or more cellular activities (e.g., through a cellular signaling pathway), and as a result of the binding of a lasso peptide to the target molecule, the cellular activities are modulated.

The term “target site” as used herein refers to the amino acid residue or the group of amino acid residues with which a particular lasso peptide interacts to form the binding with the target molecule. According to the present disclosure, different lasso peptides may bind to different target sites or compete for binding with the same target site of a target molecule. In some embodiments, a lasso peptide specifically binds to a target molecule or a target site thereof.

A lasso peptide which “binds a target molecule of interest” is one that binds the target molecule with sufficient affinity such that the lasso peptide is useful, for example, as a diagnostic or therapeutic agent in targeting a cell or tissue expressing the target molecule, and does not significantly cross-react with other molecules. In such embodiments, the extent of binding of the lasso peptide to a “non-target” molecule will be less than about 10% of the binding of the lasso peptide to its particular target molecule, for example, as determined by fluorescence activated cell sorting (FACS) analysis or RIA.

With regard to the binding of a lasso peptide to a target molecule, the term “specific binding,” “specifically binds to,” or “is specific for” a particular polypeptide or an fragment on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding,” “specifically binds to,” or “is specific for” a particular polypeptide or a fragment on a particular polypeptide target as used herein refers to binding where a molecule binds to a particular polypeptide or fragment on a particular polypeptide without substantially binding to any other polypeptide or polypeptide fragment. In certain embodiments, a lasso peptide that binds to a target molecule has a dissociation constant (K_{D}) of less than or equal to 100 μM, 80 μM, 50 μM, 25 μM, 10 μM, 5 μM, 1 μM, 900 nM, 800 nM, 700 nM, 600 nM, 500 nM, 400 nM, 300 nM, 200 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, 0.1 nM, 0.09 nM, 0.08 nM, 0.07 nM, 0.06 nM, 0.05 nM, 0.04 nM, 0.03 nM, 0.02 nM, or 0.01 nM.

In the context of the present disclosure, a target protein is said to specifically bind to a lasso peptide, for example, when the dissociation constant (K_{D}) is ≤10⁻⁷M. In some embodiments, the lasso peptides specifically bind to a target protein with a K_{D} of from about 10⁻⁷ M to about 10⁻¹²M. In certain embodiments, the lasso peptides specifically bind to a target protein with high affinity when the K_{D} is ≤10⁻⁸M or K_{D} is ≤10⁻⁹M. In one embodiment, the lasso peptides may specifically bind to a purified human target protein with a K_{D} of from 1×10⁻⁹ M to 10×10⁻⁹ M as measured by Biacore®. In another embodiment, the lasso peptides may specifically bind to a purified human target protein with a K_{D} of from 0.1×10⁻⁹M to 1×10⁻⁹M as measured by KinExA™ (Sapidyne, Boise, Id.). In yet another embodiment, the lasso peptides specifically bind to a target protein expressed on cells with a K_{D} of from 0.1×10⁻⁹M to 10×10⁻⁹ M. In certain embodiments, the lasso peptides specifically bind to a human target protein expressed on cells with a K_{D} of from 0.1×10⁻⁹ M to 1×10⁻⁹M. In some embodiments, the lasso peptides specifically bind to a human target protein expressed on cells with a K_{D} of 1×10⁻⁹M to 10×10⁻⁹M. In certain embodiments, the lasso peptides specifically bind to a human target protein expressed on cells with a K_{D} of about 0.1×10⁻⁹M, about 0.5×10⁻⁹M, about 1×10⁻⁹ M, about 5×10⁻⁹M, about 10×10⁻⁹M, or any range or interval thereof. In still another embodiment, the lasso peptides specifically bind to a non-human target protein expressed on cells with a K_{D} of 0.1×10⁻⁹M to 10×10⁻⁹M. In certain embodiments, the lasso peptides specifically bind to a non-human target protein expressed on cells with a K_{D} of from 0.1×10⁻⁹M to 1×10⁻⁹M. In some embodiments, the lasso peptides specifically bind to a non-human target protein expressed on cells with a K_{D} of 1×10⁻⁹ M to 10×10⁻⁹M. In certain embodiments, the lasso peptides specifically bind to a non-human target protein expressed on cells with a K_{D} of about 0.1×10⁻⁹M, about 0.5×10⁻⁹M, about 1×10⁻⁹ M, about 5×10⁻⁹M, about 10×10⁻⁹M, or any range or interval thereof.

With regard to the binding of a lasso peptide to a target molecule, the term “preferential binding” or “preferentially binds to” a particular polypeptide or an fragment on a particular target molecule with respect to a reference molecule means binding of the target molecule is measurably higher than binding of the reference molecule, while the reference molecule may or may not also bind to the lasso peptide. For example, in some embodiments, a lasso peptide preferentially binds to endothelin B receptor (ETBR) over endothelin A receptor (ETAR). Preferential binding can be determined, for example, by determining the binding affinity. For example, a lasso peptide that preferentially binds to a target molecule over a reference molecule can bind to the target molecule with a K_{D} less than the K_{D} exhibited relative to the reference molecule. In some embodiments, the lasso peptide preferentially binds a target molecule with a K_{D} less than half of the K_{D} exhibited relative to the reference molecule. In some embodiments, the lasso peptide preferentially binds a target molecule with a K_{D} at least 10 times less than the K_{D} exhibited relative to the reference molecule. In some embodiments, the lasso peptide preferentially binds a target molecule with a K_{D} with K_{D} that is about 75%, about 50%, about 25%, about 10%, about 5%, about 2.5%, or about 1% of the K_{D} exhibited relative to the reference molecule. In some embodiments, the ratio between the K_{D} exhibited by the lasso peptide when binding to the reference molecule and the K_{D} exhibited when binding to the target molecule is at least 2 fold, at least 3 fold, at least 4 fold, at least 5 fold, at least 10 fold, at least 20 fold, at least 100 fold, at least 500 fold, at least 10³ fold, at least 10⁴ fold, or at least 10⁵ fold.

A lasso peptide that specifically or preferentially binds to a target protein can be identified, for example, by immunoassays (e.g., ELISA, fluorescent immunosorbent assay, chemiluminescence immune assay, radioimmunoassay (MA), enzyme multiplied immunoassay, solid phase radioimmunoassay (SPRIA), a surface plasmon resonance (SPR) assay (e.g., Biacore®), a fluorescence polarization assay, a fluorescence resonance energy transfer (FRET) assay, Dot-blot assay, fluorescence activated cell sorting (FACS) assay, or other techniques known to those of skill in the art. A lasso peptide binds specifically to a target protein when it binds to the target protein with higher affinity than to any cross-reactive target molecule as determined using experimental techniques, such as radioimmunoassays (RIA) and enzyme linked immunosorbent assays (ELISAs). Typically a specific or selective reaction will be at least twice background signal or noise and may be more than 10 times background.

The term “compete” when used in the context of lasso peptides (e.g., a lasso peptide and other binding proteins that bind to and compete for the same target molecule or target site on the target molecule) means competition as determined by an assay in which the lasso peptide (or binding fragment) thereof under study prevents or inhibits the specific binding of a reference molecule (e.g., a reference ligand of the target molecule) to a common target molecule. Numerous types of competitive binding assays can be used to determine if a test lasso peptide competes with a reference ligand for binding to a target molecule. Examples of assays that can be employed include solid phase direct or indirect RIA, solid phase direct or indirect enzyme immunoassay (EIA), sandwich competition assay (see, e.g., Stahli et al., 1983, Methods in Enzymology 9:242-53), solid phase direct biotin-avidin EIA (see, e.g., Kirkland et al., 1986, J. Immunol. 137:3614-19), solid phase direct labeled assay, solid phase direct labeled sandwich assay (see, e.g., Harlow and Lane, Antibodies, A Laboratory Manual (1988)), solid phase direct label RIA using I-125 label (see, e.g., Morel et al., 1988, Mol. Immunol. 25:7-15), and direct labeled RIA (Moldenhauer et al., 1990, Scand. J. Immunol. 32:77-82). Typically, such an assay involves the use of a purified target molecule bound to a solid surface, or cells bearing either of an unlabeled test target-binding lasso peptide or a labeled reference target-binding protein (e.g., reference target-binding ligand). Competitive inhibition may be measured by determining the amount of label bound to the solid surface in the presence of the test target-binding lasso peptide. Usually the test target-binding protein is present in excess. Target-binding lasso peptides identified by competition assay (e.g., competing lasso peptides) include lasso peptides binding to the same target site as the reference and lasso peptides binding to an adjacent target site sufficiently proximal to the target site bound by the reference for steric hindrance to occur. Additional details regarding methods for determining competitive binding are described herein. Usually, when a competing lasso peptide is present in excess, it will inhibit specific binding of a reference to a common target molecule by at least 30%, for example 40%, 45%, 50%, 55%, 60%, 65%, 70%, or 75%. In some instance, binding is inhibited by at least 80%, 85%, 90%, 95%, 96%, 97%, 98%, 99%, or more.

The term “blocking” lasso peptide or an “antagonist” lasso peptide is one which inhibits or reduces biological activity of the target molecule it binds. For example, blocking lasso peptide or antagonist lasso peptide may substantially or completely inhibit the biological activity of the target molecule.

The term “inhibition” or “inhibit,” when used herein, refers to partial (such as, 1%, 2%, 5%, 10%, 20%, 25%, 50%, 75%, 90%, 95%, 99%) or complete (i.e., 100%) inhibition. The term “IC₅₀” refers an amount, concentration, or dosage of a compound that results in 50% inhibition of a maximal response in an assay that measures such response. The term “EC₅₀” refers an amount, concentration, or dosage of a compound that results in for 50% of a maximal response in an assay that measures such response.

The term “attenuate,” “attenuation,” or “attenuated,” when used herein, refers to partial (such as, 1%, 2%, 5%, 10%, 20%, 25%, 50%, 75%, 90%, 95%, 99%) or complete (i.e., 100%) reduction in a property, activity, effect, or value.

With regard to inhibition or attenuation of a target molecule by a lasso peptide, the term “selective inhibition of,” “selectively inhibits,” “selective attenuation of,” or “selectively attenuates” a target molecule or a signaling pathway mediated by a target molecule means inhibition of the target molecule activity is measurably stronger than inhibition of a reference molecule activity. For example, in some embodiments, a lasso peptide selectively inhibits endothelin B receptor (ETBR) over endothelin A receptor (ETAR). Selective inhibition can be determined, for example, by determining the IC₅₀ value. For example, a lasso peptide that selectively inhibits or attenuates a target molecule can exhibit an IC₅₀ value less than the IC₅₀ exhibited relative to a reference molecule. In some embodiments, the lasso peptide selectively inhibits or attenuates a target molecule with an IC₅₀ less than half of the IC₅₀ exhibited relative to the reference molecule. In some embodiments, the lasso peptide selectively inhibits or attenuates a target molecule with an IC₅₀ that is about 75%, about 50%, about 25%, about 10%, about 5%, about 2.5%, or about 1% of the IC₅₀ exhibited relative to the reference molecule. In some embodiments, the ratio between the IC₅₀ exhibited by the lasso peptide with respect to the reference molecule and the IC₅₀ exhibited with respect to the target molecule is at least 2 fold, at least 3 fold, at least 4 fold, at least 5 fold, at least 10 fold, at least 20 fold, at least 100 fold, at least 500 fold, at least 10³ fold, at least 10⁴ fold, or at least 10⁵ fold.

The phrase “substantially similar” or “substantially the same” denotes a sufficiently high degree of similarity between two numeric values (e.g., one associated with a lasso peptide of the present disclosure and the other associated with a reference ligand) such that one of skill in the art would consider the difference between the two values to be of little or no biological and/or statistical significance within the context of the biological characteristic measured by the values (e.g., K_{D} values). For example, the difference between the two values may be less than about 50%, less than about 40%, less than about 30%, less than about 20%, less than about 10%, or less than about 5%, as a function of the value for the reference ligand.

The phrase “substantially increased,” “substantially reduced,” or “substantially different,” as used herein, denotes a sufficiently high degree of difference between two numeric values (e.g., one associated with a lasso peptide of the present disclosure and the other associated with a reference ligand) such that one of skill in the art would consider the difference between the two values to be of statistical significance within the context of the biological characteristic measured by the values. For example, the difference between said two values can be greater than about 10%, greater than about 20%, greater than about 30%, greater than about 40%, or greater than about 50%, as a function of the value for the reference ligand.

The term “assaying” is meant the creation of experimental conditions and the gathering of data regarding a particular result of the exposure to specific experimental conditions. For example, enzymes can be assayed based on their ability to act upon a detectable substrate. A lasso peptide can be assayed based on its ability to bind to a particular target molecule or molecules.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In one embodiment, the subject is a mammal (e.g., a human) having an endothelin B receptor (ETBR)-mediated proliferative disease (e.g. cancer), disorder, or condition. In another embodiment, the subject is a mammal (e.g., a human) at risk of developing an endothelin B Receptor (ETBR)-mediated proliferative disease (e.g. cancer), disorder, or condition.

The term “proliferative” or “neoplastic” disease or condition refers to any physiological condition in animals that is characterized by uncontrolled, abnormal growth of cells, referred to as “neoplastic cells.” Neoplastic cells, as used herein, can be malignant or benign, and includes both solid tumors as well as hematologic tumors and/or malignancies. Non-limiting examples of proliferative diseases that can be prevented, treated or managed with the methods and compositions described herein include those mediated by endothelin B receptor activity, such as breast cancer, pancreatic cancer (e.g., pancreatic adenocarcinoma), hepatocellular carcinoma, prostate cancer, ovarian cancer, gastric cancer, glioblastoma, melanoma, cancer of the head and neck, colorectal cancer, bladder cancer, vulvar cancer, esophageal squamous cell carcinoma, renal cancer (e.g., clear-cell renal cell carcinoma), cervical cancer, salivary gland carcinoma, lung cancer (e.g., non-small cell lung cancer and small-cell lung cancer), multiple myeloma, or Kaposi's sarcoma. In particular embodiments, the proliferative disease being treated is melanoma. In particular embodiments, the proliferative disease being treated is ovarian cancer.

The term “microenvironment” a neoplastic cell or neoplastic cells or “neoplastic microenvironment” refers to elements of the neoplasia milieu that creates a structural and/or functional environment for the neoplastic process to survive, expand, and/or spread. As a non-limiting example, a neoplastic microenvironment is constituted by the cells, molecules, extracellular matrix and/or blood vessels that surround and/or feed one or more neoplastic cells, such as a solid tumor. In certain embodiments, the neoplastic disease is a solid tumor. Exemplary cells or tissue within the tumor microenvironment include, but are not limited to, tumor vasculature, tumor infiltrating lymphocytes, fibroblast reticular cells, endothelial progenitor cells (EPC), cancer-associated fibroblasts, pericytes, other stromal cells, components of the extracellular matrix (ECM), dendritic cells, antigen presenting cells, T-cells, regulatory T-cells, macrophages, neutrophils, and other immune cells located proximal to a tumor. Exemplary cellular functions affecting the tumor microenvironment include, but are not limited to, production of cytokines and/or chemokines, response to cytokines, antigen processing and presentation of peptide antigen, regulation of leukocyte chemotaxis and migration, regulation of gene expression, complement activation, regulation of signaling pathways, cell-mediated cytotoxicity, cell-mediated immunity, humoral immune responses, and innate immune responses, etc.

The term “vasculature” refers to blood and lymph vessels that carry whole blood and lymphatic fluids. The term “tumor vasculature” refers to blood or lymph vessels that feed into a tumor.

The terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The term “therapy” refers to any protocol, method, and/or agent that can be used in the prevention, management, treatment, and/or amelioration of an endothelin B receptor-mediated proliferative disease (e.g., cancer), disorder, or condition. In certain embodiments, the terms “therapies” and “therapy” refer to a biological therapy, supportive therapy, and/or other therapies useful in the prevention, management, treatment, and/or amelioration of a endothelin B receptor-mediated proliferative disease (e.g., cancer), disorder, or condition, known to one of skill in the art such as medical personnel.

The term “therapeutic agent” refers to any agent that can be used in treating, preventing, or alleviating a disease, disorder, or condition, including in the treatment, prevention, or alleviation of one or more symptoms of an endothelin B receptor-mediated proliferative disease (e.g., cancer), disorder, or condition and/or a symptom related thereto. In certain embodiments, a therapeutic agent refers to a lasso peptide as described herein.

The term “therapeutically effective amount” as used herein refers to the amount of an agent (e.g., a lasso peptide provided herein or any other agent described herein) that is sufficient to reduce and/or ameliorate the severity and/or duration of a given disease, disorder, or condition, and/or a symptom related thereto (e.g., cancer). A “therapeutically effective amount” of a substance/molecule/agent of the present disclosure (e.g., a lasso peptide) may vary according to factors such as the disease state, age, sex, and weight of the individual, and the ability of the substance/molecule/agent to elicit a desired response in the individual. A therapeutically effective amount encompasses an amount in which any toxic or detrimental effects of the substance/molecule/agent are outweighed by the therapeutically beneficial effects. In certain embodiments, the term “therapeutically effective amount” refers to an amount of a lasso peptide or other agent (e.g., drug) effective to “treat” a disease, disorder, or condition, in a subject or mammal.

The terms “prevent,” “preventing,” and “prevention” refer to delaying or reducing the likelihood of the onset (or recurrence) of a disease, disorder, condition, or associated symptom(s) (e.g., cancer).

The term “prophylactic agent” refers to any agent that can totally or partially inhibit the development, recurrence, onset, or spread of an endothelin B receptor-mediated proliferative disease (e.g., cancer) and/or symptom related thereto in a subject. In certain embodiments, the term “prophylactic agent” refers to a lasso peptide as described herein.

The term “prophylactically effective amount” refers to an amount of a pharmaceutical composition that, when administered to a subject, will have the intended prophylactic effect, e.g., preventing, delaying, or reducing the likelihood of the onset (or reoccurrence) of a disease, disorder, condition, or associated symptom(s) (e.g., cancer). Typically, but not necessarily, since a prophylactic dose is used in subjects prior to or at an earlier stage of a disease, disorder, or condition, a prophylactically effective amount may be less than a therapeutically effective amount. The full therapeutic or prophylactic effect does not necessarily occur by administration of one dose, and may occur only after administration of a series of doses. Thus, a therapeutically or prophylactically effective amount may be administered in one or more administrations.

The terms “manage,” “managing,” and “management” refer to the beneficial effects that a subject derives from a therapy (e.g., a prophylactic or therapeutic agent), which does not result in a cure of the disease. In certain embodiments, a subject is administered one or more therapies (e.g., prophylactic or therapeutic agents, such as a lasso peptide provided herein) to “manage” an endothelin B receptor-mediated proliferative disease (e.g., cancer), one or more symptoms thereof, so as to prevent the progression or worsening of the disease.

The term “modulating” or “modulate” as used herein refers to an effect of altering a biological activity (i.e. increasing or decreasing the activity), especially a biological activity associated with a particular biomolecule such as a, enzyme or cell surface receptor. For example, an inhibitor of a particular biomolecule modulates the activity of that biomolecule, e.g., an enzyme, by decreasing the activity of the biomolecule, such as an enzyme. Such activity is typically indicated in terms of an inhibitory concentration (IC₅₀) of the compound for an inhibitor with respect to, for example, an enzyme or a cell surface receptor.

The term “side effects” encompasses unwanted and/or adverse effects of a therapy (e.g., a prophylactic or therapeutic agent). Unwanted effects are not necessarily adverse. An adverse effect from a therapy (e.g., a prophylactic or therapeutic agent) might be harmful, uncomfortable, or risky. Examples of side effects include, diarrhea, cough, gastroenteritis, wheezing, nausea, vomiting, anorexia, abdominal cramping, fever, pain, loss of body weight, dehydration, alopecia, dyspenea, insomnia, dizziness, mucositis, nerve and muscle effects, fatigue, dry mouth, loss of appetite, rashes or swellings at the site of administration, flu-like symptoms such as fever, chills, and fatigue, digestive tract problems, and allergic reactions. Additional undesired effects experienced by patients are numerous and known in the art. Many are described in Physician's Desk Reference (68th ed. 2014).

The term “administer” or “administration” refers to the act of injecting or otherwise physically delivering a substance as it exists outside the body (e.g., a lasso peptide as described herein) into a patient, such as by mucosal, intradermal, intravenous, intramuscular delivery, and/or any other method of physical delivery described herein or known in the art. When a disease, disorder, condition, or a symptom thereof, is being treated, administration of the substance typically occurs after the onset of the disease, disorder, condition, or symptoms thereof. When a disease, disorder, condition, or symptoms thereof, are being prevented, administration of the substance typically occurs before the onset of the disease, disorder, condition, or symptoms thereof.

The term “co-administration” and its grammatical variants as used herein refer to the simultaneous or sequential administration of at least two therapeutic agents according to the present disclosure. For example, a lasso peptide as disclosed herein can be administered with another therapeutic agent simultaneously or sequentially in separate unit dosage forms or together in a single unit dosage form.

The term “chronic” administration refers to administration of the agent(s) in a continuous mode (e.g., for a period of time such as days, weeks, months, or years) as opposed to an acute mode, so as to maintain the initial therapeutic effect (activity) for an extended period of time. “Intermittent” administration is treatment that is not consecutively done without interruption, but rather is cyclic in nature.

The term “effective amount” as used herein generally refers to an amount sufficient to reduce the severity and/or frequency of symptoms, eliminate the symptoms and/or underlying cause, prevent the occurrence of symptoms and/or their underlying cause, and/or improve or remediate the damage that results from or is associated with a disease, disorder, or condition, including, for example, cancer. In some embodiments, the effective amount is a therapeutically effective amount or a prophylactically effective amount.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

The term “carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. The term “carrier” can also refer to a diluent, adjuvant (e.g., Freund's adjuvant (complete or incomplete)), excipient, or vehicle. Such carriers, including pharmaceutical carriers, can be sterile liquids, such as water and oils, including those of petroleum, animal, vegetable, or synthetic origin, such as peanut oil, soybean oil, mineral oil, sesame oil, and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard carriers such as pharmaceutical grades of mannitol, lactose, starch, magnesium stearate, sodium saccharine, cellulose, magnesium carbonate, etc. Examples of suitable pharmaceutical carriers are described in Remington and Gennaro, Remington's Pharmaceutical Sciences (18th ed. 1990). Compositions, including pharmaceutical compounds, may contain a lasso peptide, for example, in isolated or purified form, together with a suitable amount of carriers.

The term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.), and polyols (e.g., mannitol, sorbitol, etc.). See, also, Remington and Gennaro, Remington's Pharmaceutical Sciences (18th ed. 1990), which is hereby incorporated by reference in its entirety.

The term “contacting” and its grammatical variations, when used in reference to two or more components, refers to any process whereby the approach, proximity, mixture or commingling of the referenced components is promoted or achieved without necessarily requiring physical contact of such components, and includes mixing of solutions containing any one or more of the referenced components with each other. The referenced components may be contacted in any particular order or combination and the particular order of recitation of components is not limiting. For example, “contacting A with B and C” encompasses embodiments where A is first contacted with B then C, as well as embodiments where C is contacted with A then B, as well as embodiments where a mixture of A and C is contacted with B, and the like. Furthermore, such contacting does not necessarily require that the end result of the contacting process be a mixture including all of the referenced components, as long as at some point during the contacting process all of the referenced components are simultaneously present or simultaneously included in the same mixture or solution.

The term “partially” means that something takes place, as a function or activity, to provide the expected outcome or result in part and to a limited extent, not to the fullest extent. For example, if a lasso peptide is partially purified, the lasso peptide is isolated and purification steps afford the lasso peptide at purity level that is greater than about 20% and less than about 90%.

### 5.3 Endothelin Receptor Antagonism

In certain aspects of the present disclosure, provided herein are endothelin B receptor (ETBR) antagonistic compounds and their uses for managing, preventing, and/or treating an endothelin B receptor (ETBR)-mediated proliferative disease in a subject.

Endothelin (ET) receptors are transmembrane G protein-coupled receptors (GPCRs) normally expressed on the surface of endothelial cells lining the inner wall of blood and lymphatic vessels. Two main receptors, endothelin receptor type A (ETAR) and endothelin receptor type B (ETBR), regulate normal vascular function by binding to one of three cognate endothelin ligands, comprised of the 21-amino acid peptides endothelin-1 (ET-1), endothelin-2 (ET-2), or endothelin-3 (ET-3). The vascular endothelium is an abundant source of the components of the endothelin axis; however, they also are expressed to varying extents by leukocytes, smooth muscle cells, mesangial cells, cardiac myocytes, and astrocytes. In humans, ETAR is located in the vasculature and is mostly expressed by cells of the vascular smooth muscle lineage. In these cells, binding of ET-1 to ETAR mainly induces vasoconstriction and cell proliferation (Maguire, J. J., Davenport, A. P., Endothelium receptors and their antagonists, Sem. Nephrology, 2015, 35(2), 125-136).

ETBR in the vasculature is mostly expressed by endothelial cells. In these cells, binding of ETs to ETBR induces vasodilatation, bronchoconstriction, and cell proliferation. The human kidney is unusual among the peripheral organs in expressing a high density of ETBR. The renal vascular endothelium only expresses the ETBR subtype and ET-1 acts in an autocrine or paracrine manner to release vasodilators. Endothelial ETBR in kidney, as well as liver and lungs, appears to play a critical role in scavenging ET-1 from the plasma. The third major function for ET-1 is activation of ETBR in medullary epithelial cells to reduce salt and water reabsorption.

ET-1 can be induced in endothelial cells by many factors including mechanical stimulation, various hormones, and proinflammatory cytokines. Its production is inhibited by nitric oxide (NO), cyclic nucleotides, prostacyclin, and atrial natriuretic peptide (ANP). ET-1 also stimulates cardiac contraction and the growth of cardiac myocytes, regulates the release of vasoactive substances, and stimulates smooth muscle cell mitogenesis. ET-1 may control inflammatory responses by promoting the adhesion and migration of neutrophils and by stimulating the production of proinflammatory cytokines.

ETAR and ETBR are GPCRs that transmit signals via heterotrimeric guanine nucleotide-binding G proteins, which are composed of α-, β-, and γ-subunits on the inner membrane surface of the cells, and are key determinants of many signaling processes, including cell proliferation, apoptosis, survival, contraction, migration, and/or differentiation (Cabrera-Vera, T. M., et al., Insights into G Protein Structure, Function, and Regulation. Endocr. Rev. 2003, 24, 765-781). GPCR ligands interact with many downstream effectors, including adenyl cyclases, phosphodiesterases, phospholipases, tyrosine kinases, and ion channels. The duration of the signal is modulated by the activity of the multiple GPCR-mediated signaling pathways, leading to diverse biological responses. At physiological concentrations, ET-1 and ET-2, but not ET-3, bind to ETAR receptors with comparable affinity (K_{D}(ET-1)=K_{D}(ET-2)≈20-60 pM, K_{D}(ET-3)≈6500 pM), whereas all three ET ligands bind ETBR receptors with similar affinity (K_{D}(ET-1)=K_{D}(ET-2)=K_{D}(ET-3) 15 pM).

Endothelin-induced intracellular signaling transduced by activated ETAR and ETBR, which together control vascular homeostasis by balancing vasoconstriction, vasodilation, angiogenesis, and lymphangiogenesis (Vignon-Zellweger, N., et al., Endothelin and endothelin receptors in the renal and cardiovascular systems, Life Sciences, 2012, 91, 490-500). Consistent with these roles, extensive early work revealed the various roles of the ET system in cardiovascular and renal disorders (Tomobe et al., Effects of endothelin on the renal artery from spontaneously hypertensive and Wistar Kyoto rats, Eur. J. Pharmacol., 1988, 152(3): 373-374; Lehrke et al., Renal endothelin-1 and endothelin receptor type B expression in glomerular diseases with proteinuria. J Am Soc Nephrol., 2001, 12(11): 2321-2329; Feldstein et al., Role of endothelins in hypertension. Am J Ther., 2007, 14(2): 147-153; Iglarz M et al., Mechanisms of ET-1-induced endothelial dysfunction, J Cardiovasc Pharmacol., 2007, 50(6): 621-628) and the ET axis was targeted by therapeutic intervention largely for these diseases.

ETBR activation specifically mediates the release of relaxing factors such as nitric oxide, prostacyclin, and endothelium-derived hyperpolarizing factor, increases in [Ca²⁺]ᵢ, protein kinase C, mitogen-activated protein kinase, and other pathways involved in vascular contraction and cell growth (Mazzuca, M. Q., Khalil, R. A. Vascular endothelin receptor type B: structure, function and dysregulation in vascular disease, Biochem Pharmacol. 2012; 84(2): 147-162). ETBR has been shown to be overexpressed in a variety of cancers and expression levels correlate with low survival and poor prognosis (Rosano, L., Bagnato, A., Endothelin therapeutics in cancer: Where are we? Am J Physiol Regul Integr Comp Physiol, 2016, 310: R469-R475; Bagnato, A., et al, Role of the endothelin axis and its antagonists in the treatment of cancer, Brit. Pharmacol., 2011, 163, 220-233). ETBR is overexpressed on the surface of tumor cells and promotes growth, proliferation, and metastasis of many tumor types, including esophageal squamous cell carcinoma (Ishimoto, S., et al., Role of endothelin receptor signalling in squamous cell carcinoma, Int. J Oncology, 2012, 40, 1011-1019; Tanaka, T., et al., Endothelin B receptor expression correlates with tumour angiogenesis and prognosis in oesophageal squamous cell carcinoma, Brit. J. Cancer, 2014, 110, 1027-1033), breast cancer (Grimshaw, M. J., et al., A Role for Endothelin-2 and Its Receptors in Breast Tumor Cell Invasion, Cancer Res., 2004, 64, 2461-2468; Wulfing, P., et al, Expression of endothelin-1, endothelin-A, and endothelin-B receptor in human breast cancer and correlation with long-term follow-up, Clin. Cancer Res., 2003, 9, 4125-4131), glioblastoma (Vasaiker, S., et al., Overexpression of endothelin B receptor in glioblastoma: a prognostic marker and therapeutic target? BMC Cancer, 2018, 18: 154), oligodendroglioma (Wan, X., et al., Role of endothelin B receptor in oligodendroglioma proliferation and survival, in vitro and in vivo evidence, Mol. Med. Rep., 2014, 9: 229-234), bladder cancer (Wulfing, C., et al., Expression of the endothelin axis in bladder cancer: relationship to clinicopathologic parameters and long-term survival, Eur. Urol., 2005, 47(5), 593-600), head and neck cancer (Awano, S., et al., Endothelin system in oral squamous carcinoma cells: Specific siRNA targeting of ECE-1 blocks cell proliferation, Int. J. Cancer, 2006, 118, 1645-1652), vulvar cancer (Eltze, E., et al., Expression and prognostic relevance of endothelin-B receptor in vulvar cancer, Oncology Rep., 2007, 18, 305-311), clear-cell renal cell carcinoma (Wuttig, D., et al., CD31, EDNRB and TSPAN7 are promising prognostic markers in clear-cell renal cell carcinoma revealed by genome-wide expression analyses of primary tumors and metastases, Int. J. Cancer, 2012, 131, E693-E704), multiple myeloma (Russignan, A., et al., Endothelin-1 receptor blockade as a new possible therapeutic approach in multiple myeloma, British Journal of Haematology, 2017, 178, 781-793), pancreatic adenocarcinoma (Cook, N., et al., Endothelin-1 and endothelin B receptor expression in pancreatic adenocarcinoma. J. Clin. Pathol., 2015, 68(4), 309-313), and Kaposi's sarcoma (Rosano, R., et al., Endothelin receptor blockade inhibits molecular effectors of Kaposi's sarcoma cell invasion and tumor growth in vivo, Am. J. Pathology, 2003, 163(2), 753-762).

Without being bound by the theory, it is contemplated that in addition to tumor expression, ETBR is upregulated in the tumor microenvironment on the endothelial cells of tumor vasculature. For example, a method involving laser-capture microdissection was employed to conclusively demonstrate that ETBR is highly overexpressed in the surface of endothelial cells of the vasculature of ovarian cancer tumors and that ETBR overexpression was strongly correlated with low overall patient survival (Buckanovich, R. J., et al., Endothelin B receptor mediates the endothelial barrier to T cell homing to tumors and disables immune therapy, Nature Med., 2008; 14(1): 28-36). Expression of ETBR in tumor vasculature endothelial cells can reduce the level of intraepithelial tumor infiltrating leukocytes (TILs) in the tumor microenvironment via a mechanism that involved significant decrease of the intercellular adhesion molecule ICAM-1, which is required for leukocyte migration through the vasculature to the tumor (Buckanovich, R. J., et al., Endothelin B receptor mediates the endothelial barrier to T cell homing to tumors and disables immune therapy, Nature Med., 2008; 14(1): 28-36). Further, ETAR is required for the high expression of endothelial ICAM-1 and other adhesion molecules that are important for TIL migration (Coffman, L., et al., Endothelin receptor A is required for the recruitment of antitumor T cells and modulates chemotherapy induction of cancer stem cells. Cancer Biol. Ther., 2013, 14(2), 184-192). Thus, in some embodiments, a highly selective ETBR antagonist is used.

Moreover, neutrophils are involved in tumor progression and metastasis (Shaul, M. E., Fridlender, C. G., Tumour-associated neutrophils in patients with cancer, Nature Rev. Clin. Oncol., 2019, 16, 601-620) and ETBR antagonism can block pro-tumor neutrophil migration (Zarpelon, A. C., et al., Endothelin-1 induces neutrophil recruitment in adaptive inflammation via TNFα and CXCL1/CXCR2 in mice. Canadian J. Physiol. and Pharmacol., 2012, 90(2), 187-199). Without being bound by the theory, it is contemplated that ETBR antagonist can enhance the efficacy of immunotherapy drugs.

### 5.4 Compositions and Methods of Making the Same

5.4.1 Endothelin Receptor Antagonistic Lasso Peptides.

In a first aspect of the present disclosure, provided herein are endothelin receptor antagonistic lasso peptides. Particularly, thirty two (32) lasso peptides having different amino acid sequences are provided, and are referred to Lasso 1-17 and 42-56, respectively in this application. Table 1 summarizes the core peptide sequences and the precursor peptide sequences corresponding to Lassos 1-17. In some embodiments, Lasso 1-17 and 42-56 differ from their respective core peptides having SEQ ID NOS:1-17 and 42-56 in that Lasso 1-17 and 42-56 have the lariat conformation, while the core peptides do not possess such secondary structure. In some embodiments, any one of Lasso 1-17 and 42-56 is G1-D9 cyclized.

TABLE 1

| | Lasso Core Peptide | |
|---|---|---|
| | A.A. Sequence | Lasso Precursor Peptide A.A. Sequence |
| Lasso | (SEQ ID NO:) | (SEQ ID NO:) |
| 1 | GNWHGTAPDWFFN | MTEQSEQTPAEYIPPMLVEVGEFTEDTLGNWHGT |
| | YYW (1) | APDWFFNYYW (18) |
| 2 | GNWHGTAPDWFFN | MTEQSEQTPAEYIPPMLVEVGEFTEDTLGNWHGT |
| | YYW (1) | APDWFFNYYW (18) |
| | or | or |
| | GNWHGTAPDWFFN | MTEQSEQTPAEYIPPMLVEVGEFTEDTLGNWHGT |
| | YYW-7-OH (2) | APDWFFNYYW-7-OH (19) |
| 3 | GNWHGTSPDWFFN | MTEQSEQTPAEYIPPMLVEVGEFTEDTLGNWHGT |
| | YYW (3) | SPDWFFNYYW (20) |
| 4 | GNWHGTSPDWFFN | MTEQSEQTPAEYIPPMLVEVGEFTEDTLGNWHGT |
| | YYW (3) | SPDWFFNYYW (20) |
| | or | or |
| | GNWHGTSPDWFFN | MTEQSEQTPAEYIPPMLVEVGEFTEDTLGNWHGT |
| | YYW-7-OH (4) | SPDWFFNYYW-7-OH (21) |
| 5 | GNWHGTSPDWFFN | MTEQSEQTPAEYIPPMLVEVGEFTEDTLGNWHGT |
| | YY (5) | SPDWFFNYY (22) |
| 6 | GNWHGTSPDWFFN | MTEQSEQTPAEYIPPMLVEVGEFTEDTLGNWHGT |
| | YYWW (6) | SPDWFFNYYWW (23) |
| 7 | GNWHGTSPDWFFN | MTEQSEQTPAEYIPPMLVEVGEFTEDTLGNWHGT |
| | YYA (7) | SPDWFFNYYA (24) |
| 8 | GNWHGTSPDWFFN | MTEQSEQTPAEYIPPMLVEVGEFTEDTLGNWHGT |
| | YYF (8) | SPDWFFNYYF (25) |
| 9 | GNWHGTSPDWFFN | MTEQSEQTPAEYIPPMLVEVGEFTEDTLGNWHGT |
| | YYY (9) | SPDWFFNYYY (26) |
| 10 | GNWHGTSPDWFFN | MTEQSEQTPAEYIPPMLVEVGEFTEDTLGNWHGT |
| | YYNYYW (10) | SPDWFFNYYNYYW (27) |
| 11 | GNWHGTSPDWFFN | MTEQSEQTPAEYIPPMLVEVGEFTEDTLGNWHGT |
| | YYNIIW (11) | SPDWFFNYYNIIW (28) |
| 12 | GNWHGTSPDWFFN | MTEQSEQTPAEYIPPMLVEVGEFTEDTLGNWHGT |
| | YYAHLDIIW (12) | SPDWFFNYYAHLDIIW (29) |
| 13 | GNWHGTSPDWFFN | MTEQSEQTPAEYIPPMLVEVGEFTEDTLGNWHGT |
| | YYTrn (13) | SPDWFFNYYTrn (30) |
| 14 | GNWHGTSPDWFFN | MTEQSEQTPAEYIPPMLVEVGEFTEDTLGNWHGT |
| | YYW—OMe (14) | SPDWFFNYYW—OMe (31) |
| 15 | GNWHGTSPDWFFN | MTEQSEQTPAEYIPPMLVEVGEFTEDTLGNWHGT |
| | YYW—OBn (15) | SPDWFFNYYW—OBn (32) |
| 16 | GNWHGTSPDWFFN | MTEQSEQTPAEYIPPMLVEVGEFTEDTLGNWHGT |
| | YYW—NH2 (16) | SPDWFFNYYW—NH₂ (33) |
| 17 | GNWHGTSPDWFFN | MTEQSEQTPAEYIPPMLVEVGEFTEDTLGNWHGT |
| | YYNal (17) | SPDWFFNYYNal (34) |
| 42 | GNWHGTAPDWFFN | MTEQSEQTPAEYIPPMLVEVGEFTEDTLGNWHGT |
| | YY (42) | APDWFFNYY (57) |
| 43 | GNWHGTAPDWFFN | MTEQSEQTPAEYIPPMLVEVGEFTEDTLGNWHGT |
| | YYWW (43) | APDWFFNYYWW (58) |
| 44 | GNWHGTAPDWFFN | MTEQSEQTPAEYIPPMLVEVGEFTEDTLGNWHGT |
| | YYA (44) | APDWFFNYYA (59) |
| 45 | GNWHGTAPDWFFN | MTEQSEQTPAEYIPPMLVEVGEFTEDTLGNWHGT |
| | YYF (45) | APDWFFNYYF (60) |
| 46 | GNWHGTAPDWFFN | MTEQSEQTPAEYIPPMLVEVGEFTEDTLGNWHGT |
| | YYY (46) | APDWFFNYYY (61) |
| 47 | GNWHGTAPDWFFN | MTEQSEQTPAEYIPPMLVEVGEFTEDTLGNWHGT |
| | YYNYYW (47) | APDWFFNYYNYYW (62) |
| 48 | GNWHGTAPDWFFN | MTEQSEQTPAEYIPPMLVEVGEFTEDTLGNWHGT |
| | YYNIIW (48) | APDWFFNYYNIIW (63) |
| 49 | GNWHGTAPDWFFN | MTEQSEQTPAEYIPPMLVEVGEFTEDTLGNWHGT |
| | YYAHLDIIW (49) | APDWFFNYYAHLDI1W (64) |
| 50 | GNWHGTAPDWFFN | MTEQSEQTPAEYIPPMLVEVGEFTEDTLGNWHGT |
| | YYTrn (50) | APDWFFNYYTrn (65) |
| 51 | GNWHGTAPDWFFN | MTEQSEQTPAEYIPPMLVEVGEFTEDTLGNWHGT |
| | YYW—OMe (51) | APDWFFNYYW—OMe (66) |
| 52 | GNWHGTAPDWFFN | MTEQSEQTPAEYIPPMLVEVGEFTEDTLGNWHGT |
| | YYW—OBn (52) | APDWFFNYYW—OBn (67) |
| 53 | GNWHGTAPDWFFN | MTEQSEQTPAEYIPPMLVEVGEFTEDTLGNWHGT |
| | YYW—NH2 (53) | APDWFFNYYW—NH2 (68) |
| 54 | GNWHGTAPDWFFN | MTEQSEQTPAEYIPPMLVEVGEFTEDTLGNWHGT |
| | YYNal (54) | APDWFFNYYNal (69) |
| 55 | GNWHGTAPDWFFN | MTEQSEQTPAEYIPPMLVEVGEFTEDTLGNWHGT |
| | YYWIIW (55) | APDWFFNYYWIIW (70) |
| 56 | GNWHGTSPDWFFN | MTEQSEQTPAEYIPPMLVEVGEFTEDTLGNWHGT |
| | YYWIIW (56) | SPDWFFNYYWIIW (71) |

W—OMe: Tryptophan having a C-terminal methyl ester group (—CO₂Me) in place of the carboxylic acid group (—CO₂H);
W—OBn: Tryptophan having a C-terminal benzyl ester group (—CO₂Bn) in place of the carboxylic acid group (—CO₂H);
W—NH₂: Tryptophan having a C-terminal amide group (—CONH₂) in place of the carboxylic acid group (—CO₂H);
W-7-OH: 7-hydroxyl-trptophan;
Nal: 2-naphthylalanine; and
Trn: Aza derivative of Tryptophan: (2S)-2-amino-3-(1H-pyrrolo[5,4-b]pyridin-3-yl)propanoic acid having the structure of

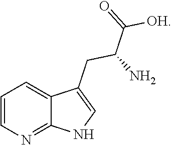 [SMILES: N[C@H](Cc1c[nH]c2ncccc12)C(=O)O]

In some embodiments, the lasso peptide comprises an amino acid sequence selected from SEQ ID NOS:1-17 and 42-56. In some embodiments, the lasso peptide consists essentially of an amino acid sequence selected from SEQ ID NOS:1-17 and 42-56. In some embodiments, the lasso peptide consists of an amino acid sequence selected from SEQ ID NOS:1-17 and 42-56.

In some embodiments, a lasso peptide comprises an amino acid sequence selected from SEQ ID NOS:1-17 and 42-56, and possesses the lariat conformation through G1-D9 cyclization. In some embodiments, the lasso peptide consists essentially of an amino acid sequence selected from SEQ ID NOS:1-17 and 42-56, and possesses the lariat conformation through G1-D9 cyclization. In some embodiments, the lasso peptide consists of an amino acid sequence selected from SEQ ID NOS:1-17 and 42-56, and possesses the lariat conformation through G1-D9 cyclization.

In some embodiments, the lasso peptides of the present disclosure compete with endothelin for binding with endothelin B receptor, where the endothelin is selected from endothelin-1, endothelin-2 or endothelin-3.

In some embodiments, the lasso peptides of the present disclosure preferentially bind to endothelin B receptor (ETBR) over endothelin A receptor (ETAR). In some embodiments, the lasso peptide preferentially binds to ETBR with a K_{D} that is about 75%, about 50%, about 25%, about 10%, about 5%, about 2.5%, or about 1% of the K_{D} exhibited relative to binding of ETAR. In some embodiments, the ratio between the K_{D} exhibited by the lasso peptide when binding to the ETAR and the K_{D} exhibited when binding to ETBR is at least 2 fold, at least 3 fold, at least 4 fold, at least 5 fold, at least 10 fold, at least 20 fold, at least 100 fold, at least 500 fold, at least 10³ fold, at least 10⁴ fold, or at least 10⁵ fold. In some embodiments, the lasso peptides of the present disclosure specifically bind to ETBR and do not exhibit detectable binding to ETAR.

In some embodiments, the lasso peptides of the present disclosure selectively inhibits or attenuates endothelin B receptor (ETBR) over endothelin A receptor (ETAR). In some embodiments, the lasso peptide selectively inhibits or attenuates ETBR with an IC50 less than half of the IC50 exhibited relative to ETAR. In some embodiments, the lasso peptide selectively inhibits or attenuates ETBR with an IC50 that is about 75%, about 50%, about 25%, about 10%, about 5%, about 2.5%, or about 1% of the IC50 exhibited relative to the ETAR. In some embodiments, the ratio between the IC50 exhibited by the lasso peptide with respect to the ETAR and the IC50 exhibited with respect to the ETBR is at least 2 fold, at least 3 fold, at least 4 fold, at least 5 fold, at least 10 fold, at least 20 fold, at least 100 fold, at least 500 fold, at least 10³ fold, at least 10⁴ fold, or at least 10⁵ fold. In some embodiments, the lasso peptides of the present disclosure specifically inhibits or attenuates ETBR and do not exhibit detectable inhibition or attenuation of ETAR.

In some embodiments, the lasso peptides of the present disclosure preferentially binds to endothelin B receptor-1 (ETBR1) over endothelin B receptor-2 (ETBR2). In some embodiments, the lasso peptide preferentially binds to ETBR1 with a K_{D} that is about 75%, about 50%, about 25%, about 10%, about 5%, about 2.5%, or about 1% of the K_{D} exhibited relative to binding of ETBR2. In some embodiments, the ratio between the K_{D} exhibited by the lasso peptide when binding to the ETBR2 and the K_{D} exhibited when binding to ETBR1 is at least 2 fold, at least 3 fold, at least 4 fold, at least 5 fold, at least 10 fold, at least 20 fold, at least 100 fold, at least 500 fold, at least 10³ fold, at least 10⁴ fold, or at least 10⁵ fold. In some embodiments, the lasso peptides of the present disclosure specifically bind to ETBR1 and do not exhibit detectable binding to ETAR2. In some embodiments, the lasso peptides of the present disclosure specifically binds to ETBR1 and do not exhibit detectable binding to ETBR2.

In some embodiments, the lasso peptides of the present disclosure selectively inhibits or attenuates endothelin B receptor-1 (ETBR1) over endothelin B receptor-2 (ETBR2). In some embodiments, the lasso peptide selectively inhibits or attenuates ETBR1 with an IC50 less than half of the IC50 exhibited relative to ETBR2. In some embodiments, the lasso peptide selectively inhibits or attenuates ETBR2 with an IC50 that is about 75%, about 50%, about 25%, about 10%, about 5%, about 2.5%, or about 1% of the IC50 exhibited relative to the ETBR1. In some embodiments, the ratio between the IC50 exhibited by the lasso peptide with respect to the ETBR2 and the IC50 exhibited with respect to the ETBR1 is at least 2 fold, at least 3 fold, at least 4 fold, at least 5 fold, at least 10 fold, at least 20 fold, at least 100 fold, at least 500 fold, at least 10³ fold, at least 10⁴ fold, or at least 10⁵ fold. In some embodiments, the lasso peptides of the present disclosure specifically inhibits or attenuates ETBR1 and do not exhibit detectable inhibition or attenuation of ETBR2.

In some embodiments, the lasso peptides of the present disclosure preferentially binds to endothelin B receptor-2 (ETBR2) over endothelin B receptor-1 (ETBR1). In some embodiments, the lasso peptide preferentially binds to ETBR2 with a K_{D} that is about 75%, about 50%, about 25%, about 10%, about 5%, about 2.5%, or about 1% of the K_{D} exhibited relative to binding of ETBR1. In some embodiments, the ratio between the K_{D} exhibited by the lasso peptide when binding to the ETBR1 and the K_{D} exhibited when binding to ETBR2 is at least 2 fold, at least 3 fold, at least 4 fold, at least 5 fold, at least 10 fold, at least 20 fold, at least 100 fold, at least 500 fold, at least 10³ fold, at least 10⁴ fold, or at least 10⁵ fold. In some embodiments, the lasso peptides of the present disclosure specifically bind to ETBR2 and do not exhibit detectable binding to ETAR1. In some embodiments, the lasso peptides of the present disclosure specifically binds to ETBR2 and do not exhibit detectable binding to ETBR1.

In some embodiments, the lasso peptides of the present disclosure selectively inhibits or attenuates endothelin B receptor-2 (ETBR2) over endothelin B receptor-1 (ETBR1). In some embodiments, the lasso peptide selectively inhibits or attenuates ETBR2 with an IC50 less than half of the IC50 exhibited relative to ETBR1. In some embodiments, the lasso peptide selectively inhibits or attenuates ETBR1 with an IC50 that is about 75%, about 50%, about 25%, about 10%, about 5%, about 2.5%, or about 1% of the IC50 exhibited relative to the ETBR2. In some embodiments, the ratio between the IC50 exhibited by the lasso peptide with respect to the ETBR1 and the IC50 exhibited with respect to the ETBR2 is at least 2 fold, at least 3 fold, at least 4 fold, at least 5 fold, at least 10 fold, at least 20 fold, at least 100 fold, at least 500 fold, at least 10³ fold, at least 10⁴ fold, or at least 10⁵ fold. In some embodiments, the lasso peptides of the present disclosure specifically inhibits or attenuates ETBR2 and do not exhibit detectable inhibition or attenuation of ETBR1.

In some embodiments, the lasso peptides of the present disclosure, upon binding to an ETBR, inhibits the ETBR. In some embodiments, the lasso peptides of the present disclosure inhibits at least one ETBR-mediated signaling pathways. In some embodiments, the lasso peptides of the present disclosure, downregulates ETBR expression on the surface of neoplastic cells produced by the proliferative disease. In some embodiments, the lasso peptides of the present disclosure downregulates ETBR expression on endothelin cells in the microenvironment of the neoplastic cells produced by the proliferative disease. In some embodiments, the lasso peptides of the present disclosure, upon binding to ETBR, downregulates ETBR expression by at least 1%, 2%, 5%, 10%, 20%, 25%, 50%, 75%, 90%, 95%, 99% or 100%.

In some embodiments, the lasso peptides of the present disclosure, upon binding to ETBR, inhibits at least one ETBR-mediated signaling pathways. In particular embodiments, the inhibition of ETBR-mediated signaling pathway is measured by (i) inhibition of release of relaxing factors; (ii) upregulation of intercellular adhesion molecule-1 (ICAM-1) expression and clustering; (iii) increasing in migration of intraepithelial tumor infiltrating leukocytes (TILs) into the microenvironment of the neoplastic cells; (iv) inhibition of angiogenesis in the microenvironment of neoplastic cells; (v) inhibition on growth and/or metastasis of neoplastic cells; (vi) increasing in apoptosis of neoplastic cells; or any combination of (i) to (vi). In particular embodiments, the relaxing factors are selected from nitric oxide, prostacyclin, and endothelium-derived hyperpolarizing factor, Ca2+, protein kinase C, mitogen-activated protein kinase, or any combination thereof. In specific embodiments, the TILs comprises neutrophils, T cells, B cells, NK cells, monocytes or a combination thereof. In specific embodiments, the monocytes comprise macrophages and/or dendritic cells. In some embodiments, the any of the above activities (i) to (vi) is inhibited at least 1%, 2%, 5%, 10%, 20%, 25%, 50%, 75%, 90%, 95%, 99% or 100%.

In some embodiments, the inhibition of at least one ETBR-mediated signaling pathway occurs simultaneously as the downregulation of ETBR expression. In some embodiments, the inhibition of at least one ETBR-mediated signaling pathway occurs before the downregulation of ETBR expression. In some embodiments, the inhibition of at least one ETBR-mediated signaling pathway occurs after the downregulation of ETBR expression. In some embodiments, downregulation of ETBR expression occurs about 1 hour after the inhibition of the at least one ETBR-mediated signaling pathway. In some embodiments, downregulation of ETBR expression occurs about 2 hour after the inhibition of the at least one ETBR-mediated signaling pathway. In some embodiments, downregulation of ETBR expression occurs about 3 hour after the inhibition of the at least one ETBR-mediated signaling pathway. In some embodiments, downregulation of ETBR expression occurs about 4 hour after the inhibition of the at least one ETBR-mediated signaling pathway. In some embodiments, downregulation of ETBR expression occurs about 5 hour after the inhibition of the at least one ETBR-mediated signaling pathway. In some embodiments, downregulation of ETBR expression occurs about 10 hour after the inhibition of the at least one ETBR-mediated signaling pathway.

5.4.2 Biosynthesis of Endothelin Receptor Antagonist Lasso Peptides

In a related aspect of the present disclosure, provided herein are methods and systems for producing lasso peptides. In certain embodiments, the methods provided herein can produce a large quantities of matured, functional lasso peptides in a short period of time. In some embodiments, the methods provided herein can produce a plurality of diversified species of lasso peptides and/or related molecules thereof simultaneously.

5.4.2.1 Genomic Mining Tools for Genes Coding Natural Lasso Peptides

Some naturally existing lasso peptides are encoded by a lasso peptide biosynthetic gene cluster, which typically comprises three main genes: one encodes for a lasso precursor peptide (referred to as Gene A), and two encode for processing enzymes including a lasso peptidase (referred to as Gene B) and a lasso cyclase (referred to as Gene C). The lasso precursor peptide comprises a lasso core peptide and additional peptidic fragments known as the “leader sequence” that facilitates recognition and processing by the processing enzymes. The leader sequence may determine substrate specificity of the processing enzymes. The processing enzymes encoded by the lasso peptide gene cluster convert the lasso precursor peptide into a matured lasso peptide having the lariat-like topology. Particularly, the lasso peptidase removes additional sequences from the precursor peptide to generate a lasso core peptide, and the lasso cyclase cyclizes a terminal portion of the core peptide around a terminal tail portion to form the lariat-like topology. Some lasso gene clusters further encodes for additional protein elements that facilitates the post-translational modification, including a facilitator protein known as the post-translationally modified peptide (RiPP) recognition element (RRE). Some lasso gene clusters further encodes for lasso peptide transporters, kinases, or proteins that play a role in immunity, such as isopeptidase. (Burkhart, B. J., et al., Nat. Chem. Biol., 2015, 11, 564-570; Knappe, T. A. et al., J. Am. Chem. Soc., 2008, 130, 11446-11454; Solbiati, J. O. et al. J. Bacteriol., 1999, 181, 2659-2662; Fage, C. D., et al., Angew. Chem. Int. Ed., 2016, 55, 12717-12721; Zhu, S., et al., J. Biol. Chem. 2016, 291, 13662-13678).

Computer-based genome-mining tools can be used to identify lasso biosynthetic gene clusters based on known genomic information. For example, one algorithm known as RODEO can rapidly analyze a large number of biosynthetic gene clusters (BGCs) by predicting the function for genes flanking query proteins. This is accomplished by retrieving sequences from GenBank followed by analysis with HMMER3. The results are compared against the Pfam database with the data being returned to the users in the form of spreadsheet. For analysis of BGCs not encoding proteins not covered by Pfam, RODEO allows usage of additional pHMMs (either curated databases or user-generated). Taking advantage of RODEO's ability to rapidly analyze genes neighboring a query, it is possible to compile a list of all observable lasso peptide biosynthetic gene clusters in GeneBank (Online Methods). A comprehensive evaluation of this data set would provide great insight into the lasso peptide family. Lasso peptide biosynthetic gene clusters can be identified by looking for the local presence of genes encoding proteins matching the Pfams for the lasso cyclase, lasso peptidase, and RRE.

To confidently predict lasso precursors, RODEO next performed a six-frame translation of the intergenic regions within each of the identified potential lasso biosynthetic gene clusters. The resulting peptides can be assessed based on length and essential sequence features and split into predicted leader and core regions. A series of heuristics based on known lasso peptide characteristics can be defined to predict precursors from a pool of false positives. After optimization of heuristic scoring, good prediction accuracy for biosynthetic gene clusters closely related to known lasso peptides can be obtained.

Machine learning, particularly, support vector machine (SVM) classification, would be effective in locating precursor peptides from predicted BGCs more distant to known lasso peptides. SVM is well-suited for RiPP discovery due to availability of SVM libraries that perform well with large data sets with numerous variables and the ability of SVM to minimize unimportant features. The SVM classifier can be optimized using a randomly selected and manually curated training set from the unrefined whole data. Of these, a random subpopulation was withheld as a test set to avoid over-fitting. By combining SVM classification with motif (MEME) analysis, along with our original heuristic scoring, prediction accuracy was greatly enhanced as evaluated by recall and precision metrics. This tripartite procedure can yield a high-scoring, well-separated population of lasso precursor peptide from candidate peptides. The training set was found to display nearly identical scoring distributions upon comparison to the full data set.

Other examples of genomic or biosynthetic gene search engine that can be used in connection with the present disclosure include the WARP DRIVE BIO™ software, anti-SMASH (ANTI-SMASH™) software (See: Bun, K., et al, Nucleic Acids Res., 2017, 45, W36-W41), iSNAP™ algorithm (See: Ibrahim, A., et al., Proc. Nat. Acad. Sci., USA., 2012, 109, 19196-19201), CLUSTSCAN™ (Starcevic, et al., Nucleic Acids Res., 2008, 36, 6882-6892), NP searcher (Li et al. (2009) Automated genome mining for natural products. BMC Bioinformatics, 10, 185), SBSPKS™ (Anand, et al. Nucleic Acids Res., 2010, 38, W487-W496), BAGEL3™ (Van Heel, et al., Nucleic Acids Res., 2013, 41, W448-W453), SMURF™ (Khaldi et al., Fungal Genet. Biol., 2010, 47, 736-741), ClusterFinder (CLUSTERFINDER™) or ClusterBlast (CLUSTERBLAST™) algorithms, and an Integrated Microbial Genomes (IMG)-ABC system (DOE Joint Genome Institute (JGI)). In some embodiments, lasso peptide biosynthetic gene clusters for use in CFB methods and processes as provided herein are identified by mining genome sequences of known bacterial natural product producers using established genome mining tools, such as anti-SMASH, BAGEL3, and RODEO. These genome mining tools can also be used to identify novel biosynthetic genes (e.g., for use in CFB systems and processes as provided herein) within metagenomic based DNA sequences. Lasso peptide biosynthetic gene clusters can be used in the methods and systems described herein to produce various lasso peptides.

5.4.2.2 Cell-Free Biosynthesis of Lasso Peptides

In one aspect, provided herein are methods for producing lasso peptides comprising sequences selected from SEQ ID NOS:1-17 and 42-56 in vitro cell-free biosynthesis (CFB) methods. CFB methods employ the enzymes and the biosynthetic and metabolic machinery present inside cells, but without using living cells. CFB methods allow rapid expression of natural biosynthetic genes and pathways and facilitate targeted or phenotypic activity screening of natural products, without the need for plasmid-based cloning or in vivo cellular propagation, thus enabling rapid process/product pipelines (e.g., creation of large quantity of lasso peptide in a short time). Features of the CFB methods for lasso peptide production include that oligonucleotides (linear or circular constructs of DNA or RNA) encoding a minimal set of lasso peptide biosynthesis pathway genes (e.g., Genes A-C in a lasso peptide biosynthetic gene cluster) may be added to a cell extract containing in vitro TX-TL machinery for transcribing and translating the genes into the functional enzymes and lasso precursor peptides for production of lasso peptides. Accordingly, the CFB methods can produce in a CFB reaction mixture at least two or more of the lasso peptide variants.

In some embodiments, the method for producing a lasso peptide comprises (a) providing a CFB system comprising a minimal set of lasso peptide biosynthesis components; and (b) incubating the CFB system under a suitable condition to produce the lasso peptide.

In some embodiments, the minimal set of lasso peptide biosynthesis components comprises one or more components functions to provide a lasso precursor peptide, and one or more components function to process the lasso precursor peptide into the lasso peptide. In some embodiments, the one or more components function to process the lasso precursor peptide into the lasso peptide consist of a lasso peptidase and a lasso cyclase. In some embodiments, the one or more components function to process the lasso precursor peptide into the lasso peptide consists of a lasso peptidase, a lasso cyclase and an RRE.

In some embodiments, the minimal set of lasso peptide biosynthesis components comprises one or more components functions to provide a lasso core peptide, and one or more components function to process the lasso core peptide into the lasso peptide. In some embodiments, the one or more components function to process the lasso core peptide into the lasso peptide comprises one or more selected from a lasso peptidase, a lasso cyclase and an RRE. In some embodiments, the one or more components function to process the lasso core into the lasso peptide consist of a lasso cyclase.

In various embodiments, the one or more components function to provide a peptide or protein (e.g., a lasso precursor peptide, a lasso core peptide, or lasso peptide biosynthetic enzymes and proteins) in a CFB system can be provided in the form of the peptide or protein are provided in the form of the peptide or protein per se.

In some embodiments, at least some of the peptide or protein components in the CFB system can be natural peptides or polypeptides. In some embodiments, at least some of the peptide or protein components in the CFB system are derivatives of natural peptides or polypeptides. In some embodiments, at least some of the peptide or protein components in the CFB system are non-natural peptides. In some embodiments, the one or more peptide or protein components of the CFB system can be isolated from nature, such as isolated from microorganisms producing the lasso precursor peptides. In some embodiments, the one or more peptide or protein components of the CFB system can be synthetically or recombinantly produced, using methods known in the art. In some embodiments, the one or more peptide or protein components of the CFB system can be synthesized using the CFB system as described herein, followed by purifying the biosynthesized peptide or protein components from the CFB system.

In some embodiments, the CFB system comprises one or more fusion protein, or a polynucleotide encoding the fusion protein such that the CFB system is capable of producing the fusion protein through in vitro transcription and translation (TX-TL).

In some embodiments, the fusion protein comprised a lasso precursor peptide or a lasso core peptide fused to one or more lasso peptide biosynthesis components. In some embodiments, the one or more lasso peptide biosynthesis components are selected from (i) a lasso peptidase; (ii) a lasso cyclase; (iii) a RRE; or (iv) any combinations of (i) to (iii). In some embodiments, the one or more lasso peptide biosynthesis components are encoded by the same lasso peptide biosynthetic gene cluster. In other embodiments, the one or more lasso peptide biosynthesis components are encoded by different lasso peptide biosynthetic gene cluster.

In some embodiments, the fusion protein comprises an amino acid linker between the lasso peptidase or the lasso cyclase and the one or more additional peptide or polypeptide. In some embodiments, the fusion protein does not comprise an amino acid linker between the lasso peptidase or the lasso cyclase and the one or more additional peptide or polypeptide.

In specific embodiments, the fusion protein comprises a lasso precursor peptide fused to a lasso peptidase. In specific embodiments, the fusion protein comprises a lasso precursor peptide fused to a lasso cyclase. In specific embodiments, the fusion protein comprises a lasso precursor peptide fused to a RRE. In specific embodiments, the fusion protein comprises a lasso precursor peptide fused to a lasso peptidase and a lasso cyclase. In specific embodiments, the fusion protein comprises a lasso precursor peptide fused to a lasso peptidase and a RRE. In specific embodiments, the fusion protein comprises a lasso precursor peptide fused to a lasso cyclase and a RRE. In specific embodiments, the fusion protein comprises a lasso precursor peptide fused to a lasso peptidase, a lasso cyclase and RRE. In specific embodiments, the fusion protein comprises a lasso core peptide fused to a lasso peptidase. In specific embodiments, the fusion protein comprises a lasso core peptide fused to a lasso cyclase. In specific embodiments, the fusion protein comprises a lasso core peptide fused to a RRE. In specific embodiments, the fusion protein comprises a lasso core peptide fused to a lasso peptidase and a lasso cyclase. In specific embodiments, the fusion protein comprises a lasso core peptide fused to a lasso peptidase and a RRE. In specific embodiments, the fusion protein comprises a lasso core peptide fused to a lasso cyclase and a RRE. In specific embodiments, the fusion protein comprises a lasso core peptide fused to a lasso peptidase, a lasso cyclase and RRE.

In some embodiments, the fusion protein comprised a lasso precursor peptide or a lasso core peptide fused to one or more additional peptide or polypeptide. In some embodiments, the one or more additional peptide or polypeptide comprises a peptide or polypeptide that facilitates production of the lasso precursor peptide or lasso core peptide or the lasso peptide derived therefrom through cell-free biosynthesis. Examples of peptide or polypeptide that can be fused with a lasso precursor peptide or a lasso core peptide according to the present disclosure include but are not limited to (i) a peptide or polypeptide that increases the level of transcription of the lasso precursor peptide or lasso core peptide in the CFB system; (ii) a peptide or polypeptide that increases the level of translation of the lasso precursor peptide or lasso core peptide in the CFB system; (iii) a peptide or polypeptide that facilitates the processing of the lasso precursor peptide or lasso core peptide into the lasso peptide; (iv) a peptide or polypeptide that improves stability of the lasso precursor peptide or lasso core peptide or the lasso peptide derived therefrom; (v) a peptide or polypeptide that improves solubility of the lasso precursor peptide or lasso core peptide or the lasso peptide derived therefrom; (vi) a peptide or polypeptide that enables or facilitates the detection of the lasso precursor peptide or lasso core peptide or the lasso peptide derived therefrom; (vii) a peptide or polypeptide that enables or facilitates purification of the lasso precursor peptide or lasso core peptide or the lasso peptide derived therefrom; (viii) a peptide or polypeptide that enables or facilitates immobilization of the lasso precursor peptide or lasso core peptide or the lasso peptide derived therefrom; or (ix) any combination of (i) to (viii).

In some embodiments, the fusion protein comprised a lasso precursor peptide or a lasso core peptide fused to one or more additional peptide or polypeptide. In some embodiments, the one or more additional peptide or polypeptide comprises a biologically active peptide or polypeptide. Examples of biologically active peptide or polypeptide that can be fused with a lasso precursor peptide or lasso core peptide according to the present disclosure include but are not limited to (i) a peptide or polypeptide capable of binding to a target molecule (e.g., an antibody or an antigen); (ii) a peptide or polypeptide that enhance cell permeability of the fusion protein; (iii) a peptide or polypeptide capable of conjugating the fusion protein to at least one additional copy of the fusion protein; (iv) a peptide or polypeptide capable of linking the fusion protein to one or more peptidic or non-peptidic molecule; (v) a peptide or polypeptide capable of modulating activity of the lasso precursor peptide or lasso core peptide; (vi) a peptide or polypeptide capable of modulating activity of the lasso peptide derived from the lasso precursor peptide or the lasso core peptide; or (vii) any combinations of (i) to (vi).

In some embodiments, the fusion protein comprised a lasso peptidase or a lasso cyclase fused to one or more additional peptide or polypeptide. In some embodiments, the one or more additional peptide or polypeptide is fused to the N-terminus of the lasso peptidase or the lasso cyclase. In some embodiments, the one or more additional peptide or polypeptide is fused at the C-terminus of the lasso peptidase or the lasso cyclase. In some embodiments, a polynucleotide encoding the fusion protein comprises a nucleic acid sequence encoding the lasso peptidase or the lasso cyclase, wherein the 5′ end of the nucleic acid sequence is linked to a nucleic acid sequence encoding the one or more additional peptide or polypeptide. In some embodiments, a polynucleotide encoding the fusion protein comprises a nucleic acid sequence encoding the lasso peptidase or the lasso cyclase, wherein the 3′ end of the nucleic acid sequence is linked to a nucleic acid sequence encoding the one or more additional peptide or polypeptide. In some embodiments, the fusion protein comprises an amino acid linker between the lasso peptidase or the lasso cyclase and the one or more additional peptide or polypeptide. In some embodiments, the fusion protein does not comprise an amino acid linker between the lasso peptidase or the lasso cyclase and the one or more additional peptide or polypeptide.

In some embodiments, the fusion protein comprised a lasso peptidase or a lasso cyclase fused to one or more additional peptide or polypeptide. In some embodiments, the more additional peptide or polypeptide comprises a peptide or polypeptide encoded by a lasso peptide biosynthetic gene cluster. Examples of peptide or polypeptide that can be fused with a lasso precursor peptide or a lasso core peptide according to the present disclosure include but are not limited to (i) a lasso precursor peptide; (ii) a lasso core peptide; (iii) a lasso peptidase; (iv) a lasso cyclase, (v) a RRE; or (vi) any combinations of (i) to (vi). In specific embodiments, the fusion protein comprises at least one lasso cyclase and at least one lasso peptidase. In specific embodiments, the fusion protein comprises at least one lasso cyclase fused to a RRE. In specific embodiments, the fusion protein comprises at least one lasso peptidase fused to a RRE.

In some embodiments, the fusion protein comprised a lasso peptidase or a lasso cyclase fused to one or more additional peptide or polypeptide. In some embodiments, the one or more additional peptide or polypeptide comprises a peptide or polypeptide that facilitates production of the lasso peptidase or lasso cyclase through cell-free biosynthesis. Examples of peptide or polypeptide that can be fused with the lasso peptidase or lasso cyclase according to the present disclosure include but are not limited to (i) a peptide or polypeptide that increases the level of transcription of the lasso peptidase or lasso cyclase in the CFB system; (ii) a peptide or polypeptide that increases the level of translation of the lasso peptidase or lasso cyclase in the CFB system; (iii) a peptide or polypeptide that improves stability of the lasso peptidase or lasso cyclase; (vi) a peptide or polypeptide that improves solubility of the lasso peptidase or lasso cyclase; (v) a peptide or polypeptide that enables or facilitates the detection of the lasso peptidase or lasso cyclase; (vi) a peptide or polypeptide that enables or facilitates purification of the lasso peptidase or lasso cyclase; (vii) a peptide or polypeptide that enables or facilitates immobilization of the lasso peptidase or lasso cyclase; or (viii) any combination of (i) to (vii).

In some embodiments, the fusion protein comprised a lasso peptidase or a lasso cyclase fused to one or more additional peptide or polypeptide. In some embodiments, the one or more additional peptide or polypeptide comprises a biologically active peptide or polypeptide. Examples of biologically active peptide or polypeptide that can be fused with a lasso peptidase or a lasso cyclase according to the present disclosure include but are not limited to (i) a peptide or polypeptide capable of modulating the reaction catalyzing activity of the lasso peptidase or lasso cyclase; (ii) a peptide or polypeptide capable of modulating target specificity of the lasso peptidase or lasso cyclase; (iii) an enzyme having the same or different enzymatic activity as the lasso peptidase or lasso cyclase; or any combination of (i) to (iii).

In some embodiments, the fusion protein comprised a RIPP recognition element (RRE) fused to one or more additional peptide or polypeptide. In some embodiments, the one or more additional peptide or polypeptide is fused to the N-terminus of the RRE. In some embodiments, the one or more additional peptide or polypeptide is fused at the C-terminus of the RRE. In some embodiments, a polynucleotide encoding the fusion protein comprises a nucleic acid sequence encoding the RRE, wherein the 5′ end of the nucleic acid sequence is linked to a nucleic acid sequence encoding the one or more additional peptide or polypeptide. In some embodiments, a polynucleotide encoding the fusion protein comprises a nucleic acid sequence encoding the RRE, wherein the 3′ end of the nucleic acid sequence is linked to a nucleic acid sequence encoding the one or more additional peptide or polypeptide. In some embodiments, the fusion protein comprises an amino acid linker between the RRE and the one or more additional peptide or polypeptide. In some embodiments, the fusion protein does not comprise an amino acid linker between RRE and the one or more additional peptide or polypeptide.

In some embodiments, the fusion protein comprised a RIPP recognition element (RRE) fused to one or more additional peptide or polypeptide. In some embodiments, the more additional peptide or polypeptide comprises a peptide or polypeptide encoded by a lasso peptide biosynthetic gene cluster. Examples of peptide or polypeptide that can be fused with a lasso precursor peptide or a lasso core peptide according to the present disclosure include but are not limited to (i) a lasso precursor peptide; (ii) a lasso core peptide; (iii) a lasso peptidase; (iv) a lasso cyclase, (v) a RRE; or (vi) any combinations of (i) to (vi). In specific embodiments, the fusion protein comprises at least one lasso precursor peptide fused to a RRE. In specific embodiments, the fusion protein comprises at least one lasso core peptide fused to a RRE. In specific embodiments, the fusion protein comprises at least one lasso cyclase fused to a RRE. In specific embodiments, the fusion protein comprises at least one lasso peptidase fused to a RRE.

In some embodiments, the fusion protein comprised a RIPP recognition element (RRE) fused to one or more additional peptide or polypeptide. In some embodiments, the one or more additional peptide or polypeptide comprises a peptide or polypeptide that facilitates production of the RRE through cell-free biosynthesis. Examples of peptide or polypeptide that can be fused with the RRE according to the present disclosure include but are not limited to (i) a peptide or polypeptide that increases the level of transcription of the RRE in the CFB system; (ii) a peptide or polypeptide that increases the level of translation of the RRE in the CFB system; (iii) a peptide or polypeptide that improves stability of the RRE; (vi) a peptide or polypeptide that improves solubility of the RRE; (v) a peptide or polypeptide that enables or facilitates the detection of the RRE; (vi) a peptide or polypeptide that enables or facilitates purification of the RRE; (vii) a peptide or polypeptide that enables or facilitates immobilization of the RRE; or (viii) any combination of (i) to (vii).

In some embodiments, the fusion protein comprised a RIPP recognition element (RRE) fused to one or more additional peptide or polypeptide. In some embodiments, the one or more additional peptide or polypeptide comprises a biologically active peptide or polypeptide. Examples of biologically active peptide or polypeptide that can be fused with a RRE according to the present disclosure include but are not limited to (i) a peptide or polypeptide capable of modulating the reaction catalyzing activity of the lasso peptidase or lasso cyclase; (ii) a peptide or polypeptide capable of modulating target specificity of the lasso peptidase or lasso cyclase; (iii) an enzyme having the same or different enzymatic activity as the lasso peptidase or lasso cyclase; or any combination of (i) to (iii).

In particular embodiments, the lasso precursor peptide genes are fused at the 5′-terminus of the DNA template strand of the gene to oligonucleotide sequences that encode peptides or proteins, such as sequences encoding maltose-binding protein (MBP) or small ubiquitin-like modifier protein (SUMO), which enhance the stability, solubility, and production of the desired TX-TL products (Marblestone, J. G., et al., Protein Sci, 2006, 15, 182-189). In particular embodiments, the lasso precursor peptides are fused at the C-terminus of the leader sequences to form conjugates with peptides or proteins, such as maltose-binding protein or small ubiquitin-like modifier protein, which enhance the stability, solubility, and production of the fused MBP-lasso or SUMO-lasso precursor peptide.

In particular embodiments, the lasso precursor peptide genes or lasso core peptide genes are fused at the 3′-terminus of the DNA template strand of the gene to oligonucleotide sequences that encode peptides or proteins, such as sequences encoding maltose-binding protein (MBP) or small ubiquitin-like modifier protein (SUMO), which enhance the stability, solubility, and production of the desired TX-TL products. In particular embodiments, the lasso precursor peptides, lasso core peptides, or lasso peptides are fused at the N-terminus to form conjugates with peptides or proteins, such as maltose-binding protein or small ubiquitin-like modifier protein, which enhance the stability, solubility, and production of the fused MBP-lasso or SUMO-lasso precursor peptide.

In particular embodiments, the lasso precursor peptide genes or lasso core peptide genes are fused at the 5′-terminus of the DNA template strand of the gene to oligonucleotide sequences that encode peptides or proteins, with or without a linker, such as sequences encoding peptide tags for affinity purification or immobilization, including his-tags, strep-tags, or FLAG-tags. In some embodiments, the lasso precursor peptides, lasso core peptides, or lasso peptides are fused at the C-terminus of the core peptides to form conjugates with other peptides or proteins, with or without a linker, such as peptide tags for affinity purification or immobilization, including his-tags, strep-tags, or FLAG-tags.

In particular embodiments, lasso precursor peptides, lasso core peptides, or lasso peptides are fused to molecules that can enhance cell permeability or penetration into cells, for example through the use of arginine-rich cell-penetrating peptides such as TAT peptide, penetratin, and flock house virus (FHV) coat peptide (Brock, R., Bioconjug. Chem., 2014, 25, 863-868). In particular embodiments, a lasso precursor peptide gene or core peptide gene is fused at the 3′-terminus to oligonucleotide sequences that encode arginine-rich cell-penetrating peptides or proteins, including oligonucleotide sequences that encode penetratin, and flock house virus (FHV) coat peptide or similar peptides that contain guanidinium groups or a combination of lysine and guanidinium groups (Wender, P. A., et al., Adv. Drug Deliv. Rev., 2008, 60, 452-472). In particular embodiments, a lasso precursor peptide, lasso core peptide, or lasso peptide is fused at the C-terminus to peptides that promote cell penetration such as arginine-rich cell-penetrating peptides or proteins, including amino acid sequences that encode TAT peptide, penetratin, and flock house virus (FHV) coat peptide or similar peptides that contain guanidinium groups or a combination of lysine and guanidinium groups.

In particular embodiments, the lasso precursor peptide genes or lasso core peptide genes are fused at the 5′-terminus of the DNA template strand of the gene to oligonucleotide sequences that encode peptides or proteins, with or without a linker, such as sequences encoding peptide epitopes that are known to bind with high affinity to antibodies, cell surface proteins, or cell surface receptors, including cytokine binding epitopes, integrin ligand binding epitopes, and the like. In particular embodiments, the lasso precursor peptides, lasso core peptides, or lasso peptides are fused at the C-terminus to peptides or proteins, with or without a linker, such as peptide epitopes that are known to bind with high affinity to antibodies, cell surface proteins, or cell surface receptors, including cytokine binding epitopes, integrin ligand binding epitopes, and the like.

Additionally or alternatively, the one or more components function to provide a peptide or protein (e.g., a lasso precursor peptide, a lasso core peptide, or lasso peptide biosynthetic enzymes and proteins) in a CFB system can be provided in the form of a nucleic acid encoding the peptide or protein and in vitro TX-TL machinery capable of producing the peptide or protein vial in vitro TX-TL of the coding sequences. In various embodiments, the coding nucleic acid can be DNA, RNA or cDNA. In various embodiments, one or more coding nucleic acid sequences can be contained in the same nucleic acid molecule, such as a vector.

It is understood that when more than one coding nucleic acid sequences are included in a CFB system, such more than one encoding nucleic acid sequences can be introduced on separate nucleic acid molecules, on polycistronic nucleic acid molecules, or a combination thereof. For example, as disclosed herein, a microbial organism or a cell extract can be engineered to express two or more exogenous nucleic acids encoding lasso precursor peptide, lasso core peptide, lasso peptidase, lasso cyclase or RRE. In the case where two exogenous nucleic acids encoding a desired activity are introduced into a host microbial organism or into a cell extract, it is understood that the two exogenous nucleic acids can be introduced as a single nucleic acid, for example, on a single plasmid or as linear strands of DNA, or on separate plasmids, or can be integrated into the host chromosome at a single site or multiple sites, and still be considered as two exogenous nucleic acids. Similarly, it is understood that more than two exogenous nucleic acids can be introduced into a host organism or into a cell extract in any desired combination, for example, on a single plasmid, or on separate plasmids, or as linear strands of DNA, or can be integrated into the host chromosome at a single site or multiple sites.

In some embodiments, the in vitro TX-TL machinery is purified from a host cell. In some embodiments, the in vitro TX-TL machinery is provided in the form of a cell extract of a host cell. An exemplary procedure for obtaining a cell extract comprises the steps of (i) growing cells, (ii) breaking open or lysing the cells by mechanical, biological or chemical means, (iii) removing cell debris and insoluble materials e.g., by filtration or centrifugation, and (iv) optionally treating to remove residual RNA and DNA, but retaining the active enzymes and biosynthetic machinery for transcription and translation, and optionally the metabolic pathways for co-factor recycle, including but not limited to co-factors such as THF, S-adenosylmethionine, ATP, NADH, NAD and NADP and NADPH. In some embodiments, a cell extract may be further supplemented for improved performance in in vitro TX-TL.

In some embodiments, a cell extract can be further supplemented with some or all of the twenty proteinogenic naturally-occurring amino acids and corresponding transfer ribonucleic acids (tRNAs), and optionally, may be supplemented with additional components, including but not limited to: (1) glucose, xylose, fructose, sucrose, maltose, or starch, (2) adenosine triphosphate (ATP), and/or adenosine diphosphate (ADP), purine and guanidine nucleotides, adenosine triphosphate, guanosine triphosphate, cytosine triphosphate, and/or uridine triphosphate, or combinations thereof, (3) cyclic-adenosine monophosphate (cAMP) and/or 3-phosphoglyceric acid (3-PGA), (4) nicotinamide adenine dinucleotides NADH and/or NAD, or nicotinamide adenine dinucleotide phosphates, NADPH, and/or NADP, or combinations thereof, (5) amino acid salts such as magnesium glutamate and/or potassium glutamate, (6) buffering agents such as HEPES, TRIS, spermidine, or phosphate salts, (7) inorganic salts, including but not limited to, potassium phosphate, sodium chloride, magnesium phosphate, and magnesium sulfate, (8) cofactors such as folinic acid and co-enzyme A (CoA), L(−)-5-formyl-5,6,7,8-tetrahydrofolic acid (THF), and/or biotin, (8) RNA polymerase, (9) 1,4-dithiothreitol (DTT), (10) magnesium acetate, and/or ammonium acetate, and/or (11) crowding agents such as PEG 8000, Ficoll 70, or Ficoll 400, or combinations thereof. In some embodiments, the cell extracts or supplemented cell extracts can be used as a reaction mixture to carry out in vitro TX-TL. In some embodiments, supplementations or adjustments can be made to the cell extract to provide a suitable condition for lasso formation.

In some embodiments, the in vitro TX-TL machinery is provided in the form of a cell extract or supplemented cell extract of a host cell. In some embodiments, the host cell is the cell of the same organism where the coding nucleic acid is derived from. For CFB of lasso peptides and related molecules thereof, the coding nucleic acid sequences can be identified using one or more computer-based genomic mining tools described herein or known in the art. For example, U.S. Provisional Application Nos. 62/652,213 and 62/651,028 disclose thousands of sequences from lasso peptide biosynthetic gene clusters identified from various organisms, and provide GenBank accession numbers for various sequences for lasso precursor peptides, lasso peptidase, lasso cyclase and/or RRE. Host organisms where the lasso peptide biosynthetic gene clusters originate can be identified based on the GenBank accession numbers, including but not limited to Caulobacteraceae species (e.g., Caulobacter sp. K31, Caulobacter henricii), Streptomyces species (e.g. Streptomyces nodosus, Streptomyces caatingaensis), Burkholderiaceae species (e.g., Burkholderia thailandensis E264), Pseudomallei species, Bacillus species, Burkholderia species (e.g., Burkholderia thailandensis MSMB43, Burkholderia oklahomensis, Burkholderia pseudomallei), Sphingomonadaceae species (e.g., Sphingobium sp. YBL2, Sphingobium chlorophenolicum, Sphingobium yanoikuyae). In other embodiments, the host cell is a microbial organism known to be applicable to fermentation processes. Exemplary bacteria include species selected from Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Streptomyces albus, Clostridium acetobutylicum, Vibrio natriegens, Pseudomonas fluorescens, and Pseudomonas putida. Exemplary yeasts or fungi include species selected from Saccharomyces cerevisiae, Schizosaccharomyces pombe, Kluyveromyces lactis, Kluyveromyces marxianus, Aspergillus terreus, Aspergillus niger and Pichia pastoris. E. coli is a particularly useful host organism since it is a well characterized microbial organism suitable for genetic engineering. Other particularly useful host organisms include Vibrio natriegens, and yeast such as Saccharomyces cerevisiae.

In some embodiments, the CFB system is configured to produce a lasso peptide. In specific embodiments, the CFB system comprises one or more components configured to provide (i) a lasso precursor peptide, (ii) a lasso peptidase, (iii) a lasso cyclase. In specific embodiments, the CFB system comprises one or more components configured to provide (i) a lasso core peptide, and (ii) a lasso cyclase. In some embodiments, the CFB system further comprises one or more components configured to provide (iv) an RRE. In some embodiments, all of (i) to (iv) above are provided in the CFB system as the corresponding peptide or protein. In alternative embodiments, at least one of (i) to (iv) above is provided in the CFB system as a nucleic acid encoding the corresponding protein, and the CFB system further comprises in vitro TX-TL machinery for producing the corresponding protein from the coding nucleic acid. In these embodiments, the CFB systems can be incubated under a condition suitable for lasso formation to produce the lasso peptide. The incubation condition can be designed and adjusted based on various factors known to skilled artisan in the art, including for example, condition suitable for maintain stability of components of the CFB system, conditions suitable for the lasso processing enzymes to exert enzymatic activities, and/or conditions suitable for the in vitro TX-TL of the coding sequences present in the CFB system. Exemplary suitable conditions are illustrated in Examples 1 and 3 of the present disclosure.

Without being bound by the theory, it is contemplated that different lasso peptidase can process the same lasso precursor peptide into different lasso core peptide by recognizing and cleaving different leader peptide off the lasso precursor. Additionally, different lasso cyclase can process the same lasso core peptide into distinct lasso peptides by cyclizing the core peptide at different ring-forming amino acid residues. Additionally, different RREs can facilitate different processing by the lasso peptidase and/or lasso cyclase, and thus lead to formation of distinct lasso peptides from the same lasso precursor peptide.

Accordingly, in some embodiments, to produce a natural lasso peptide, the CFB system comprises the lasso precursor peptide, lasso peptidase, and lasso cyclase produced from coding sequences of the same lasso peptide biosynthetic gene cluster (such as Genes A, B, and C of the same lasso peptide biosynthetic gene cluster). In some embodiments, to produce a natural lasso peptide, the CFB system comprises the lasso precursor peptide, lasso peptidase, lasso cyclase, and RRE produced from coding sequences of the same lasso peptide biosynthetic gene cluster.

In some embodiments, to produce a natural lasso peptide, the CFB system comprises the lasso core peptide, and lasso cyclase produced from coding sequences of the same lasso peptide biosynthetic gene cluster (such as Genes A and C of the same lasso peptide biosynthetic gene cluster). In some embodiments, to produce a natural lasso peptide, the CFB system comprises the lasso core peptide, lasso cyclase, and RRE produced from coding sequences of the same lasso peptide biosynthetic gene cluster.

In alternative embodiments, to produce a derivative of a natural lasso peptide, at least two of the lasso precursor peptide, lasso peptidase and lasso cyclase in the CFB system are produced from coding sequences of different lasso peptide biosynthetic gene clusters (such as Gene A from one, and Genes B and C from another, lasso peptide biosynthetic gene cluster). In alternative embodiments, to produce a derivative of a natural lasso peptide, at least two of the lasso precursor peptide, lasso peptidase, lasso cyclase and RRE in the CFB system are produced from coding sequences of different lasso peptide biosynthetic gene clusters.

In alternative embodiments, to produce a derivative of a natural lasso peptide, the lasso core peptide and lasso cyclase in the CFB system are produced from coding sequences of different lasso peptide biosynthetic gene clusters (such as Gene A from one, and Gene C from another, lasso peptide biosynthetic gene cluster). In alternative embodiments, to produce a derivative of a natural lasso peptide, at least two of the lasso core peptide, lasso cyclase and RRE in the CFB system are produced from coding sequences of different lasso peptide biosynthetic gene clusters.

In some embodiments, to produce a derivative of a natural lasso peptide, a lasso precursor peptide is modified at the core peptide sequence, while the leader sequence is maintained the same. The modified precursor peptide can then processed by corresponding lasso peptidase and/or lasso cyclase into a matured lasso peptide with modified amino acid sequence.

For example, in specific embodiments, variants of a lasso precursor peptide having SEQ ID NOS:18-34 and 57-71 share the same leader sequence (as shown by the underlined portion in Table 1. This leader sequence is recognized by lasso peptide biosynthesis component proteins having SEQ ID NOS:35, 36 and/or 27, and hence precursor peptides of SEQ ID NOS:18-34 and 57-71 can be processed by the same lasso peptide biosynthesis component proteins into matured lasso peptides having different amino acid sequences selected from SEQ ID NOS:1-17 and 42-56.

Accordingly, in specific embodiments, the present method of cell-free biosynthesis of a lasso peptide comprises (a) contacting a peptide comprising a sequence selected from SEQ ID NOS:1-34 and 42-71 with a lasso peptide biosynthesis component in a cell-free biosynthesis reaction mixture; and (b) incubating the cell-free biosynthesis reaction mixture under a condition suitable for lasso formation to produce the lasso peptide. In some embodiments, the present method of cell-free biosynthesis of a lasso peptide comprises (a) contacting a peptide comprising a sequence selected from SEQ ID NOS:1-17 and 42-56 with a lasso peptide biosynthesis component comprising a lasso cyclase in a cell-free biosynthesis reaction mixture; and (b) incubating the cell-free biosynthesis reaction mixture under a condition suitable for lasso formation to produce the lasso peptide. In some embodiments, the lasso peptide biosynthesis component further comprises a lasso peptidase and/or a RRE. In some embodiments, the present method of cell-free biosynthesis of a lasso peptide comprises (a) contacting a peptide comprising a sequence selected from SEQ ID NOS:18-34 and 57-71 with a lasso peptide biosynthesis component comprising a lasso peptidase and lasso cyclase in a cell-free biosynthesis reaction mixture; and (b) incubating the cell-free biosynthesis reaction mixture under a condition suitable for lasso formation to produce the lasso peptide. In some embodiments, the lasso peptide biosynthesis component further comprises a RRE.

In various embodiments, the contacting step (a) comprises adding a first nucleic acid sequence encoding the peptide into the cell-free biosynthesis reaction mixture, and where the cell-free biosynthesis reaction mixture comprises in vitro TX-TL machinery and is configured to express the peptide. In some embodiments, the contacting step (a) comprises adding a second nucleic acid sequence encoding the lasso peptide biosynthesis component to the cell-free biosynthesis reaction mixture, and where the cell-free biosynthesis reaction mixture comprises in vitro TX-TL machinery configured to express the lasso peptide biosynthesis component. In some embodiments, the lasso peptide biosynthesis component comprises a lasso peptidase. In some embodiments, the lasso peptide biosynthesis component comprises a lasso cyclase. In some embodiments, the lasso peptide biosynthesis component further comprises a post-translationally modified peptide (RiPP) recognition element (RRE). Particularly, in some embodiments, the lasso peptidase comprises a sequence of SEQ ID NO:35. In some embodiments, the lasso cyclase comprises a sequence of SEQ ID NO:36. In some embodiments, the lasso cyclase comprises a sequence of SEQ ID NO:37.

More particularly, in some of those embodiments where the lasso peptide biosynthesis component comprises a lasso peptidase and a lasso cyclase, the contacting step (a) comprises adding the second nucleic acid sequence encoding the lasso cyclase and a third nucleic acid sequence encoding the lasso peptidase. In some of those embodiments where the lasso peptide biosynthesis component comprises a lasso cyclase and a post-translationally modified peptide (RiPP) recognition element (RRE), the contacting step (a) comprises adding the second nucleic acid sequence encoding the lasso cyclase and a fourth nucleic acid sequence encoding the RRE. In some of those embodiments where the lasso peptide biosynthesis component comprises a lasso peptidase, a lasso cyclase and a post-translationally modified peptide (RiPP) recognition element (RRE), and where the contacting step (a) comprises adding the second nucleic acid sequence encoding the lasso cyclase, a third nucleic acid sequence encoding the lasso peptidase and a fourth nucleic acid sequence encoding the RRE. Particularly, in some embodiments, the lasso peptidase comprises a sequence of SEQ ID NO:35. In some embodiments, the lasso cyclase comprises a sequence of SEQ ID NO:36. In some embodiments, the lasso cyclase comprises a sequence of SEQ ID NO:37. In some embodiments, at least two of the first, second, third and fourth nucleic acid sequences are in a same nucleic acid molecule. In some embodiments, the lasso peptide comprises an amino acid sequence selected from SEQ ID NOS:1-17 and 42-56 is G1-D9 cyclized. In some embodiments, the cell-free biosynthesis reaction mixture comprises cell extract or supplemented cell extract.

Additional lasso peptide biosynthesis components and corresponding leader sequences are known in the art, such as those disclosed in PCT application publication numbers: WO2019/191571, which is incorporated herein by reference in its entirety. Therefore, in some embodiments, to produce lasso peptides having amino acid sequences selected from NOS:1-17 and 42-56, the core peptide sequences (SEQ ID NOS:1-17 and 42-56) can be fused to any known leader sequence, thereby producing a lasso precursor peptide, and the method then employs one or more lasso peptide biosynthesis component capable of recognizing such leader sequence and processing the lasso precursor peptide into matured lasso peptides having SEQ II) NOS:1-17 and 42-56.

Accordingly, in some embodiments, provided herein is a method of cell-free biosynthesis of a lasso peptide having an amino acid sequence selected from SEQ ID NOS:1-17 and 42-56, wherein the method comprises (a) contacting a lasso precursor peptide comprising a leader sequence and a lasso core peptide sequence selected from SEQ ID NOS:1-17 and 42-56 with a lasso peptide biosynthesis component in a cell-free biosynthesis reaction mixture; and (b) incubating the cell-free biosynthesis reaction mixture under a condition suitable for lasso formation to produce the lasso peptide; wherein the lasso peptide biosynthesis component comprises a lasso peptidase capable of catalyzing removal of the leader sequence. Particularly, in these embodiments, the corresponding leader sequence and lasso peptide biosynthesis components can be those disclosed in PCT application publication No.: WO2019/191571. In some embodiments, at least two of the first, second, third and fourth nucleic acid sequences are in a same nucleic acid molecule. In some embodiments, the lasso peptide comprises an amino acid sequence selected from SEQ ID NOS:1-17 and 42-56 is G1-D9 cyclized. In some embodiments, the cell-free biosynthesis reaction mixture comprises cell extract or supplemented cell extract.

In some embodiments, cell-free biosynthesis of lasso peptides is conducted with isolated peptide and enzyme components in standard buffered media, such as phosphate-buffered saline or tris-buffered saline, in each case containing salts, ATP, and co-factors required for lasso peptidase and lasso cyclase enzymatic activity. In some embodiments, cell-free biosynthesis of lasso peptides is conducted using genes that require transcription (TX) and translation (TL) to afford the lasso precursor peptide and/or lasso peptide biosynthetic enzymes in situ, and such in vitro biosynthesis processes are conducted in cell extracts derived from prokaryotic or eukaryotic cells (See: Gagoski, D., et al., Biotechnol. Bioeng. 2016; 113: 292-300; Culler, S. et al., PCT Appl. No. WO2017/031399).

In some embodiments, CFB reactions are conducted with a minimal set of lasso peptide biosynthesis components combined with genes that encode additional peptides, proteins or enzymes, including genes that encode RiPP recognition elements (RREs) or oligonucleotides that encode RREs that are fused to the 5′ or 3′ end of a lasso precursor peptide gene, a lasso core peptide gene, a lasso peptidase gene or a lasso cyclase gene. In other embodiments, CFB reactions are conducted with a minimal set of lasso peptide biosynthesis components, including lasso precursor peptides, lasso peptidases, or lasso cyclase that are fused to RREs at the N-terminus or C-terminus. In other embodiments, CFB reactions are conducted with a minimal set of lasso peptide biosynthesis components combined and contacted with additional isolated proteins or enzymes, including RiPP recognition elements (RREs).

In some embodiments, CFB reactions are conducted with a minimal set of lasso peptide biosynthesis components combined and contacted with genes that encode additional proteins or enzymes, including genes that encode lasso peptide modifying enzymes such as N-methyltransferases, O-methyltransferases, biotin ligases, glycosyltransferases, esterases, acylases, acyltransferases, aminotransferases, amidases, hydroxylases, dehydrogenases, halogenases, kinases, RiPP heterocyclases, RiPP cyclodehydratases, peptidylarginine deiminase, and prenyltransferases.

In some embodiments, CFB reactions are conducted with a minimal set of lasso peptide biosynthesis components combined and contacted with additional isolated proteins or enzymes, including lasso peptide modifying enzymes such as N-methyltransferases, O-methyltransferases, biotin ligases, glycosyltransferases, esterases, acylases, acyltransferases, aminotransferases, amidases, hydroxylases, dehydrogenases, halogenases, kinases, RiPP heterocyclases, RiPP cyclodehydratases, peptidylarginine deiminase, and prenyltransferases.

CFB methods and systems provided herein for the synthesis of lasso peptides and related molecules thereof from a minimal set of lasso peptide biosynthetic pathway components, are conducted in a CFB reaction mixture, comprising one or more cell extracts that are supplemented with all twenty proteinogenic naturally occurring amino acids and corresponding transfer ribonucleic acids (tRNAs). Cell extracts used in the CFB reaction mixture, provided herein for the synthesis of lasso peptides and related molecules thereof from a minimal set of lasso peptide biosynthetic pathway components also may be supplemented with additional components, including but not limited to, glucose, xylose, fructose, sucrose, maltose, starch, adenosine triphosphate (ATP), and/or adenosine diphosphate (ADP), purine and guanidine nucleotides, adenosine triphosphate, guanosine triphosphate, cytosine triphosphate, and uridine triphosphate, cyclic-adenosine monophosphate (cAMP) and/or 3-phosphoglyceric acid (3-PGA), nicotinamide adenine dinucleotides NADH and/or NAD, or nicotinamide adenine dinucleotide phosphates, NADPH, and/or NADP, or combinations thereof, amino acid salts such as magnesium glutamate and/or potassium glutamate, buffering agents such as HEPES, TRIS, spermidine, or phosphate salts, inorganic salts, including but not limited to, potassium phosphate, sodium chloride, magnesium phosphate, and magnesium sulfate, folinic acid and co-enzyme A (CoA), crowding agents such as PEG 8000, Ficoll 70, or Ficoll 400, L(−)-5-formyl-5,6,7,8-tetrahydrofolic acid, RNA polymerase, biotin, 1,4-dithiothreitol (DTT), magnesium acetate, ammonium acetate, or combinations thereof. For a general description of cell-free extract production and preparation, see: Krinsky, N., et al., PLoS ONE, 2016, 11(10): e0165137.

In alternative embodiments, the preparation CFB reaction mixtures and cell extracts employed for the CFB methods as provided herein, comprises characterization of the CFB reaction mixtures and cell extracts using proteomic approaches to assess and quantify the proteome available for the production of lasso peptides and related molecules thereof In alternative embodiments, ¹³C metabolic flux analysis (MFA) and/or metabolomics studies are conducted on CFB reaction mixtures and cell extracts to create a flux map and characterize the resulting metabolome of the CFB reaction mixture and cell extract or extracts.

In other embodiments, the CFB method is performed using: one or a combination of two or more cell extracts from various “chassis” organisms, such as E. coli, optionally mixed with one or a combination of two or more cell extracts derived from other species, e.g., a native lasso peptide-producing organism or relative. This can give the advantage of a robust transcription/translation machinery, combined with any unknown components of the native species that might be needed for proper protein folding or activity, or to supply precursors for the lasso peptide pathway. In alternative embodiments, if these factors are known they can be expressed in the chassis organism prior to making the cell extract or these factors can be isolated and purified and added directly to the CFB reaction mixture or cell extract.

In alternative embodiments, CFB methods and systems provided herein to produce lasso peptides and related molecules thereof from a minimal set of lasso peptide biosynthetic pathway components, including the use of cell extracts for in vitro TX-TL systems express lasso peptide biosynthetic gene clusters without the regulatory constraints of the cell. In alternative embodiments, some or all of the lasso peptide pathway biosynthetic genes are refactored to remove native transcriptional and translational regulation. In alternative embodiments, some or all of the lasso peptide pathway biosynthetic genes are refactored and constructed into operons on plasmids.

In alternative embodiments, CFB methods, systems and processes, including in vitro TX-TL systems, provided herein to produce lasso peptides and related molecules thereof from a minimal set of lasso peptide biosynthetic pathway components, are cell-free platforms that can use whole cell, cytoplasmic or nuclear extract from a single organism such as E. coli or Saccharomyces cerevisiae (S. cerevisiae) or from an organism of the Actinomyces genus, e.g., a Streptomyces. In alternative embodiments, CFB methods, systems and processes, including in vitro TX-TL systems, provided herein to produce lasso peptides and related molecules thereof from a minimal set of lasso peptide biosynthetic pathway components, are cell-free platforms that can use mixtures of whole cell, cytoplasmic, and/or nuclear extracts from the same or different organisms. In alternative embodiments, strain engineering approaches as well as modification of the growth conditions are used (on the organism from which at least one extract is derived) towards the creation of cell extracts as provided herein, to generate mixed cell extracts with varying proteomic and metabolic capabilities in the final CFB reaction mixture. In alternative embodiments, both approaches are used to tailor or design a final CFB reaction mixture for the purpose of synthesizing and characterizing lasso peptides, or for the creation of lasso peptide analogs through combinatorial biosynthesis approaches.

In alternative embodiments, cell extracts used in the CFB methods, provided herein to produce lasso peptides and related molecules thereof from a minimal set of lasso peptide biosynthetic pathway components, comprise whole cell, cytoplasmic or nuclear extracts from a bacterial cell or eukaryotic cell, including insect, plant, fungal, yeast, or mammalian cells. In alternative embodiments, cell extracts used in the CFB methods, provided herein to produce lasso peptides and related molecules thereof from a minimal set of lasso peptide biosynthetic pathway components, comprise whole cell, cytoplasmic or nuclear extracts from a bacterial cell or eukaryotic cell, including insect, plant, fungal, yeast, or mammalian cells, and are designed, produced and processed in a way to maximize efficacy and yield in the production of desired lasso peptides or related molecules thereof.

In an alternative embodiment, cell extracts used in the CFB methods, provided herein to produce lasso peptides and related molecules thereof from a minimal set of lasso peptide biosynthetic pathway components, derive from at least two different bacterial cells, two different fungal cells; two different yeast cells, two different insect cells, two different plant cells or two different mammalian cells, or combinations of cell extracts from different species and genera thereof. In alternative embodiments, cell extracts used in the CFB methods, provided herein to produce lasso peptides and related molecules thereof from a minimal set of lasso peptide biosynthetic pathway components, comprises an extract derived from: an Escherichia or a Escherichia coli (E. coli); a Streptomyces or an Actinobacteria; an Ascomycota, Basidiomycota, or a Saccharomycetales; a Penicillium or a Trichocomaceae; a Spodoptera, a Spodoptera frugiperda, a Trichoplusia or a Trichoplusia ni; a Poaceae, a Triticum, or a wheat germ; a rabbit reticulocyte or a HeLa cell.

In alternative embodiments, cell extracts used in the CFB methods, provided herein to produce lasso peptides and related molecules thereof from a minimal set of lasso peptide biosynthetic pathway components, comprises a cell extract from or comprises an extract derived from: any prokaryotic and eukaryotic organism including, but not limited to, bacteria, including Archaea, eubacteria, and eukaryotes, including yeast, plant, insect, animal, and mammal, including human cells. In alternative embodiments, at least one of the cell extracts used in the CFB methods provided herein comprises an extract from or comprises an extract derived from: Escherichia coli, Saccharomyces cerevisiae, Saccharomyces kluyveri, Candida boidinii, Clostridium kluyveri, Clostridium acetobutylicum, Clostridium beijerinckii, Clostridium saccharoperbutylacetonicum, Clostridium perfringens, Clostridium difficile, Clostridium botulinum, Clostridium tyrobutyricum, Clostridium tetanomorphum, Clostridium tetani, Clostridium propionicum, Clostridium aminobutyricum, Clostridium subterminale, Clostridium sticklandii, Ralstonia eutropha, Mycobacterium bovis, Mycobacterium tuberculosis, Porphyromonas gingivalis, Arabidopsis thaliana, Thermus thermophilus, Pseudomonas species, including Pseudomonas aeruginosa, Pseudomonas putida, Pseudomonas stutzeri, Pseudomonas fluorescens, Homo sapiens, Oryctolagus cuniculus, Rhodobacter spaeroides, Thermo-anaerobacter brockii, Metallosphaera sedula, Leuconostoc mesenteroides, Chloroflexus aurantiacus, Roseiflexus castenholzii, Erythrobacter, Simmondsia chinensis, Acinetobacter species, including Acinetobacter calcoaceticus and Acinetobacter baylyi, Porphyromonas gingivalis, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Rattus norvegicus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Sus scrofa, Caenorhabditis elegans, Corynebacterium glutamicum, Acidaminococcus fermentans, Lactococcus lactis, Lactobacillus plantarum, Streptococcus thermophilus, Enterobacter aerogenes, Candida, Aspergillus terreus, Pedicoccus pentosaceus, Zymomonas mobilus, Acetobacter pasteurians, Kluyveromyces lactis, Eubacterium barkeri, Bacteroides capillosus, Anaerotruncus colihominis, Natranaerobius thermophilusm, Campylobacter jejuni, Haemophilus influenzae, Serratia marcescens, Citrobacter amalonaticus, Myxococcus xanthus, Fusobacterium nuleatum, Penicillium chrysogenum, marine gamma proteobacterium, butyrate producing bacterium, Nocardia iowensis, Nocardia farcinica, Streptomyces griseus, Schizosaccharomyces pombe, Geobacillus thermoglucosidasius, Salmonella typhimurium, Vibrio cholera, Heliobacter pylori, Nicotiana tabacum, Oryza sativa, Haloferax mediterranei, Agrobacterium tumefaciens, Achromobacter denitrificans, Fusobacterium nucleatum, Streptomyces clavuligenus, Acinetobacter baumanii, Mus musculus, Lachancea kluyveri, Trichomonas vaginalis, Trypanosoma brucei, Pseudomonas stutzeri, Bradyrhizobium japonicum, Mesorhizobium loti, Bos taurus, Nicotiana glutinosa, Vibrio vulnificus, Vibrio natriegens, Selenomonas ruminantium, Vibrio parahaemolyticus, Archaeoglobus fulgidus, Haloarcula marismortui, Pyrobaculum aerophilum, Mycobacterium smegmatis MC2 155, Mycobacterium avium subsp. paratuberculosis K-10, Mycobacterium marinum M, Tsukamurella paurometabola DSM 20162, Cyanobium PCC7001, Dictyostelium discoideum AX4.

In alternative embodiments, at least one cell, cytoplasmic or nuclear extract used in the CFB methods, provided herein to produce lasso peptides and related molecules thereof from a minimal set of lasso peptide biosynthetic pathway components, comprises a cell extract from or comprises an extract derived from: Acinetobacter baumannii Naval-82, Acinetobacter sp. ADP1, Acinetobacter sp. strain M-1, Actinobacillus succinogenes 130Z, Allochromatium vinosum DSM 180, Amycolatopsis methanolica, Arabidopsis thaliana, Atopobium parvulum DSM 20469, Azotobacter vinelandii DJ, Bacillus alcalophilus ATCC 27647, Bacillus azotoformans LMG 9581, Bacillus coagulans 36D1, Bacillus megaterium, Bacillus methanolicus MGA3, Bacillus methanolicus PB1, Bacillus methanolicus PB-1, Bacillus selenitireducens MLS10, Bacillus smithii, Bacillus subtilis, Burkholderia cenocepacia, Burkholderia cepacia, Burkholderia multivorans, Burkholderia pyrrocinia, Burkholderia stabilis, Burkholderia thailandensis E264, Burkholderiales bacterium Joshi_001, Butyrate producing bacterium L2-50, Campylobacter jejuni, Candida albicans, Candida boidinii, Candida methylica, Carboxydothermus hydrogenoformans, Carboxydothermus hydrogenoformans Z-2901, Caulobacter sp. AP07, Chloroflexus aggregans DSM 9485, Chloroflexus aurantiacus J-10-fl, Citrobacter freundii, Citrobacter koseri ATCC BAA-895, Citrobacter youngae, Clostridium, Clostridium acetobutylicum, Clostridium acetobutylicum ATCC 824, Clostridium acidurici, Clostridium aminobutyricum, Clostridium asparagiforme DSM 15981, Clostridium beijerinckii, Clostridium beijerinckii NCIMB 8052, Clostridium bolteae ATCC BAA-613, Clostridium carboxidivorans P7, Clostridium cellulovorans 743B, Clostridium difficile, Clostridium hiranonis DSM 13275, Clostridium hylemonae DSM 15053, Clostridium kluyveri, Clostridium kluyveri DSM 555, Clostridium ljungdahli, Clostridium ljungdahlii DSM 13528, Clostridium methylpentosum DSM 5476, Clostridium pasteurianum, Clostridium pasteurianum DSM 525, Clostridium perfringens, Clostridium perfringens ATCC 13124, Clostridium perfringens str. 13, Clostridium phytofermentans ISDg, Clostridium saccharobutylicum, Clostridium saccharoperbutylacetonicum, Clostridium saccharoperbutylacetonicum N1-4, Clostridium tetani, Corynebacterium glutamicum ATCC 14067, Corynebacterium glutamicum R, Corynebacterium sp. U-96, Corynebacterium variabile, Cupriavidus necator N-1, Cyanobium PCC7001, Desulfatibacillum alkenivorans AK-01, Desulfitobacterium hafniense, Desulfitobacterium metallireducens DSM 15288, Desulfotomaculum reducens MI-1, Desulfovibrio africanus str. Walvis Bay, Desulfovibrio fructosovorans JJ, Desulfovibrio vulgaris str. Hildenborough, Desulfovibrio vulgaris str. Miyazaki F, Dictyostelium discoideum AX4, Escherichia coli, Escherichia coli K-12, Escherichia coli K-12 MG1655, Eubacterium hallii DSM 3353, Flavobacterium frigoris, Fusobacterium nucleatum subsp. polymorphum ATCC 10953, Geobacillus sp. Y4. 1MC1, Geobacillus themodenitrificans NG80-2, Geobacter bemidjiensis Bem, Geobacter sulfurreducens, Geobacter sulfurreducens PCA, Geobacillus stearothermophilus DSM 2334, Haemophilus influenzae, Helicobacter pylori, Homo sapiens, Hydrogenobacter thermophilus, Hydrogenobacter thermophilus TK-6, Hyphomicrobium denitrificans ATCC 51888, Hyphomicrobium zavarzinii, Klebsiella pneumoniae, Klebsiella pneumoniae subsp. pneumoniae MGH 78578, Lactobacillus brevis ATCC 367, Leuconostoc mesenteroides, Lysinibacillus fusiformis, Lysinibacillus sphaericus, Mesorhizobium loti MAFF303099, Metallosphaera sedula, Methanosarcina acetivorans, Methanosarcina acetivorans C2A, Methanosarcina barkeri, Methanosarcina mazei Tuc01, Methylobacter marinus, Methylobacterium extorquens, Methylobacterium extorquens AM1, Methylococcus capsulatas, Methylomonas aminofaciens, Moorella thermoacetica, Mycobacter sp. strain JC1 DSM 3803, Mycobacterium avium subsp. paratuberculosis K-10, Mycobacterium bovis BCG, Mycobacterium gastri, Mycobacterium marinum M, Mycobacterium smegmatis, Mycobacterium smegmatis MC2 155, Mycobacterium tuberculosis, Nitrosopumilus salaria BD31, Nitrososphaera gargensis Ga9.2, Nocardia farcinica IFM 10152, Nocardia iowensis (sp. NRRL 5646), Nostoc sp. PCC 7120, Ogataea angusta, Ogataea parapolymorpha DL-1 (Hansenula polymorpha DL-1), Paenibacillus peoriae KCTC 3763, Paracoccus denitrificans, Penicillium chrysogenum, Photobacterium profundum 3TCK, Phytofermentans ISDg, Pichia pastoris, Picrophilus torridus DSM9790, Porphyromonas gingivalis, Porphyromonas gingivalis W83, Pseudomonas aeruginosa PA01, Pseudomonas denitrificans, Pseudomonas knackmussii, Pseudomonas putida, Pseudomonas sp, Pseudomonas syringae pv. syringae B728a, Pyrobaculum islandicum DSM 4184, Pyrococcus abyssi, Pyrococcus furiosus, Pyrococcus horikoshii OT3, Ralstonia eutropha, Ralstonia eutropha H16, Rhodobacter capsulatus, Rhodobacter sphaeroides, Rhodobacter sphaeroides ATCC 17025, Rhodopseudomonas palustris, Rhodopseudomonas palustris CGA009, Rhodopseudomonas palustris DX-1, Rhodospirillum rubrum, Rhodospirillum rubrum ATCC 11170, Ruminococcus obeum ATCC 29174, Saccharomyces cerevisiae, Saccharomyces cerevisiae S288c, Salmonella enterica, Salmonella enterica subsp. enterica serovar Typhimurium str. LT2, Salmonella enterica typhimurium, Salmonella typhimurium, Schizosaccharomyces pombe, Sebaldella termitidis ATCC 33386, Shewanella oneidensis MR-1, Sinorhizobium meliloti 1021, Streptomyces coelicolor, Streptomyces griseus subsp. griseus NBRC 13350, Sulfolobus acidocalarius, Sulfolobus solfataricus P-2, Synechocystis str. PCC 6803, Syntrophobacter fumaroxidans, Thauera aromatica, Thermoanaerobacter sp. X514, Thermococcus kodakaraensis, Thermococcus litoralis, Thermoplasma acidophilum, Thermoproteus neutrophilus, Thermotoga maritima, Thiocapsa roseopersicina, Tolumonas auensis DSM 9187, Trichomonas vaginalis G3, Trypanosoma brucei, Tsukamurella paurometabola DSM 20162, Vibrio cholera, Vibrio harveyi ATCC BAA-1116, Vibrio natriegens, Xanthobacter autotrophicus Py2, Yersinia intermedia, or Zea mays.

In alternative embodiments, cell extracts used in the CFB methods and processes, provided herein for the synthesis of lasso peptides and related molecules thereof from a minimal set of lasso peptide biosynthetic pathway components, e.g., including at least one of the cell, cytoplasmic or nuclear extracts, have added to them, or further comprise, supplemental ingredients, compositions or compounds, reagents, ions, trace metals, salts, or elements, buffers and/or solutions. In alternative embodiments, the CFB method and system of the present disclosure, provided herein for the synthesis of lasso peptides and related molecules thereof from a minimal set of lasso peptide biosynthetic pathway components, use or fabricate environmental conditions to optimize the rate of formation or yield of a lasso peptide or related molecules thereof.

In alternative embodiments, CFB reaction mixtures and cell extracts used in the CFB methods and systems, provided herein for the synthesis of lasso peptides and related molecules thereof from a minimal set of lasso peptide biosynthetic pathway components, are supplemented with a carbon source and other essential nutrients. The CFB production system, including cell extracts used in the CFB methods and processes, provided herein for the synthesis of lasso peptides and related molecules thereof from a minimal set of lasso peptide biosynthetic pathway components, can include, for example, any carbohydrate source. Such sources of sugars or carbohydrate substrates include glucose, xylose, maltose, arabinose, galactose, mannose, maltodextrin, fructose, sucrose and starch.

In alternative embodiments, CFB methods and systems provided herein for the synthesis of lasso peptides and related molecules thereof from a minimal set of lasso peptide biosynthetic pathway components, are conducted in a CFB reaction mixture, comprising cell extracts that are supplemented with all twenty proteinogenic naturally occurring amino acids and corresponding transfer ribonucleic acids (tRNAs). In alternative embodiments, cell extracts used in the CFB reaction mixture, provided herein for the synthesis of lasso peptides and related molecules thereof from a minimal set of lasso peptide biosynthetic pathway components, are supplemented with adenosine triphosphate (ATP), and/or adenosine diphosphate (ADP). In alternative embodiments, cell extracts used in the CFB reaction mixture, provided herein for the synthesis of lasso peptides and related molecules thereof from a minimal set of lasso peptide biosynthetic pathway components, are supplemented with glucose, xylose, maltose, arabinose, galactose, mannose, maltodextrin, fructose, sucrose and/or starch. In alternative embodiments, cell extracts used in the CFB reaction mixture, provided herein for the synthesis of lasso peptides and related molecules thereof from a minimal set of lasso peptide biosynthetic pathway components, are supplemented with purine and guanidine nucleotides, adenosine triphosphate, guanosine triphosphate, cytosine triphosphate, and uridine triphosphate. In alternative embodiments, cell extracts used in the CFB reaction mixture, provided herein for the synthesis of lasso peptides and related molecules thereof from a minimal set of lasso peptide biosynthetic pathway components, are supplemented with cyclic-adenosine monophosphate (cAMP) and/or 3-phosphoglyceric acid (3-PGA). In alternative embodiments, cell extracts used in the CFB reaction mixture, provided herein for the synthesis of lasso peptides and related molecules thereof from a minimal set of lasso peptide biosynthetic pathway components, are supplemented with nicotinamide adenine dinucleotides NADH and/or NAD, or nicotinamide adenine dinucleotide phosphates, NADPH, and/or NADP, or combinations thereof. In alternative embodiments, cell extracts used in the CFB reaction mixture, provided herein for the synthesis of lasso peptides and related molecules thereof from a minimal set of lasso peptide biosynthetic pathway components, are supplemented with amino acid salts such as magnesium glutamate and/or potassium glutamate. In alternative embodiments, cell extracts used in the CFB reaction mixture, provided herein for the synthesis of lasso peptides and related molecules thereof from a minimal set of lasso peptide biosynthetic pathway components, are supplemented with buffering agents such as HEPES, TRIS, spermidine, or phosphate salts. In alternative embodiments, cell extracts used in the CFB reaction mixture, provided herein for the synthesis of lasso peptides and related molecules thereof from a minimal set of lasso peptide biosynthetic pathway components, are supplemented with salts, including but not limited to, potassium phosphate, sodium chloride, magnesium phosphate, and magnesium sulfate. In alternative embodiments, cell extracts used in the CFB reaction mixture, provided herein for the synthesis of lasso peptides and related molecules thereof from a minimal set of lasso peptide biosynthetic pathway components, are supplemented with folinic acid and co-enzyme A (CoA). In alternative embodiments, cell extracts used in the CFB reaction mixture, provided herein for the synthesis of lasso peptides and related molecules thereof from a minimal set of lasso peptide biosynthetic pathway components, are supplemented with crowding agents such as PEG 8000, Ficoll 70, or Ficoll 400, or combinations thereof. For a general description of cell-free extract production and preparation, see: Krinsky, N., et al., PLoS ONE, 2016, 11(10): e0165137.

5.4.2.3 Cell-Based Biosynthesis of Lasso Peptides

In a related aspect, provided herein are also methods for producing lasso peptides having a sequence selected from SEQ ID NOS:1-17 and 42-56 using a non-naturally occurring microbial organism. Certain cell-based production methods involve cultivating or fermenting a microbial organism that is a natural producer of a lasso peptide of interest. Alternative cell-based production methods involve cloning the genes encoding lasso peptide biosynthesis component into an appropriate vector or plasmid, introducing that vector or plasmid into a microorganism, and propagating or cultivating that organism with the necessary nutrients and under conditions for heterologous production of recombinant lasso peptides of interest (Zhang, Y., et al, Heterologous production of microbial ribosomally synthesized and post-translationally modified peptides, Front. Microbiol., 2018, doi: 10.3389/fmicb.2018.01801).

Depending on the lasso peptide biosynthetic pathway constituents of a selected host microbial organism, the non-naturally occurring microbial organisms of the invention will include at least one exogenously expressed lasso peptide pathway-encoding nucleic acid and up to all encoding nucleic acids for one or more lasso peptide biosynthetic pathways. For example, lasso peptide biosynthesis can be established in a host deficient in a pathway enzyme or protein through exogenous expression of the corresponding encoding nucleic acid. In a host deficient in all enzymes or proteins of a lasso peptide pathway, exogenous expression of all enzyme or proteins in the pathway can be included, although it is understood that all enzymes or proteins of a pathway can be expressed even if the host contains at least one of the pathway enzymes or proteins. For example, exogenous expression of all enzymes or proteins in a pathway for production of a lasso peptide can be included, such as a lasso peptide precursor, a lasso peptide peptidase, a lasso peptide cyclase, and/or a lasso peptide RiPP recognition element (RRE).

Given the teachings and guidance provided herein, those skilled in the art will understand that the number of encoding nucleic acids to introduce in an expressible form will, at least, parallel the lasso peptide pathway deficiencies of the selected host microbial organism. Therefore, a non-naturally occurring microbial organism of the invention can have one, two, three, four, five, six, seven, eight, nine or ten, up to all nucleic acids encoding the enzymes or proteins constituting a lasso peptide biosynthetic pathway disclosed herein. In some embodiments, the non-naturally occurring microbial organisms also can include other genetic modifications that facilitate or optimize lasso peptide biosynthesis or that confer other useful functions onto the host microbial organism. One such other functionality can include, for example, augmentation of the synthesis of one or more of the lasso peptide pathway precursors, such as amino acids.

Generally, a host microbial organism is selected such that it produces the lasso precursor peptide, either as a naturally produced molecule or as an engineered product that either provides de novo production of a desired precursor or increased production of a precursor naturally produced by the host microbial organism. For example, amino acids are produced naturally in a host organism such as E. coli. A host organism can be engineered to increase production of a lasso precursor, as disclosed herein. In addition, a microbial organism that has been engineered to produce a desirable lasso precursor peptide can be used as a host organism and further engineered to express enzymes or proteins that processes the lasso precursor peptide into matured lasso peptides.

In some embodiments, a non-naturally occurring microbial organism of the invention is generated from a host that contains the enzymatic capability to synthesize a lasso peptide. In this specific embodiment it can be useful to increase the synthesis or accumulation of a lasso peptide pathway product to, for example, drive lasso peptide pathway reactions toward lasso peptide production. Increased synthesis or accumulation can be accomplished by, for example, overexpression of nucleic acids encoding one or more of the above-described lasso peptide pathway enzymes or proteins. Over expression the enzyme or enzymes and/or protein or proteins of the lasso peptide pathway can occur, for example, through exogenous expression of the endogenous gene or genes, or through exogenous expression of the heterologous gene or genes. Therefore, naturally occurring organisms can be readily generated to be non-naturally occurring microbial organisms of the invention, for example, producing lasso peptide, through overexpression of one, two, three, four, five, six, seven, eight, nine, or ten, that is, up to all nucleic acids encoding a lasso peptide biosynthetic pathway enzymes or proteins. In addition, a non-naturally occurring organism can be generated by mutagenesis of an endogenous gene that results in an increase in activity of an enzyme in the lasso peptide biosynthetic pathway.

In particularly useful embodiments, exogenous expression of the encoding nucleic acids is employed. Exogenous expression confers the ability to custom tailor the expression and/or regulatory elements to the host and application to achieve a desired expression level that is controlled by the user. However, endogenous expression also can be utilized in other embodiments such as by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring microbial organism.

It is understood that, in methods of the invention, any of the one or more exogenous nucleic acids can be introduced into a microbial organism to produce a non-naturally occurring microbial organism of the invention. The nucleic acids can be introduced so as to confer, for example, a lasso peptide biosynthetic pathway onto the microbial organism. Alternatively, encoding nucleic acids can be introduced to produce an intermediate microbial organism having the biosynthetic capability to catalyze some of the required reactions to confer lasso peptide biosynthetic capability. For example, a non-naturally occurring microbial organism having a lasso peptide biosynthetic pathway can comprise at least one exogenous nucleic acid encoding desired enzymes or proteins, such as the linear lasso precursor peptide, or alternatively a combination of a lasso peptide peptidase and a lasso peptide cyclase. Thus, it is understood that any combination of one or more genes encoding one or more peptides, enzymes, or proteins of a biosynthetic pathway can be included in a non-naturally occurring microbial organism of the invention. Similarly, it is understood that any combination of two or more enzymes or proteins of a biosynthetic pathway can be included in a non-naturally occurring microbial organism of the invention, for example, lasso peptide peptidase and a lasso peptide cyclase, and so forth, as desired, so long as the combination of enzymes and/or proteins of the desired biosynthetic pathway results in production of the corresponding desired product.

In addition to the biosynthesis of lasso peptides as described herein, the non-naturally occurring microbial organisms and methods of the invention also can be utilized in various combinations with each other and with other microbial organisms and methods well known in the art to achieve product biosynthesis by other routes. For example, one alternative to produce a lasso peptide other than use of the lasso peptide producers is through addition of another microbial organism capable of converting a lasso peptide pathway intermediate to a lasso peptide. One such procedure includes, for example, the fermentation of a microbial organism that produces a linear lasso precursor peptide. The linear lasso precursor peptide can then be used as a substrate for a second microbial organism that converts the linear lasso precursor peptide to a lasso peptide. The linear lasso precursor peptide can be added directly to another culture of the second organism or the original culture of the linear lasso precursor peptide producers can be depleted of these microbial organisms by, for example, cell separation, and then subsequent addition of the second organism to the fermentation broth can be utilized to produce the final product without intermediate purification steps. Alternatively, a lasso peptide also can be biosynthetically produced from microbial organisms through co-culture or co-fermentation using two organisms in the same vessel, where the first microbial organism produces a linear lasso precursor peptide and the second microbial organism converts the intermediate to a lasso peptide. Alternatively, a lasso peptide also can be biosynthetically produced by first chemically synthesizing the linear lasso precursor peptide, followed by addition of the chemically synthesized linear lasso precursor peptide to a fermentation broth using one or more organisms in the same vessel, where linear lasso precursor peptide is converted to a lasso peptide. Alternatively, a lasso peptide also can be biosynthetically produced from microbial organisms through cell-free biosynthesis of the linear lasso precursor peptide, followed by addition of the linear lasso precursor peptide fermentation broth using one or more organisms in the same vessel, where linear lasso precursor peptide is converted to to a lasso peptide. Alternatively, a lasso peptide also can be biosynthetically produced by first chemically synthesizing the linear lasso precursor peptide, followed by addition of the chemically synthesized linear lasso precursor peptide to a broth containing the isolated biosynthetic enzymes, including but not limited to one or more of a lasso peptide peptidase, a lasso peptide cyclase, and lasso peptide RRE, wherein the linear lasso precursor peptide is converted to a lasso peptide. Alternatively, a lasso peptide also can be biosynthetically produced by first producing the linear lasso precursor peptide by cell-free biosynthesis methods, followed by addition of the linear lasso precursor peptide to a broth containing the isolated biosynthetic enzymes, including but not limited to one or more of a lasso peptide peptidase, a lasso peptide cyclase, and lasso peptide RRE, wherein the linear lasso precursor peptide is converted to a lasso peptide.

Given the teachings and guidance provided herein, those skilled in the art will understand that a wide variety of combinations and permutations exist for the non-naturally occurring microbial organisms and methods of the invention together with other microbial organisms, with the co-culture of other non-naturally occurring microbial organisms having subpathways and with combinations of other chemical and/or biochemical procedures well known in the art to produce a lasso peptide.

In some embodiments, the microbial organisms comprises one or more fusion protein, or a polynucleotide encoding the fusion protein such that the microbial organism is capable of producing the fusion protein through in vivo transcription and translation (TX-TL).

In some embodiments, the fusion protein comprised a lasso precursor peptide or a lasso core peptide fused to one or more additional peptide or polypeptide. In some embodiments, the one or more additional peptide or polypeptide is fused to the N-terminus of the lasso precursor peptide or lasso core peptide. In some embodiments, the one or more additional peptide or polypeptide is fused at the C-terminus of the lasso precursor peptide or lasso core peptide. In some embodiments, a polynucleotide encoding the fusion protein comprises a nucleic acid sequence encoding the lasso precursor peptide or the lasso core peptide, wherein the 5′ end of the nucleic acid sequence is linked to a nucleic acid sequence encoding the one or more additional peptide or polypeptide. In some embodiments, a polynucleotide encoding the fusion protein comprises a nucleic acid sequence encoding the lasso precursor peptide or the lasso core peptide, wherein the 3′ end of the nucleic acid sequence is linked to a nucleic acid sequence encoding the one or more additional peptide or polypeptide. In some embodiments, the fusion protein comprises an amino acid linker between the lasso precursor peptide or lasso core peptide and the one or more additional peptide or polypeptide. In some embodiments, the fusion protein does not comprise an amino acid linker between the lasso precursor peptide or lasso core peptide and the one or more additional peptide or polypeptide.

In some embodiments, the fusion protein comprised a lasso precursor peptide or a lasso core peptide fused to one or more lasso peptide biosynthesis components. In some embodiments, the one or more lasso peptide biosynthesis components are selected from (i) a lasso peptidase; (ii) a lasso cyclase; (iii) a RRE; or (iv) any combinations of (i) to (iii). In some embodiments, the one or more lasso peptide biosynthesis components are encoded by the same lasso peptide biosynthetic gene cluster. In other embodiments, the one or more lasso peptide biosynthesis components are encoded by different lasso peptide biosynthetic gene cluster.

In some embodiments, the fusion protein comprises an amino acid linker between the lasso peptidase or the lasso cyclase and the one or more additional peptide or polypeptide. In some embodiments, the fusion protein does not comprise an amino acid linker between the lasso peptidase or the lasso cyclase and the one or more additional peptide or polypeptide.

In specific embodiments, the fusion protein comprises a lasso precursor peptide fused to a lasso peptidase. In specific embodiments, the fusion protein comprises a lasso precursor peptide fused to a lasso cyclase. In specific embodiments, the fusion protein comprises a lasso precursor peptide fused to a RRE. In specific embodiments, the fusion protein comprises a lasso precursor peptide fused to a lasso peptidase and a lasso cyclase. In specific embodiments, the fusion protein comprises a lasso precursor peptide fused to a lasso peptidase and a RRE. In specific embodiments, the fusion protein comprises a lasso precursor peptide fused to a lasso cyclase and a RRE. In specific embodiments, the fusion protein comprises a lasso precursor peptide fused to a lasso peptidase, a lasso cyclase and RRE. In specific embodiments, the fusion protein comprises a lasso core peptide fused to a lasso peptidase. In specific embodiments, the fusion protein comprises a lasso core peptide fused to a lasso cyclase. In specific embodiments, the fusion protein comprises a lasso core peptide fused to a RRE. In specific embodiments, the fusion protein comprises a lasso core peptide fused to a lasso peptidase and a lasso cyclase. In specific embodiments, the fusion protein comprises a lasso core peptide fused to a lasso peptidase and a RRE. In specific embodiments, the fusion protein comprises a lasso core peptide fused to a lasso cyclase and a RRE. In specific embodiments, the fusion protein comprises a lasso core peptide fused to a lasso peptidase, a lasso cyclase and RRE.

In some embodiments, the fusion protein comprised a lasso precursor peptide or a lasso core peptide fused to one or more additional peptide or polypeptide. In some embodiments, the one or more additional peptide or polypeptide comprises a peptide or polypeptide that facilitates production of the lasso precursor peptide or lasso core peptide or the lasso peptide derived therefrom through cell-free biosynthesis. Examples of peptide or polypeptide that can be fused with a lasso precursor peptide or a lasso core peptide according to the present disclosure include but are not limited to (i) a peptide or polypeptide that increases the level of transcription of the lasso precursor peptide or lasso core peptide in the microbial organism; (ii) a peptide or polypeptide that increases the level of translation of the lasso precursor peptide or lasso core peptide in the microbial organism; (iii) a peptide or polypeptide that facilitates the processing of the lasso precursor peptide or lasso core peptide into the lasso peptide; (iv) a peptide or polypeptide that improves stability of the lasso precursor peptide or lasso core peptide or the lasso peptide derived therefrom; (v) a peptide or polypeptide that improves solubility of the lasso precursor peptide or lasso core peptide or the lasso peptide derived therefrom; (vi) a peptide or polypeptide that enables or facilitates the detection of the lasso precursor peptide or lasso core peptide or the lasso peptide derived therefrom; (vii) a peptide or polypeptide that enables or facilitates purification of the lasso precursor peptide or lasso core peptide or the lasso peptide derived therefrom; (viii) a peptide or polypeptide that enables or facilitates immobilization of the lasso precursor peptide or lasso core peptide or the lasso peptide derived therefrom; or (ix) any combination of (i) to (viii).

In some embodiments, the fusion protein comprised a lasso precursor peptide or a lasso core peptide fused to one or more additional peptide or polypeptide. In some embodiments, the one or more additional peptide or polypeptide comprises a biologically active peptide or polypeptide. Examples of biologically active peptide or polypeptide that can be fused with a lasso precursor peptide or lasso core peptide according to the present disclosure include but are not limited to (i) a peptide or polypeptide capable of binding to a target molecule (e.g., an antibody or an antigen); (ii) a peptide or polypeptide that enhance cell permeability of the fusion protein; (iii) a peptide or polypeptide capable of conjugating the fusion protein to at least one additional copy of the fusion protein; (iv) a peptide or polypeptide capable of linking the fusion protein to one or more peptidic or non-peptidic molecule; (v) a peptide or polypeptide capable of modulating activity of the lasso precursor peptide or lasso core peptide; (vi) a peptide or polypeptide capable of modulating activity of the lasso peptide derived from the lasso precursor peptide or the lasso core peptide; or (vii) any combinations of (i) to (vi).

In some embodiments, the fusion protein comprised a lasso peptidase or a lasso cyclase fused to one or more additional peptide or polypeptide. In some embodiments, the one or more additional peptide or polypeptide is fused to the N-terminus of the lasso peptidase or the lasso cyclase. In some embodiments, the one or more additional peptide or polypeptide is fused at the C-terminus of the lasso peptidase or the lasso cyclase. In some embodiments, a polynucleotide encoding the fusion protein comprises a nucleic acid sequence encoding the lasso peptidase or the lasso cyclase, wherein the 5′ end of the nucleic acid sequence is linked to a nucleic acid sequence encoding the one or more additional peptide or polypeptide. In some embodiments, a polynucleotide encoding the fusion protein comprises a nucleic acid sequence encoding the lasso peptidase or the lasso cyclase, wherein the 3′ end of the nucleic acid sequence is linked to a nucleic acid sequence encoding the one or more additional peptide or polypeptide. In some embodiments, the fusion protein comprises an amino acid linker between the lasso peptidase or the lasso cyclase and the one or more additional peptide or polypeptide. In some embodiments, the fusion protein does not comprise an amino acid linker between the lasso peptidase or the lasso cyclase and the one or more additional peptide or polypeptide.

In some embodiments, the fusion protein comprised a lasso peptidase or a lasso cyclase fused to one or more additional peptide or polypeptide. In some embodiments, the more additional peptide or polypeptide comprises a peptide or polypeptide encoded by a lasso peptide biosynthetic gene cluster. Examples of peptide or polypeptide that can be fused with a lasso precursor peptide or a lasso core peptide according to the present disclosure include but are not limited to (i) a lasso precursor peptide; (ii) a lasso core peptide; (iii) a lasso peptidase; (iv) a lasso cyclase, (v) a RRE; or (vi) any combinations of (i) to (vi). In specific embodiments, the fusion protein comprises at least one lasso cyclase and at least one lasso peptidase. In specific embodiments, the fusion protein comprises at least one lasso cyclase fused to a RRE. In specific embodiments, the fusion protein comprises at least one lasso peptidase fused to a RRE.

In some embodiments, the fusion protein comprised a lasso peptidase or a lasso cyclase fused to one or more additional peptide or polypeptide. In some embodiments, the one or more additional peptide or polypeptide comprises a peptide or polypeptide that facilitates production of the lasso peptidase or lasso cyclase through cell-free biosynthesis. Examples of peptide or polypeptide that can be fused with the lasso peptidase or lasso cyclase according to the present disclosure include but are not limited to (i) a peptide or polypeptide that increases the level of transcription of the lasso peptidase or lasso cyclase in the microbial organism; (ii) a peptide or polypeptide that increases the level of translation of the lasso peptidase or lasso cyclase in the microbial organism; (iii) a peptide or polypeptide that improves stability of the lasso peptidase or lasso cyclase; (vi) a peptide or polypeptide that improves solubility of the lasso peptidase or lasso cyclase; (v) a peptide or polypeptide that enables or facilitates the detection of the lasso peptidase or lasso cyclase; (vi) a peptide or polypeptide that enables or facilitates purification of the lasso peptidase or lasso cyclase; (vii) a peptide or polypeptide that enables or facilitates immobilization of the lasso peptidase or lasso cyclase; or (viii) any combination of (i) to (vii).

In some embodiments, the fusion protein comprised a lasso peptidase or a lasso cyclase fused to one or more additional peptide or polypeptide. In some embodiments, the one or more additional peptide or polypeptide comprises a biologically active peptide or polypeptide. Examples of biologically active peptide or polypeptide that can be fused with a lasso peptidase or a lasso cyclase according to the present disclosure include but are not limited to (i) a peptide or polypeptide capable of modulating the reaction catalyzing activity of the lasso peptidase or lasso cyclase; (ii) a peptide or polypeptide capable of modulating target specificity of the lasso peptidase or lasso cyclase; (iii) an enzyme having the same or different enzymatic activity as the lasso peptidase or lasso cyclase; or any combination of (i) to (iii).

In some embodiments, the fusion protein comprised a RIPP recognition element (RRE) fused to one or more additional peptide or polypeptide. In some embodiments, the one or more additional peptide or polypeptide is fused to the N-terminus of the RRE. In some embodiments, the one or more additional peptide or polypeptide is fused at the C-terminus of the RRE. In some embodiments, a polynucleotide encoding the fusion protein comprises a nucleic acid sequence encoding the RRE, wherein the 5′ end of the nucleic acid sequence is linked to a nucleic acid sequence encoding the one or more additional peptide or polypeptide. In some embodiments, a polynucleotide encoding the fusion protein comprises a nucleic acid sequence encoding the RRE, wherein the 3′ end of the nucleic acid sequence is linked to a nucleic acid sequence encoding the one or more additional peptide or polypeptide. In some embodiments, the fusion protein comprises an amino acid linker between the RRE and the one or more additional peptide or polypeptide. In some embodiments, the fusion protein does not comprise an amino acid linker between RRE and the one or more additional peptide or polypeptide.

In some embodiments, the fusion protein comprised a RIPP recognition element (RRE) fused to one or more additional peptide or polypeptide. In some embodiments, the more additional peptide or polypeptide comprises a peptide or polypeptide encoded by a lasso peptide biosynthetic gene cluster. Examples of peptide or polypeptide that can be fused with a lasso precursor peptide or a lasso core peptide according to the present disclosure include but are not limited to (i) a lasso precursor peptide; (ii) a lasso core peptide; (iii) a lasso peptidase; (iv) a lasso cyclase, (v) a RRE; or (vi) any combinations of (i) to (vi). In specific embodiments, the fusion protein comprises at least one lasso precursor peptide fused to a RRE. In specific embodiments, the fusion protein comprises at least one lasso core peptide fused to a RRE. In specific embodiments, the fusion protein comprises at least one lasso cyclase fused to a RRE. In specific embodiments, the fusion protein comprises at least one lasso peptidase fused to a RRE.

In some embodiments, the fusion protein comprised a RIPP recognition element (RRE) fused to one or more additional peptide or polypeptide. In some embodiments, the one or more additional peptide or polypeptide comprises a peptide or polypeptide that facilitates production of the RRE through cell-free biosynthesis. Examples of peptide or polypeptide that can be fused with the RRE according to the present disclosure include but are not limited to (i) a peptide or polypeptide that increases the level of transcription of the RRE in the microbial organism; (ii) a peptide or polypeptide that increases the level of translation of the RRE in the microbial organism; (iii) a peptide or polypeptide that improves stability of the RRE; (vi) a peptide or polypeptide that improves solubility of the RRE; (v) a peptide or polypeptide that enables or facilitates the detection of the RRE; (vi) a peptide or polypeptide that enables or facilitates purification of the RRE; (vii) a peptide or polypeptide that enables or facilitates immobilization of the RRE; or (viii) any combination of (i) to (vii).

In some embodiments, the fusion protein comprised a RIPP recognition element (RRE) fused to one or more additional peptide or polypeptide. In some embodiments, the one or more additional peptide or polypeptide comprises a biologically active peptide or polypeptide. Examples of biologically active peptide or polypeptide that can be fused with a RRE according to the present disclosure include but are not limited to (i) a peptide or polypeptide capable of modulating the reaction catalyzing activity of the lasso peptidase or lasso cyclase; (ii) a peptide or polypeptide capable of modulating target specificity of the lasso peptidase or lasso cyclase; (iii) an enzyme having the same or different enzymatic activity as the lasso peptidase or lasso cyclase; or any combination of (i) to (iii).

In particular embodiments, the lasso precursor peptide genes are fused at the 5′-terminus of the DNA template strand of the gene to oligonucleotide sequences that encode peptides or proteins, such as sequences encoding maltose-binding protein (MBP) or small ubiquitin-like modifier protein (SUMO), which enhance the stability, solubility, and production of the desired TX-TL products (Marblestone, J. G., et al., Protein Sci, 2006, 15, 182-189). In particular embodiments, the lasso precursor peptides are fused at the C-terminus of the leader sequences to form conjugates with peptides or proteins, such as maltose-binding protein or small ubiquitin-like modifier protein, which enhance the stability, solubility, and production of the fused MBP-lasso or SUMO-lasso precursor peptide.

In particular embodiments, the lasso precursor peptide genes or lasso core peptide genes are fused at the 3′-terminus of the DNA template strand of the gene to oligonucleotide sequences that encode peptides or proteins, such as sequences encoding maltose-binding protein (MBP) or small ubiquitin-like modifier protein (SUMO), which enhance the stability, solubility, and production of the desired TX-TL products. In particular embodiments, the lasso precursor peptides, lasso core peptides, or lasso peptides are fused at the N-terminus to form conjugates with peptides or proteins, such as maltose-binding protein or small ubiquitin-like modifier protein, which enhance the stability, solubility, and production of the fused MBP-lasso or SUMO-lasso precursor peptide.

In particular embodiments, the lasso precursor peptide genes or lasso core peptide genes are fused at the 5′-terminus of the DNA template strand of the gene to oligonucleotide sequences that encode peptides or proteins, with or without a linker, such as sequences encoding peptide tags for affinity purification or immobilization, including his-tags, a strep-tags, or FLAG-tags. In some embodiments, the lasso precursor peptides, lasso core peptides, or lasso peptides are fused at the C-terminus of the core peptides to form conjugates with other peptides or proteins, with or without a linker, such as peptide tags for affinity purification or immobilization, including his-tags, a strep-tags, or FLAG-tags.

In particular embodiments, lasso precursor peptides, lasso core peptides, or lasso peptides are fused to molecules that can enhance cell permeability or penetration into cells, for example through the use of arginine-rich cell-penetrating peptides such as TAT peptide, penetratin, and flock house virus (FHV) coat peptide (Brock, R., Bioconjug. Chem., 2014, 25, 863-868). In particular embodiments, a lasso precursor peptide gene or core peptide gene is fused at the 3′-terminus to oligonucleotide sequences that encode arginine-rich cell-penetrating peptides or proteins, including oligonucleotide sequences that encode penetratin, and flock house virus (FHV) coat peptide or similar peptides that contain guanidinium groups or a combination of lysine and guanidinium groups (Wender, P. A., et al., Adv. Drug Deliv. Rev., 2008, 60, 452-472). In particular embodiments, a lasso precursor peptide, lasso core peptide, or lasso peptide is fused at the C-terminus to peptides that promote cell penetration such as arginine-rich cell-penetrating peptides or proteins, including amino acid sequences that encode TAT peptide, penetratin, and flock house virus (FHV) coat peptide or similar peptides that contain guanidinium groups or a combination of lysine and guanidinium groups.

In particular embodiments, the lasso precursor peptide genes or lasso core peptide genes are fused at the 5′-terminus of the DNA template strand of the gene to oligonucleotide sequences that encode peptides or proteins, with or without a linker, such as sequences encoding peptide epitopes that are known to bind with high affinity to antibodies, cell surface proteins, or cell surface receptors, including cytokine binding epitopes, integrin ligand binding epitopes, and the like. In particular embodiments, the lasso precursor peptides, lasso core peptides, or lasso peptides are fused at the C-terminus to peptides or proteins, with or without a linker, such as peptide epitopes that are known to bind with high affinity to antibodies, cell surface proteins, or cell surface receptors, including cytokine binding epitopes, integrin ligand binding epitopes, and the like.

In specific embodiments, the method comprises (a) introducing into the microbial organism a first nucleic acid sequence encoding a peptide comprising a sequence of SEQ ID NOS:1-34 and 42-71 and a second nucleic acid sequence encoding a lasso peptide biosynthesis component; and (b) culturing the microbial organism under a condition suitable for lasso formation to produce the lasso peptide. In some embodiments, the method comprises (a) introducing into the microbial organism a first nucleic acid sequence encoding a peptide comprising a sequence of SEQ ID NOS:1-17 and 42-56 and a second nucleic acid sequence encoding a lasso peptide biosynthesis component comprising a lasso cyclase; and (b) culturing the microbial organism under a condition suitable for lasso formation to produce the lasso peptide. In some embodiments, the method comprises (a) introducing into the microbial organism a first nucleic acid sequence encoding a peptide comprising a sequence of SEQ ID NOS:18-34 and 57-71 and a second nucleic acid sequence encoding a lasso peptide biosynthesis component comprising a lasso peptidase and a lasso cyclase; and (b) culturing the microbial organism under a condition suitable for lasso formation to produce the lasso peptide. Particularly, in some embodiments, the lasso peptidase comprises a sequence of SEQ ID NO:35. In some embodiments, the lasso cyclase comprises a sequence of SEQ ID NO:36. In some embodiments, the lasso cyclase comprises a sequence of SEQ ID NO:37. In some embodiments, the lasso peptide comprises an amino acid sequence selected from SEQ ID NOS:1-17 and 42-56 is G1-D9 cyclized.

In specific embodiments, the lasso peptide biosynthesis component comprises a lasso cyclase. In those embodiments where the lasso peptide biosynthesis component comprises a lasso peptidase and a lasso cyclase, the method comprises introducing the second nucleic acid sequence encoding the lasso cyclase and a third nucleic acid sequence encoding the lasso peptidase. In those embodiments where the lasso peptide biosynthesis component comprises a lasso cyclase and a post-translationally modified peptide (RiPP) recognition element (RRE), the method comprises introducing the second nucleic acid sequence encoding the lasso cyclase and a fourth nucleic acid sequence encoding the RRE. In those embodiments where the lasso peptide biosynthesis component comprises a lasso peptidase, a lasso cyclase and a post-translationally modified peptide (RiPP) recognition element (RRE), and the method comprises introducing the second nucleic acid sequence encoding the lasso cyclase, a third nucleic acid sequence encoding the lasso peptidase, and a fourth nucleic acid sequence encoding the RRE. Particularly, in some embodiments, the lasso peptidase comprises a sequence of SEQ ID NO:35. In some embodiments, the lasso cyclase comprises a sequence of SEQ ID NO:36. In some embodiments, the lasso cyclase comprises a sequence of SEQ ID NO:37. In some embodiments, at least two of the first, second, third and fourth nucleic acid sequences are in a same nucleic acid molecule. In some embodiments, the lasso peptide comprises an amino acid sequence selected from SEQ ID NOS:1-17 and 42-56 is G1-D9 cyclized.

In some embodiments, to produce lasso peptides having amino add sequences selected from SEQ ID NOS:1-17 and 42-56, the core peptide sequences (SEQ ID NOS:1-17 and 42-56) can be fused to any known leader sequence, thereby producing a lasso precursor peptide, and the method then employs one or more lasso peptide biosynthesis component capable of recognizing such leader sequence and processing the lasso precursor peptide into matured lasso peptides having SEQ ID NOS:1-17 and 42-56.

Accordingly, in some embodiments, provided herein is a method of producing a lasso peptide having an amino acid sequence selected from SEQ ID NOS:1-17 and 42-56, wherein the method comprises (a) introducing into a microbial organism a first nucleic acid sequence encoding a lasso precursor peptide comprising a leader sequence and a lasso core peptide sequence selected from SEQ ID NOS:1-17 and 42-56 and a second nucleic acid sequence encoding a lasso peptide biosynthesis component; and (b) culturing the microbial organism under a condition suitable for lasso formation to produce the lasso peptide; wherein the lasso peptide biosynthesis component comprises a lasso peptidase capable of catalyzing removal of the leader sequence. Particularly, in these embodiments, the corresponding leader sequence and lasso peptide biosynthesis components can be those disclosed in PCT application publication number: WO2019/191571. In some embodiments, at least two of the first, second, third and fourth nucleic acid sequences are in a same nucleic acid molecule. In some embodiments, the lasso peptide comprises an amino acid sequence selected from SEQ ID NOS:1-17 and 42-56 is G1-D9 cyclized.

Any of the non-naturally occurring microbial organisms described herein can be cultured to produce and/or secrete the biosynthetic products of the invention. For example, the lasso peptide producers can be cultured for the biosynthetic production of lasso peptide. For example, host microbial organisms can be selected from, and the non-naturally occurring microbial organisms generated in, for example, bacteria, yeast, fungus or any of a variety of other microorganisms applicable to fermentation processes. Exemplary bacteria include species selected from Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Streptomyces albus, Clostridium acetobutylicum, Vibrio natriegens, Pseudomonas fluorescens, and Pseudomonas putida. Exemplary yeasts or fungi include species selected from Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, and the like. E. coli is a particularly useful host organisms since it is a well characterized microbial organism suitable for genetic engineering. Other particularly useful host organisms include yeast such as Saccharomyces cerevisiae. Other particularly useful microbial organisms for the cell-based biosynthesis of lasso peptide include, for example, Vibrio natriegens, Burholderia spp., Corynebacterium glutamicum, or Sphingomaons subterranean.

Sources of encoding nucleic acids for a lasso peptide pathway enzyme or protein can include, for example, any species where the encoded gene product is capable of catalyzing the referenced reaction. Such species include both prokaryotic organisms including, but not limited to, bacteria, including archaea and eubacteria, Exemplary species for such sources include, for example, Escherichia coli, Acidaminococcus fermentans, Acinetobacter baylyi, Acinetobacter calcoaceticus, Acinetobacter sp. ADP1, Acinetobacter sp. Strain M-1, Aquifex aeolicus, Arabidopsis thaliana, Arabidopsis thaliana col, Arabidopsis thaliana col, Archaeoglobus fulgidus DSM 4304, Azoarcus sp. CIB, Bacillus cereus, Bacillus subtilis, Bos Taurus, Brucella melitensis, Burkholderia ambifaria AMID, Burkholderia phymatum, Campylobacter jejuni, Chloroflexus aurantiacus, Citrobacter youngae ATCC 29220, Clostridium acetobutylicum, Clostridium aminobutyricum, Clostridium beijerinckii, Clostridium beijerinckii NCIMB 8052, Clostridium beijerinckii NRRL B593, Clostridium botulinum C str. Eklund, Clostridium kluyveri, Clostridium kluyveri DSM 555, Clostridium novyi NT, Clostridium propionicum, Clostridium saccharoperbutylacetonicum, Corynebacterium glutamicum ATCC 13032, Cupriavidus taiwanensis, Cyanobium PCC7001, Dictyostelium discoideum AX4, Enterococcus faecalis, Erythrobacter sp. NAP1, Escherichia coli K12, Escherichia coli str. K-12 substr. MG1655, Eubacterium rectale ATCC 33656, Fusobacterium nucleatum, Fusobacterium nucleatum subsp. nucleatum ATCC 25586, Geobacillus thermoglucosidasius, Haematococcus pluvialis, Haemophilus influenzae, Haloarcula marismortui ATCC 43049, Helicobacter pylori, Homo sapiens, Klebsiella pneumoniae, Lactobacillus plantarum, Leuconostoc mesenteroides, marine gamma proteobacterium HTCC2080, Metallosphaera sedula, Methanocaldococcus jannaschii, Mus musculus, Mycobacterium avium subsp. paratuberculosis K-10, Mycobacterium bovis BCG, Mycobacterium marinum M, Mycobacterium smegmatis MC2 155, Mycobacterium tuberculosis, Mycoplasma pneumoniae M129, Nocardia farcinica IFM 10152, Nocardia iowensis (sp. NRRL 5646), Oryctolagus cuniculus, Paracoccus denitrificans, Penicillium chrysogenum, Porphyromonas gingivalis, Porphyromonas gingivalis W83, Pseudomonas aeruginosa, Pseudomonas aeruginosa PAO1, Pseudomonas fluorescens, Pseudomonas fluorescens Pf-5, Pseudomonas knackmussii (B13), Pseudomonas putida, Pseudomonas putida E23, Pseudomonas putida KT2440, Pseudomonas sp, Pyrobaculum aerophilum str. IM2, Pyrococcus furiosus, Ralstonia eutropha, Ralstonia eutropha H16, Ralstonia eutropha H16, Ralstonia metallidurans, Rattus norvegicus, Rhodobacter spaeroides, Rhodococcus rubber, Rhodopseudomonas palustris, Roseburia intestinalis LI-82, Roseburia inulinivorans DSM 16841, Roseburia sp. A2-183, Roseiflexus castenholzii, NRRL 2338, Salmonella enterica subsp. arizonae serovar, Salmonella typhimurium, Schizosaccharomyces pombe, Simmondsia chinensis, Sinorhizobium meliloti, Staphylococcus aureus, Streptococcus pneumoniae, Streptomyces coelicolor, Streptomyces griseus subsp. griseus, BRC 13350, Streptomyces sp. ACT-1, Sulfolobus acidocaldarius, Sulfolobus shibatae, Sulfolobus solfataricus, Sulfolobus tokodaii, Synechocystis sp. strain PCC6803, Syntrophus, ciditrophicus, Thermoanaerobacter brockii HTD4, Thermoanaerobacter tengcongensis MB4, Thermosynechococcus elongates, Thermotoga maritime MSB8, Thermus thermophilus, Thermus, hermophilus HB8, Trichomonas vaginalis G3, Trichosporonoides megachiliensis, Trypanosoma brucei, Tsukamurella paurometabola DSM 20162, Vibrio natriegens, Yersinia intermedia ATCC 29909, Zoogloea ramigera, Zygosaccharomyces rouxii, Zymomonas mobilis, as well as other exemplary species disclosed herein are available as source organisms for corresponding genes. However, with the complete genome sequence available for now more than 1000 bacterial species, the identification of genes encoding the requisite lasso peptide biosynthetic activity for one or more genes in related or distant species, including for example, homologues, orthologs, paralogs and nonorthologous gene displacements of known genes, and the interchange of genetic alterations between organisms is routine and well known in the art. Accordingly, the metabolic alterations allowing biosynthesis of a lasso peptide described herein with reference to a particular organism such as E. coli can be readily applied to other microorganisms. Given the teachings and guidance provided herein, those skilled in the art will know that a metabolic alteration exemplified in one organism can be applied equally to other organisms.

In some instances, such as when an alternative lasso peptide biosynthetic pathway exists in an unrelated species, lasso peptide biosynthesis can be conferred onto the host species by, for example, exogenous expression of a paralog or paralogs from the unrelated species that catalyzes a similar, yet non-identical metabolic reaction to replace the referenced reaction. Because certain differences among metabolic networks exist between different organisms, those skilled in the art will understand that the actual gene usage between different organisms may differ. However, given the teachings and guidance provided herein, those skilled in the art also will understand that the teachings and methods of the invention can be applied to all microbial organisms using the cognate metabolic alterations to those exemplified herein to construct a microbial organism in a species of interest that will synthesize a lasso peptide.

Methods for constructing and testing the expression levels of a non-naturally occurring lasso peptide-producing host can be performed, for example, by recombinant and detection methods well known in the art. Such methods can be found described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999).

Exogenous nucleic acid sequences involved in a pathway for production of a lasso peptide can be introduced stably or transiently into a host cell using techniques well known in the art including, but not limited to, conjugation, electroporation, chemical transformation, transduction, transfection, and ultrasound transformation. For exogenous expression in E. coli or other prokaryotic cells, some nucleic acid sequences in the genes can encode targeting signals such as an N-terminal mitochondrial or other targeting signal, which can be removed before transformation into prokaryotic host cells, if desired. Furthermore, genes can be subjected to codon optimization with techniques well known in the art to achieve optimized expression of the active proteins.

An expression vector or vectors can be constructed to include one or more lasso peptide biosynthetic pathway encoding nucleic acids as exemplified herein operably linked to expression control sequences functional in the host organism. Expression vectors applicable for use in the microbial host organisms of the invention include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

For the production of lasso peptide, the recombinant strains are cultured in a medium with carbon source and other essential nutrients. It is sometimes desirable and can be highly desirable to maintain anaerobic conditions in the fermenter to reduce the cost of the overall process. Such conditions can be obtained, for example, by first sparging the medium with nitrogen and then sealing the flasks with a septum and crimp-cap. For strains where growth is not observed anaerobically, microaerobic or substantially anaerobic conditions can be applied by perforating the septum with a small hole for limited aeration. Exemplary anaerobic conditions have been described previously and are well-known in the art. Exemplary aerobic and anaerobic conditions are described, for example, in United State publication 2009/0047719, filed Aug. 10, 2007. Fermentations can be performed in a batch, fed-batch or continuous manner, as disclosed herein.

The growth medium can include, for example, any carbohydrate source which can supply a source of carbon to the non-naturally occurring microorganism. Such sources include, for example, sugars such as glucose, xylose, arabinose, galactose, mannose, fructose, sucrose and starch. Other sources of carbohydrate include, for example, renewable feedstocks and biomass. Exemplary types of biomasses that can be used as feedstocks in the methods of the invention include cellulosic biomass, hemicellulosic biomass and lignin feedstocks or portions of feedstocks. Such biomass feedstocks contain, for example, carbohydrate substrates useful as carbon sources such as glucose, xylose, arabinose, galactose, mannose, fructose and starch. Given the teachings and guidance provided herein, those skilled in the art will understand that renewable feedstocks and biomass other than those exemplified above also can be used for culturing the microbial organisms of the invention for the production of a lasso peptide.

In various embodiments, the cell-free or cell-based biosynthesis system (e.g., a CFB reaction mixture of cell culture) can be maintained under aerobic or substantially aerobic conditions, where such conditions can be achieved, for example, by sparging with air or oxygen, shaking under an atmosphere of air or oxygen, stirring under an atmosphere of air or oxygen, or combinations thereof.

In alternative embodiments, the cell-free or cell-based biosynthesis system (e.g., a CFB reaction mixture of cell culture) can be maintained under anaerobic or substantially anaerobic conditions, where such conditions can be achieved, for example, by first sparging the medium with nitrogen and then sealing the wells or reaction containers, or by shaking or stirring under a nitrogen atmosphere. Briefly, anaerobic conditions refer to an environment devoid of oxygen. Substantially anaerobic conditions include, for example, biosynthesis processes conducted such that the dissolved oxygen concentration in the medium remains between 0 and 10% of saturation. Substantially anaerobic conditions also include performing the biosynthesis methods and processes inside a sealed chamber maintained with an atmosphere of less than 1% oxygen. The percent of oxygen can be maintained by, for example, sparging the CFB reaction or cell culture with an N₂/CO₂ mixture or other suitable non-oxygen gas or gases.

If desired, the pH of the cell culture medium or CFB reaction mixture, including cell extracts, used in the biosynthesis methods and systems, provided herein for the synthesis of lasso peptides and related molecules thereof can be maintained at a desired pH, in particular neutral pH, such as a pH of around 7 by addition of a buffer, a base, such as NaOH or other bases, or an acid, as needed to maintain the production system at a desirable pH for high rates and yields in the production of lasso peptides and related molecules thereof.

In alternative embodiments, the cell culture medium or CFB reaction mixture, including cell extracts, used in the CFB methods and systems, provided herein for the synthesis of lasso peptides and related molecules thereof can be supplemented with one or more enzymes (or the nucleic acids that encode them) of central metabolism pathways, for example, one or more (or all of the) central metabolism enzymes from the tricarboxylic acid cycle (TCA, or Krebs cycle), the glycolysis pathway or the Citric Acid Cycle, or enzymes that promote the production of amino acids.

Metabolic modeling and simulation algorithms can be utilized to optimize conditions for the present biosynthesis process and to optimize lasso peptide production rates and yields in the cell-free or cell-based system. Modeling can also be used to design gene knockouts that additionally optimize utilization of the lasso peptide pathway (see, for example, U.S. patent publications US 2002/0012939, US 2003/0224363, US 2004/0029149, US 2004/0072723, US 2003/0059792, US 2002/0168654 and US 2004/0009466, and U.S. Pat. No. 7,127,379). Modeling analysis allows reliable predictions of the effects on shifting the primary metabolism towards more efficient production of lasso peptides and related molecules thereof.

One computational method for identifying and designing metabolic alterations favoring biosynthesis of a desired product is the OptKnock computational framework (Burgard et al., Biotechnol. Bioeng., 2003, 84, 647-657). OptKnock is a metabolic modeling and simulation program that suggests gene deletion or disruption strategies that result in genetically stable metabolic network which overproduces the target product. Specifically, the framework examines the complete metabolic and/or biochemical network in order to suggest genetic manipulations that lead to maximum production of a lasso peptide or related molecules thereof. Such genetic manipulations can be performed on strains used to produce cell extracts for the CFB methods and processes provided herein. Also, this computational methodology can be used to either identify alternative pathways that lead to biosynthesis of a desired lasso peptide or used in connection with non-naturally occurring systems for further optimization of biosynthesis of a desired lasso peptide.

Briefly, OptKnock is a term used herein to refer to a computational method and system for modeling cellular metabolism. The OptKnock program relates to a framework of models and methods that incorporate particular constraints into flux balance analysis (FBA) models. These constraints include, for example, qualitative kinetic information, qualitative regulatory information, and/or DNA microarray experimental data. OptKnock also computes solutions to various metabolic problems by, for example, tightening the flux boundaries derived through flux balance models and subsequently probing the performance limits of metabolic networks in the presence of gene additions or deletions. OptKnock computational framework allows the construction of model formulations that allow an effective query of the performance limits of metabolic networks and provides methods for solving the resulting mixed-integer linear programming problems. The metabolic modeling and simulation methods referred to herein as OptKnock are described in, for example, U.S. publication 2002/0168654, filed Jan. 10, 2002, in International Patent No. PCT/US02/00660, filed Jan. 10, 2002, and U.S. publication 2009/0047719, filed Aug. 10, 2007.

Another computational method for identifying and designing metabolic alterations favoring biosynthetic production of a product is a metabolic modeling and simulation system termed SimPheny®. This computational method and system is described in, for example, U.S. publication 2003/0233218, filed Jun. 14, 2002, and in International Patent Application No. PCT/US03/18838, filed Jun. 13, 2003. SimPheny® is a computational system that can be used to produce a network model in silico and to simulate the flux of mass, energy or charge through the chemical reactions of a biological system to define a solution space that contains any and all possible functionalities of the chemical reactions in the system, thereby determining a range of allowed activities for the biological system. This approach is referred to as constraints-based modeling because the solution space is defined by constraints such as the known stoichiometry of the included reactions as well as reaction thermodynamic and capacity constraints associated with maximum fluxes through reactions. The space defined by these constraints can be interrogated to determine the phenotypic capabilities and behavior of the biological system or of its biochemical components.

These computational approaches are consistent with biological realities because biological systems are flexible and can reach the same result in different ways. Biological systems are designed through evolutionary mechanisms that have been restricted by fundamental constraints that all living systems must face. Therefore, constraints-based modeling strategy embraces these general realities. Further, the ability to continuously impose further restrictions on a network model via the tightening of constraints results in a reduction in the size of the solution space, thereby enhancing the precision with which biosynthetic performance can be predicted.

Given the teachings and guidance provided herein, those skilled in the art will be able to apply various computational frameworks for metabolic modeling and simulation to design and implement biosynthesis of lasso peptides or related molecules thereof using whole cells or cell extracts and the biosynthesis methods and processes provided herein for the production of lasso peptides and related molecules thereof. Such metabolic modeling and simulation methods include, for example, the computational systems exemplified above as SimPheny® and OptKnock. Those skilled in the art will know how to apply the identification, design and implementation of the metabolic alterations using OptKnock to any of such other metabolic modeling and simulation computational frameworks and methods well known in the art.

Suitable purification and/or assays to test for the production of lasso peptides or functional fragments of lasso peptides can be performed using well known methods. Suitable replicates such as triplicate CFB reactions or cell cultures, can be conducted and analyzed to verify lasso peptide production and concentrations. The final product of lasso peptides, functional fragments of lasso peptides, intermediates, and other organic compounds, can be analyzed by methods such as HPLC (High Performance Liquid Chromatography), GC-MS (Gas Chromatography-Mass Spectrometry), LC-MS (Liquid Chromatography-Mass Spectrometry), MALDI or other suitable analytical methods using routine procedures well known in the art. Byproducts and residual amino acids or glucose can be quantified by HPLC using, for example, a refractive index detector for glucose and saturated fatty acids, and a UV detector for amino acids and other organic acids (Lin et al., Biotechnol. Bioeng., 2005, 90, 775-779), or other suitable assay and detection methods well known in the art. The individual enzyme or protein activities encoded by exogenous or endogenous DNA sequences can also be assayed using methods well known in the art.

Biosynthesized peptide or polypeptide can be isolated, separated purified from other components in the CFB reaction mixtures or cell culture medium using a variety of methods well known in the art. Such separation methods include, for example, extraction procedures, including extraction of CFB reaction mixtures using organic solvents such as methanol, butanol, ethyl acetate, and the like, as well as methods that include continuous liquid-liquid extraction, solid-liquid extraction, solid phase extraction, pervaporation, membrane filtration, membrane separation, reverse osmosis, electrodialysis, dialysis, distillation, crystallization, centrifugation, extractive filtration, ion exchange chromatography, size exclusion chromatography, adsorption chromatography, ultrafiltration, medium pressure liquid chromatography (MPLC), and high pressure liquid chromatography (HPLC). All of the above methods are well known in the art and can be implemented in either analytical or preparative modes.

5.4.3 Composition

In a related aspect of the present disclosure, provided herein are compositions comprising the endothelin B receptor (ETBR) antagonistic lasso peptides of the present disclosure. In some embodiments, the composition comprises lasso peptides that is biosynthesized using the present cell-free or cell-based methods described in above Section 5.4.2.

In some embodiments, the composition comprises an effective amount of at least one lasso peptide and pharmaceutically acceptable carrier(s) or excipient(s). In some embodiments, the composition further comprises an effective amount of at least one additional therapeutic agent that is not a lasso peptide.

In some embodiments, the additional therapeutic agent can be a chemotherapeutic agent, such as one or more of cyclophosphamide, thiotepa, mechlorethamine (chlormethine/mustine), uramustine, melphalan, chlorambucil, ifosfamide, chlornaphazine, cholophosphamide, estramustine, novembichin, phenesterine, prednimustine, trofosfamide, uracil mustard, bendamustine, busulfan, improsulfan, piposulfan, carmustine, lomustine, chlorozotocin, fotemustine, nimustine, ranimustine, streptozucin, cisplatin, carboplatin, nedaplatin, oxaliplatin, satraplatin, triplatin tetranitrate, procarbazine, altretamine, dacarbazine, mitozolomide, temozolomide, paclitaxel, docetaxel, vinblastine, vincristine, vinorelbine, cabazitaxel, dactinomycin (actinomycin D), calicheamicin, dynemicin, amsacrine, doxarubicin, daunorubicin, epirubicin, mitoxantrone, idarubicin, pirarubicin, benzodopa, carboquone, meturedopa, uredopa, altretamine, triethylenemelamine, trietylenephosphoramide, triethiylenethiophosphoramide, trimethylolomelamine, bullatacin, bullatacinone, camptothecin, topotecan, bryostatin, callystatin, CC-1065, adozelesin, carzelesin, bizelesin, cryptophycin, dolastatin, duocarmycin, KW-2189, CB1-TM1, eleutherobin, pancratistatin, sarcodictyin, spongistatin, clodronate, esperamicin, neocarzinostatin chromophore, aclacinomysin, anthramycin, azaserine, bleomycin, cactinomycin, carabicin, carminomycin, carzinophilin, chromomycinis, detorubicin, 6-diazo-5-oxo-L-norleucine, esorubicin, idarubicin, marcellomycin, mitomycin, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, mitotane, trilostane, frolinic acid, aceglatone, aldophosphamide glycoside, aminolevulinic acid, eniluracil, bestrabucil, bisantrene, edatraxate, defofamine, demecolcine, diaziquone, elformithine, elliptinium acetate, etoglucid, gallium nitrate, hydroxyurea, lentinan, lonidainine, maytansine, ansamitocins, mitoguazone, mopidanmol, nitraerine, pentostatin, phenamet, pirarubicin, losoxantrone, podophyllinic acid, 2-ethylhydrazide, PSK polysaccharide complex, razoxane, rhizoxin, sizofiran, spirogermanium, tenuazonic acid, triaziquone, 2,2′,2″-trichlorotriethylamine; T-2 toxin, verracurin A, roridin A and anguidine, urethan, vindesine, mannomustine, mitobronitol, mitolactol, pipobroman, gacytosine, arabinoside (“Ara-C”), etoposide (VP-16), vinorelbine, novantrone, teniposide, edatrexate, aminopterin, xeloda, ibandronate, irinotecan (e.g., CPT-11), topoisomerase inhibitor RFS 2000, difluorometlhylornithine (DMFO), retinoic acid, capecitabine, plicomycin, gemcitabine, navelbine, transplatinum, and pharmaceutically acceptable salts, acids, or derivatives of any of the above. In specific embodiments, the chemotherapeutic agent comprises cyclophosphamide.

In some embodiments, the additional therapeutic agent can be a immunotherapeutic agent, such as one or more of immune checkpoint modulator that inhibits, decreases or interferes with the activity of a negative checkpoint regulator. In certain embodiments, the negative checkpoint regulator is selected from Cytotoxic T-lymphocyte antigen-4 (CTLA-4), CD40, CD47, CD80, CD86, Programmed cell death 1 (PD-1), Programmed cell death ligand 1 (PD-L1), Programmed cell death ligand 2 (PD-L2), Lymphocyte activation gene-3 (LAG-3; also known as CD223), Galectin-3, B and T lymphocyte attenuator (BTLA), T-cell membrane protein 3 (TIM3), Galectin-9 (GALS), B7-H1, B7-H3, B7-H4, T-Cell immunoreceptor with Ig and ITIM domains (TIGIT/Vstm3/WUCAM/VSIG9), V-domain Ig suppressor of T-Cell activation (VISTA), Glucocorticoid-induced tumor necrosis factor receptor-related (GITR) protein, Herpes Virus Entry Mediator (HVEM), OX40, CD27, CD28, CD137. CGEN-15001T, CGEN-15022, CGEN-15027, CGEN-15049, CGEN-15052, and CGEN-15092. In certain embodiments, the immune checkpoint inhibitor is an anti-PD-1 antibody. In certain embodiments, the immune checkpoint inhibitor is an anti-PD-L1 antibody.

In some embodiments, the additional therapeutic agent can be a cancer vaccine, such as sipuleucel-T vaccine, Bacillus Calmette-Guérin vaccine, LLO-E7 DNA vaccine, and T-VEC (Imlygic®).

The compositions provided herein can be formulated for administration via a suitable route of administration, for example, for oral, nasal, rectal, topical (including transdermal), ocular, intracerebral, intracranial, intrathecal, intra-arterial, intravenous, intramuscular, or other parental routes of administration. Depending upon the selection of a particular route of administration, pharmaceutically-acceptable carriers well-known in the art may be used.

The compositions provided herein may be formulated in a pharmaceutically acceptable formulation forms. Selection of a proper formulation of a pharmaceutical composition can depend the route of administration chosen. A summary of pharmaceutical compositions is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

For example, suitable forms of formulations described herein include, but are not limited to, aqueous liquid dispersions, self-emulsifying dispersions, solid solutions, liposomal dispersions, aerosols, solid dosage forms, powders, immediate release formulations, controlled release formulations, fast smelt formulations, tablets, capsules, pills, delayed release formulations, extended release formulations, pulsatile release formulations, multi-particulate formulations, and mixed immediate and controlled release formulations. The pharmaceutical compositions will include at least one lasso peptide, as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these lasso peptides having the same type of activity.

The compositions described herein can be provided in unit dosage form. A unit dosage form can be a composition containing an amount of a compound that is suitable for administration to a subject (such as a human), in a single dose unit, according to good medical practice. The preparation of a single unit dosage form, however, does not imply that the dosage form is administered once per day or once per course of therapy. Such dosage forms are contemplated to be administered once, twice, thrice or more per day and multiple unit dosage forms may be administered at one time, though a single administration is not specifically excluded. The skilled artisan will recognize that the formulation does not specifically contemplate the entire course of therapy and such decisions are left for those skilled in the art of treatment rather than formulation.

The amount of the lasso peptide and the additional therapeutic agent (where applicable) can vary depending upon the subject being treated and the particular mode of administration. Particularly, in some embodiments, compositions of this invention should be formulated so that a dosage of between 0.01-100 mg/kg body weight/day of a lasso peptide can be administered.

### 5.5 Methods of Using the Lasso Peptides and Compositions Thereof

In a second aspect of the present disclosure, provided herein are methods of managing, preventing, and/or treating an endothelin B receptor (ETBR)-mediated proliferative disease in a subject, where the method comprising administering to the subject an effective amount of a lasso peptide as disclosed herein. In some embodiments of the present method, the lasso peptide comprises an amino acid sequence selected from SEQ ID NOS:1-17 and 42-56. In some embodiments of the present method, the lasso peptide consists essentially of an amino acid sequence selected from SEQ ID NOS:1-17 and 42-56. In some embodiments of the present method, the lasso peptide consists of an amino acid sequence selected from SEQ ID NOS:1-17 and 42-56. In some embodiments of the present method, the lasso peptide comprises an amino acid sequence selected from SEQ ID NOS:1-17 and 42-56, and possesses the lariat conformation through G1-D9 cyclization. In some embodiments of the present method, the lasso peptide consists essentially of an amino acid sequence selected from SEQ ID NOS:1-17 and 42-56, and possesses the lariat conformation through G1-D9 cyclization. In some embodiments of the present method, the lasso peptide consists of an amino acid sequence selected from SEQ ID NOS:1-17 and 42-56, and possesses the lariat conformation through G1-D9 cyclization.

In some embodiments, the subject being treated expresses endothelin B receptor (ETBR), In some embodiments, the subject expresses ETBR in endothelial cells in the microenvironment of the neoplastic cells produced by the proliferative disease being treated. In some embodiments, the subject expresses ETBR in endothelial cells of vasculature in the microenvironment of the neoplastic cells produced by the proliferative disease being treated. In some embodiments, the subject expresses ETBR in the neoplastic cells produced by the proliferative disease being treated. In some embodiments, the ETBR expressed by the subject being treated is ETBR1. In some embodiments, the ETBR expressed by the subject being treated is ETBR2. In some embodiments, the ETBR expressed by the subject being treated is ETBR1 and ETBR2.

In some embodiments, the proliferative disease being treated is cancer. In specific embodiments, the cancer is selected from breast cancer, pancreatic cancer (e.g., pancreatic adenocarcinoma), hepatocellular carcinoma, prostate cancer, ovarian cancer, gastric cancer, glioblastoma, melanoma, cancer of the head and neck, colorectal cancer, bladder cancer, vulvar cancer, esophageal squamous cell carcinoma, renal cancer (e.g., clear-cell renal cell carcinoma), cervical cancer, salivary gland carcinoma, lung cancer (e.g., non-small cell lung cancer and small-cell lung cancer), multiple myeloma, or Kaposi's sarcoma. In particular embodiments, the proliferative disease being treated is melanoma. In particular embodiments, the proliferative disease being treated is ovarian cancer.

In specific embodiments, the cancer is melanoma. In specific embodiments, the cancer is esophageal squamous cell carcinoma. In specific embodiments, the cancer is breast cancer. In specific embodiments, the cancer is glioblastoma. In specific embodiments, the cancer is oligodendroglioma. In specific embodiments, the cancer is bladder cancer. In specific embodiments, the cancer is head and neck cancer. In specific embodiments, the cancer is vulvar cancer. In specific embodiments, the cancer is cervical cancer. In specific embodiments, the cancer is ovarian cancer. In specific embodiments, the cancer is prostate cancer. In specific embodiments, the cancer is clear-cell renal cell carcinoma. In specific embodiments, the cancer is multiple myeloma. In specific embodiments, the cancer is pancreatic adenocarcinoma. In specific embodiments, the cancer is pancreatic Kaposi's sarcoma. In specific embodiments, the cancer is colorectal cancer. In specific embodiments, the cancer is lung cancer.

In some embodiments, the administration of the lasso peptide to the subject inhibits or attenuates activity of endothelin B receptor (ETBR) expressed by the subject, thereby treating the ETBR-mediated proliferative disease. In some embodiments, the administration of the lasso peptides selectively inhibits or attenuates ETBR1 over ETBR2. In some embodiments, the administration of the lasso peptides selectively inhibits or attenuates ETBR2 over ETBR1.

In some embodiments, administration of the lasso peptide to the subject inhibits or attenuates at least one ETBR-mediated signaling pathways. In particular embodiments, the inhibition of ETBR-mediated signaling pathway is measured by (i) inhibition of release of relaxing factors; (ii) upregulation of intercellular adhesion molecule-1 (ICS M-1) expression and clustering; (iii) increasing in migration of intraepithelial tumor infiltrating leukocytes (TILs) into the microenvironment of the neoplastic cells; (iv) inhibition of angiogenesis in the microenvironment of neoplastic cells; (v) inhibition on growth and/or metastasis of neoplastic cells; (vi) increasing in apoptosis of neoplastic cells; or any combination of (i) to (vi). In particular embodiments, the relaxing factors are selected from nitric oxide, prostacyclin, and endothelium-derived hyperpolarizing factor, Ca²⁺, protein kinase C, mitogen-activated protein kinase, or any combination thereof. In specific embodiments, the TILs comprises neutrophils, T cells, B cells, NK cells, monocytes or a combination thereof. In specific embodiments, the monocytes comprise macrophages and/or dendritic cells. In some embodiments, the any of the above activities (i) to (vi) is inhibited at least 1%, 2%, 5%, 10%, 20%, 25%, 50%, 75%, 90%, 95%, 99% or 100%.

In some embodiments, administration of the lasso peptide to the subject downregulates ETBR expression in the subject, thereby treating the ETBR-mediated proliferative disease. In particular embodiments, administration of the lasso peptide to the subject downregulates ETBR expression in endothelial cells in the microenvironment of the neoplastic cells in the subject, thereby treating the ETBR-mediated proliferative disease. In particular embodiments, administration of the lasso peptide to the subject downregulates ETBR expression in endothelial cells of vasculature in the microenvironment of the neoplastic cells in the subject, thereby treating the ETBR-mediated proliferative disease. In particular embodiments, administration of the lasso peptide to the subject downregulates ETBR expression on the surface of neoplastic cells produced by the proliferative disease in the subject, thereby treating the ETBR-mediated proliferative disease. In some embodiments, administration of the lasso peptides to the subject downregulates ETBR expression in the subject by at least 1%, 2%, 5%, 10%, 20%, 25%, 50%, 75%, 90%, 95%, 99% or 100%.

In some embodiments, following administration of the lasso peptides to the subject, the inhibition of at least one ETBR-mediated signaling pathway occurs simultaneously as the downregulation of ETBR expression. In some embodiments, the inhibition of at least one ETBR-mediated signaling pathway occurs before the downregulation of ETBR expression. In some embodiments, the inhibition of at least one ETBR-mediated signaling pathway occurs after the downregulation of ETBR expression. In some embodiments, downregulation of ETBR expression occurs about 1 hour after the inhibition of the at least one ETBR-mediated signaling pathway. In some embodiments, downregulation of ETBR expression occurs about 2 hour after the inhibition of the at least one ETBR-mediated signaling pathway. In some embodiments, downregulation of ETBR expression occurs about 3 hour after the inhibition of the at least one ETBR-mediated signaling pathway. In some embodiments, downregulation of ETBR expression occurs about 4 hour after the inhibition of the at least one ETBR-mediated signaling pathway. In some embodiments, downregulation of ETBR expression occurs about 5 hour after the inhibition of the at least one ETBR-mediated signaling pathway. In some embodiments, downregulation of ETBR expression occurs about 10 hour after the inhibition of the at least one ETBR-mediated signaling pathway.

In some embodiments, the method further comprises administrating to the subject at least one second therapeutic agent for managing, preventing or treating the proliferative disease, where the second therapeutic agent is not a lasso peptide. In some embodiments, the at least second therapeutic agent is co-administered with one or more of the lasso peptides disclosed herein to the subject either simultaneously or sequentially. In specific embodiments, the second therapeutic agent and the lasso peptide are formulated in a single dosage unit for simultaneous administration. In other embodiments, the second therapeutic agent and the lasso peptide are formulated separately for sequential administration. According to the present disclosure, the at least one second therapeutic agent can be administered before or after administration of the lasso peptides. In specific embodiments where the lasso peptides and the second therapeutic agent are administered sequentially, the time gap between their administration can be at least 1 hour, at least 6 hours, at least 12 hours, at least 24 hours, at least 3 days, at least 1 week, at least 2 weeks, at least 1 months, at least 3 months, at least 6 months or at least 1 year. In some embodiments, the subject has been treated with the second therapeutic agent but is found to be non-responsive to the prior treatment, and the subject is then treated with the lasso peptides of the present disclosure.

In some embodiments, the lasso peptide provided herein is co-administered with a chemotherapy. In certain embodiments, the chemotherapeutic agent comprises one or more of cyclophosphamide, thiotepa, mechlorethamine (chlormethine/mustine), uramustine, melphalan, chlorambucil, ifosfamide, chlornaphazine, cholophosphamide, estramustine, novembichin, phenesterine, prednimustine, trofosfamide, uracil mustard, bendamustine, busulfan, improsulfan, piposulfan, carmustine, lomustine, chlorozotocin, fotemustine, nimustine, ranimustine, streptozucin, cisplatin, carboplatin, nedaplatin, oxaliplatin, satraplatin, triplatin tetranitrate, procarbazine, altretamine, dacarbazine, mitozolomide, temozolomide, paclitaxel, docetaxel, vinblastine, vincristine, vinorelbine, cabazitaxel, dactinomycin (actinomycin D), calicheamicin, dynemicin, amsacrine, doxarubicin, daunorubicin, epirubicin, mitoxantrone, idarubicin, pirarubicin, benzodopa, carboquone, meturedopa, uredopa, altretamine, triethylenemelamine, trietylenephosphoramide, triethiylenethiophosphoramide, trimethylolomelamine, bullatacin, bullatacinone, camptothecin, topotecan, bryostatin, callystatin, CC-1065, adozelesin, carzelesin, bizelesin, cryptophycin, dolastatin, duocarmycin, KW-2189, CB1-TM1, eleutherobin, pancratistatin, sarcodictyin, spongistatin, clodronate, esperamicin, neocarzinostatin chromophore, aclacinomysin, anthramycin, azaserine, bleomycin, cactinomycin, carabicin, carminomycin, carzinophilin, chromomycinis, detorubicin, 6-diazo-5-oxo-L-norleucine, esorubicin, idarubicin, marcellomycin, mitomycin, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, mitotane, trilostane, frolinic acid, aceglatone, aldophosphamide glycoside, aminolevulinic acid, eniluracil, bestrabucil, bisantrene, edatraxate, defofamine, demecolcine, diaziquone, elformithine, elliptinium acetate, etoglucid, gallium nitrate, hydroxyurea, lentinan, lonidainine, maytansine, ansamitocins, mitoguazone, mopidanmol, nitraerine, pentostatin, phenamet, pirarubicin, losoxantrone, podophyllinic acid, 2-ethylhydrazide, PSK polysaccharide complex, razoxane, rhizoxin, sizofiran, spirogermanium, tenuazonic acid, triaziquone, 2,2′,2″-trichlorotriethylamine; T-2 toxin, verracurin A, roridin A and anguidine, urethan, vindesine, mannomustine, mitobronitol, mitolactol, pipobroman, gacytosine, arabinoside (“Ara-C”), etoposide (VP-16), vinorelbine, novantrone, teniposide, edatrexate, aminopterin, xeloda, ibandronate, irinotecan (e.g., CPT-11), topoisomerase inhibitor RFS 2000, difluorometlhylornithine (DMFO), retinoic acid, capecitabine, plicomycin, gemcitabine, navelbine, transplatinum, and pharmaceutically acceptable salts, acids, or derivatives of any of the above. In specific embodiments, the chemotherapeutic agent comprises cyclophosphamide.

In some embodiments, the lasso peptide provided herein is co-administered with an immunotherapy. In certain embodiments, the immunotherapy comprises an immune checkpoint modulator that inhibits, decreases or interferes with the activity of a negative checkpoint regulator. In certain embodiments, the negative checkpoint regulator is selected from Cytotoxic T-lymphocyte antigen-4 (CTLA-4), CD40, CD47, CD80, CD86, Programmed cell death 1 (PD-1), Programmed cell death ligand 1 (PD-L1), Programmed cell death ligand 2 (PD-L2), Lymphocyte activation gene-3 (LAG-3; also known as CD223), Galectin-3, B and T lymphocyte attenuator (BTLA), T-cell membrane protein 3 (TIM3), Galectin-9 (GALS), B7-H1, B7-H3, B7-H4, T-Cell immunoreceptor with Ig and ITIM domains (TIGIT/Vstm3/WUCAM/VSIG9), V-domain Ig suppressor of T-Cell activation (VISTA), Glucocorticoid-induced tumor necrosis factor receptor-related (GITR) protein, Herpes Virus Entry Mediator (HVEM), OX40, CD27, CD28, CD137. CGEN-15001T, CGEN-15022, CGEN-15027, CGEN-15049, CGEN-15052, and CGEN-15092. In certain embodiments, the immune checkpoint inhibitor is an anti-PD-1 antibody. In certain embodiments, the immune checkpoint inhibitor is an anti-PD-L1 antibody.

In some embodiments, the lasso peptide provided herein is co-administered with a cancer vaccine. In certain embodiments, the cancer vaccine is selected from sipuleucel-T vaccine, Bacillus Calmette-Guérin vaccine, LLO-E7 DNA vaccine, and T-VEC (Imlygic®).

### 6. EXAMPLES

Examples related to the present invention are described below. In most cases, alternative techniques can be used. The examples are intended to be illustrative and are not limiting or restrictive to the scope of the invention. For example, where lasso peptides or lasso peptide analogs are prepared following a protocol of a Scheme, it is understood that conditions may vary, for example, any of the solvents, reaction times, reagents, temperatures, supplements, work up conditions, or other reaction parameters may be varied.

### General Methods

Reagents used for molecular biology experiments are purchased from New England BioLabs (Ipswich, Mass.), Thermo Fisher Scientific (Waltham, Mass.), or Gold Biotechnology Inc. (St. Louis, Mo.). Other chemicals are purchased from Sigma-Aldrich (St. Louis, Mo.). Escherichia coli DH5α and BL21 (DE3) strains are used for plasmid maintenance and extract production, respectively. Matrix-assisted laser desorption time of flight mass spectrometry (MALDI-TOF-MS) analysis is performed using a Bruker UltrafleXtreme mass spectrometer in reflector positive mode. Electrospray ionization (ESI)-MS/MS analyses are performed using a ThermoFisher Scientific Orbitrap Fusion ESI-MS using an Advion TriVersa Nanomate 100. All molecular biology and cell-free biosynthesis reactions are conducted using standard plates, vial, and flasks typically employed when working with biological molecules such as DNA, RNA and proteins. LC-MS/MS analyses (including Hi-Res analysis) are performed on an Agilent 6530 Accurate-Mass Q-TOF MS equipped with a dual electrospray ionization source and an Agilent 1260 LC system with diode array detector. MS and UV data are analyzed with Agilent MassHunter Qualitative Analysis version B.05.00. Preparative HPLC is carried out using an Agilent 218 purification system (ChemStation software, Agilent) equipped with a ProStar 410 automatic injector, Agilent ProStar UV-Vis Dual Wavelength Detector, a 440-LC fraction collector and preparative HPLC column indicated below. Semi-preparative HPLC purifications are performed on an Agilent 1260 Series Instrument with a multiple wavelength detector and Phenomenex Luna 5 μm C8(2) 250×100 mm semi preparative column. NMR data are acquired using a 600 MHz Bruker Avance III spectrometer with a 1.7 mm cryoprobe. All signals are reported in ppm with the internal DMSO-d6 signal at 2.50 ppm (¹H-NMR) or 39.52 ppm (¹³C-NMR). 1D data is reported as s=singlet, d=doublet, t=triplet, q=quadruplet, m=multiplet or unresolved, br=broad signal, coupling constant(s) in Hz.

For cell culture and fermentation experiments, media used is either M9 minimal medium [17.1 g/L Na2HPO4.12 H2O, 3 g/L KH2PO4, 0.5 g/L NaCl, 1 g/L NH4Cl, 1 mL/L MgSO4 solution (2 M), 0.2 mL/L CaCl2 solution (0.5 M), pH 7.0; after autoclaving, 10 mL/L sterilized glucose solution (40% w/v), 10 mL/L trace metals, and 10 mL/L standard vitamin mix—for trace metals solution, 27 g/L of FeCl₃.6H₂O, 2 g/L of ZnCl₂.4H₂O, 2 g/L of CaCl₂.6H₂O, 2 g/L of Na₂MoO₄.2H₂O, 1.9 g/L of CuSO₄.5H₂O, 0.5 g/L of H₃BO₃, and 100 ml of concentrated HCl; and for vitamin solution, 0.42 g/L of riboflavin, 5.4 g/L of pantothenic acid, 6 g/L of niacin, 1.4 g/L of pyridoxine, 0.06 g/L of biotin, and 0.04 g/L of folic acid]], Luria-Bertani (LB) medium [10 g/L casein peptone, 5 g/L yeast extract, 10 g/L NaCl, pH 7.0], or terrific broth (TB) medium [12 g/L casein peptone, 24 g/L yeast extract, 2.2 g/L KH2PO4, 9.4 g/L K2HPO4, 4 mL/L glycerol, pH 7.0.

To prepare cell extracts, E. coli BL21 Star™ (DE3) cells are grown in the minimum medium containing MM9 salts (13 g/L), calcium chloride (0.1 mM), magnesium sulfate (2 mM), trace elements (2 mM) and glucose (10 g/L), in a 10 L bioreactor (Satorius) to the mid-log growth phase. The grown cells are then harvested and pelleted. The crude cell extracts are prepared as described in Kay, J., et al., Met. Eng., 2015, 32, 133-142 and Sun, Z. Z., J. Vis. Exp. 2013, 79, e50762, doi:10.3791/50762. For calibration of additional magnesium, potassium and DTT levels, a green fluorescence protein (GFP) reporter is used to determine the additional amount of Mg-glutamate, K-glutamate, and DTT that are subsequently added to each batch of the crude cell extracts to prepare the optimized cell extracts for optimal transcription-translation activities. Prior to cell-free biosynthesis of lasso peptide, the optimized cell extracts are pre-mixed with buffer that contains ATP, GTP, TTP, CTP, amino acids, t-RNA, magnesium glutamate, potassium glutamate, potassium phosphate, and other salts, NAD+, NADPH, glucose, 500 uM IPTG and 3 mM DTT to achieve a desirable reaction volume. An exemplary cell extract comprises the ingredients, and optionally with the amounts, as set forth in the following Table 2.

TABLE 2

| Ingredients | Concentration | |
|---|---|---|
| E. coli BL21 Star ™ | 33% v/v | |
| (DE3) extracts | (10 mg/ml of protein or higher) | |
| Amino Acids | 1.5 mM each (Leucine, 1.25 mM) | |
| HEPES | 50 | mM |
| ATP | 1.5 | mM |
| GTP | 1.5 | mM |
| CTP & UTP | 0.9 | mM |
| tRNA | 0.2 | mg/mL |
| CoA | 0.26 | mM |
| NAD+ | 0.33 | mM |
| cAMP | 0.75 | mM |
| Folinic acid | 0.068 | mM |
| spermidine | 1 | mM |
| pEG-8000 | 2% | |
| magnesium glutamate | 4-12 | mM |
| potassium glutamate | 8-160 | mM |
| potassium phosphate | 1-10 | mM |
| DTT | 0-5 | mM |
| NADPH | 1 | mM |
| maltodextrin | 35 | mM |
| IPTG (optional) | 0.5 | mM |
| pyruvate | 30 | mM |
| NADH | 1 | mM |

Affinity chromatography procedures are carried out according to the manufacturers' recommendations to isolate lasso peptides fused to an affinity tag; for examples, Strep-tag® II based affinity purification (Strep-Tactin® resin, IBA Lifesciences), His-tag-based affinity purification (Ni-NTA resin, ThermoFisher), maltose-binding protein-based affinity purification (amylose resin, New England BioLabs). The sample of lasso peptides fused to an affinity tag is lyophilized and resuspended in a binding buffer with respect to its affinity tag according to the manufacturer's recommendation. The resuspended lasso peptide sample is directly applied to an immobilized matrix corresponding to its fused affinity tag (Tactin for Strep-tag® II, Ni-NTA for His-tag, or amylose resin for maltose binding protein) and incubated at 4° C. for an hour. The matrix is then washed with at least 40× volume of washing buffer and eluted with three successive 1× volume of elution buffer containing 2.5 mM desthiobiotin for Strep-Tactin® resin, 250 mM imidizole for Ni-NTA resin or 10 mM maltose for amylose resin. The eluted fractions are analyzed on a gradient (10-20%) Tris-Tricine SDS-PAGE gel (Mini-PROTEAN, BioRad) and then stained with Coomassie brilliant blue.

The purity of eluted lasso peptide is examined by LC-MSMS on an Agilent 6530 Accurate-Mass Q-TOF mass spectrometer. Where possible, MSMS fragmentation is used to further characterize lasso peptides based on the rule described in Fouque, K. J. D, et al., Analyst, 2018, 143, 1157-1170. If impurities are observed in chromatographic spectra, preparative chromatography is performed to further enrich the purity of lasso peptides.

Analytical LCMS Analytical Method:
Column: Phenomenex Kinetex 2.6μ XB-C18 100 A, 150×4.6 mm column.
Flow rate: 0.7 mL/min
Temperature: RT
Mobile Phase A: 0.1% formic acid in water
Mobile Phase B: acetonitrile or methanol
Injection amount: 2
HPLC Gradient: 10% B for 3.0 min, then 10 to 100% B over 20 minutes follow by 100% B for 3 min. 4 minute post run equilibration time

Preparative HPLC is carried out using an Agilent 218 purification system (ChemStation software, Agilent) equipped with a ProStar 410 automatic injector, Agilent ProStar UV-Vis Dual Wavelength Detector, a 440-LC fraction collector. Fractions containing lasso peptides are identified using the LCMS method described above, or by direct injection (bypassing the LC column in the above method) prior to combining and lyophilizing. Analytical LC/MS (see method above) is then performed on the combined and concentrated lasso peptides.

Preparative HPLC Method:
Column: Phenomenex Luna® preparative column 5 μM, C18(2) 100 Å 100×21.2 mm
Flow rate: 15 mL/min
Temperature: RT
Mobile Phases: water, MeOH, acetonitrile, formic acid, in different percentages and used as gradients
Injection amount: varies

If necessary, semi-preparative HPLC purifications are performed on an Agilent 1260 Series Instrument with a multiple wavelength detector
Semipreparative HPLC Method:
Column: Phenomenex Luna® 5 μm C18(2) 250×100 mm
Flow rate: 4 mL/min
Temperature: RT
Mobile Phases: water, MeOH, acetonitrile, formic acid, in different percentages and used as gradients
Injection amount: varies

Monoisotopic masses are extrapolated from the lasso peptide charge envelop [(M+H)¹⁺, (M+2H)²⁺, (M+3H)³⁺] in the m/z 500-3,200 range using a Agilent 6530 Accurate-Mass Q-TOF MS equipped with a dual electrospray ionization source and an Agilent 1260 LC system using an internal reference (see analytical procedure described above). Both MS and MS/MS analyses are performed in positive-ion mode.

NMR samples are dissolved in DMSO-d6 (Cambridge Isotope Lab-oratories). All NMR experiments are run on a 600 MHz Bruker Avance III spectrometer with a 1.7 mm cryoprobe. All signals are reported in ppm with the internal DMSO-d6 signal at 2.50 ppm (¹H-NMR) or 39.52 ppm (¹³C-NMR). Where applicable, structural characterization of lasso peptide follow the methods described in the literatures listed below:
1. Knappe et al., J. Am. Chem. Soc., 2008, 130 (34), 11446-11454
2. Maksimov et al., PNAS, 2012, 109 (38), 15223-15228
3. Tietz et al., Nature Chem. Bio., 2017, 13, 470-478
4. Zheng and Price, Prog Nucl Magn Reson Spectrosc, 2010, 56 (3), 267-288
5. Marion et al., J Magn Reson, 1989, 85 (2), 393-399
6. Davis et al., J Magn Reson, 1991, 94 (3), 637-644
7. Rucker and Shaka, Mol Phys, 1989, 68 (2), 509-517
8. Hwang and Shaka, J Magn Reson A, 1995, 112 (2), 275-27

Sequences used in the following examples are summarized in following Table 3.

TABLE 3

| | A.A. Sequence | Encoding Nucleotide Sequence | GenBank |
|---|---|---|---|
| Peptide | (SEQ ID NO:) | (SEQ ID NO:) | Accession |
| Precursor | MTEQSEQTPAEYIPPMLVEV | | |
| Peptide (A) for | GEFTEDTLGNWHGTAPDWF | | |
| Lasso 1 | FNYYW (18) | | |
| Precursor | MTEQSEQTPAEYIPPMLVEV | ATGACCGAGCAGTCCGAGCAGACACCCGCGGAATAC | RES-701-3_A |
| Peptide (A) for | GEFTEDTLGNWHGTSPDWF | ATTCCGCCGATGCTCGTCGAGGTCGGCGAGTTCACCG | WP_106430389.1 |
| Lasso 3 | FNYYW (20) | AAGACACGTTGGGCAACTGGCACGGCACATCGCCCG | |
| | | ACTGGTTCTTCAACTACTACTGG (38) | |
| Lasso | MSQSMALDQRARLPLARRL | ATGAGCCAGTCCATGGCACTGGACCAGCGGGCCCGG | RES-701-3_B |
| peptidase (B) | LPLLAVGAARPLARLKPARL | CTCCCGCTTGCGCGCCGTCTGCTGCCGCTGCTGGCCG | WP_040898778.1 |
| | RAVLEFARRGAAPAGAGQA | TCGGCGCCGCCCGGCCGCTCGCCCGCCTCAAGCCCGC | |
| | QRAREQVVSVSLRCAGQNC | GCGGCTGCGCGCCGTGCTCGAATTCGCCCGCCGTGGT | |
| | LQRSLATVLLCRARGVWPT | GCGGCGCCGGCGGGTGCCGGGCAGGCGCAGCGGGCG | |
| | WCTGVRTHPFAAHAWVEAE | CGCGAGCAGGTCGTCTCGGTGAGCCTGCGCTGTGCGG | |
| | GRLIGEPHPEGYYKPLLTVPP | GGCAGAACTGTCTGCAGCGCTCGCTCGCCACCGTGCT | |
| | RTGAGAGDR (35) | GCTGTGCCGGGCCCGCGGGGTGTGGCCGACCTGGTG | |
| | | CACCGGCGTACGGACGCACCCGTTCGCCGCCCACGCC | |
| | | TGGGTTGAGGCCGAGGGCCGGCTGATCGGCGAACCG | |
| | | CACCCCGAGGGCTATTACAAGCCGCTGCTGACCGTCC | |
| | | CGCCCCGCACCGGAGCCGGCGCCGGGGACCGC (39) | |
| Lasso cyclase | MAKLSAGFVVLPDTADGAEI | ATGGCGAAGTTGAGTGCTGGGTTTGTGGTCCTGCCGG | RES-701-3_C |
| (C) | RALVPFAPARVIAHASGRPW | ACACCGCCGACGGTGCGGAGATCCGGGCGCTCGTAC | WP_006604202.1 |
| | LVGEWSQGQVRVASAGPVR | CGTTCGCGCCTGCCCGAGTGATCGCGCACGCCTCCGG | |
| | LAVSGTCPVTDDGLARIAAR | GCGGCCGTGGCTGGTCGGCGAGTGGTCTCAGGGTCA | |
| | ISRLSEVDRAVPALLGSCHIV | GGTGAGGGTGGCTTCGGCGGGTCCGGTACGTCTGGCC | |
| | ASVDGHVRFQGSASGLRRVF | GTGAGCGGCACGTGTCCGGTCACCGACGACGGGCTG | |
| | HTRVNGVRVAADRSDVLAA | GCGCGGATTGCCGCCCGTATCTCCCGGCTGTCCGAGG | |
| | MTGAGVDEETLALHVACGL | TGGACCGGGCCGTTCCCGCGCTGCTGGGGTCCTGCCA | |
| | QVPFPANARSAWSGISTLAP | CATCGTGGCATCGGTGGACGGGCACGTCCGGTTCCAG | |
| | ENCLLWEGDRDRETVWWRP | GGCAGCGCCTCCGGGCTGCGTCGCGTGTTCCACACCC | |
| | PEPDRSLREGTAAVRDTLAA | GGGTGAACGGGGTGCGGGTGGCCGCGGACCGGTCCG | |
| | VVGRQAPVEGRLSADLSGG | ATGTGCTCGCGGCGATGACCGGTGCCGGGGTGGACG | |
| | LDSTSLCFLAARHTPELLTFR | AGGAGACGCTGGCACTGCACGTCGCATGCGGTTTGC | |
| | WGEADAGNDDAVYAGQAA | AGGTGCCCTTTCCGGCCAACGCGCGCAGCGCGTGGTC | |
| | RLLDRAEHLVVPQHELPDIF | CGGCATCAGCACGCTCGCGCCGGAGAACTGTCTGCTG | |
| | ADPALAVSAEEPLSLTRATA | TGGGAGGGCGACCGGGACCGGGAAACGGTGTGGTGG | |
| | RIRRGARLLADRGSRRHLAG | CGTCCGCCCGAGCCGGACCGGTCGTTGCGGGAGGGG | |
| | HGGDELFSPMPSYLHQLMR | ACGGCAGCGGTACGGGACACCCTGGCTGCCGTAGTG | |
| | RHPVTALKHVRAHCALKRW | GGCCGACAAGCGCCCGTCGAAGGCCGGTTGAGTGCG | |
| | PLKATLAALAQPGSLPAWW | GATCTGTCCGGCGGGCTGGATTCGACGTCGCTGTGTT | |
| | REQAGLLTEPRPPLRRPALG | TTCTGGCGGCCCGGCACACACCGGAGCTGCTGACCTT | |
| | WGLGPVRAAPWMTPEGVEL | CCGGTGGGGCGAGGCCGACGCGGGCAATGACGACGC | |
| | AREALRRTAEWAQPLAEDL | GGTCTACGCGGGGCAGGCGGCGCGGCTGCTCGACCG | |
| | ATHTMVLMIRSNTASYRLLA | TGCCGAGCACCTCGTCGTGCCGCAGCACGAACTCCCC | |
| | RLYAEAGVELDMPYLDDAV | GACATCTTCGCCGACCCGGCCCTCGCGGTGTCCGCCG | |
| | IDAVLRVPAHAHAGPWRYK | AGGAGCCGCTGTCGCTGACGCGCGCGACGGCCCGGA | |
| | PLLADAMHGIVPDGIRARST | TCCGCCGGGGCGCCCGACTGCTCGCGGACCGCGGCTC | |
| | KGEFGEDIRRGLRRNLPAILD | CCGTCGGCATCTGGCCGGGCACGGCGGCGACGAGCT | |
| | LFADSELAARGLIDTGALRL | GTTCTCCCCGATGCCCTCCTATCTGCATCAGTTGATGC | |
| | RLGGPQPDNTTAQALESLLG | GCCGTCATCCGGTGACGGCGCTCAAGCACGTACGGG | |
| | CETWLRTTSGPGTIPRAGDG | CGCACTGCGCGCTGAAGCGCTGGCCGCTGAAGGCGA | |
| | TVPSEV (36) | CGCTTGCCGCGCTCGCGCAGCCGGGAAGTCTCCCCGC | |
| | | GTGGTGGCGGGAGCAGGCCGGGCTGCTGACCGAGCC | |
| | | CCGGCCGCCGCTGCGGCGCCCGGCGCTGGGCTGGGG | |
| | | TCTGGGACCCGTACGTGCGGCGCCCTGGATGACCCCC | |
| | | GAGGGCGTCGAGCTGGCGCGGGAGGCGCTGCGCCGG | |
| | | ACCGCCGAGTGGGCGCAGCCGCTCGCCGAGGACCTC | |
| | | GCCACCCACACGATGGTGCTCATGATCCGTTCGAACA | |
| | | CGGCCAGTTACCGGTTGCTGGCGCGGCTGTACGCCGA | |
| | | GGCCGGTGTGGAGCTGGACATGCCGTATCTGGACGA | |
| | | CGCCGTGATCGACGCGGTGCTCCGGGTACCGGCGCA | |
| | | CGCGCACGCCGGACCGTGGCGGTACAAGCCCCTGCT | |
| | | GGCCGATGCGATGCACGGCATCGTGCCGGACGGCAT | |
| | | CCGGGCGCGGTCCACCAAGGGCGAGTTCGGTGAGGA | |
| | | CATCAGAAGGGGGCTGCGTCGCAACCTGCCCGCCAT | |
| | | CCTGGACCTCTTCGCCGATTCCGAGCTGGCGGCCCGC | |
| | | GGTCTGATCGACACCGGCGCGCTCCGGCTGCGGCTGG | |
| | | GCGGGCCGCAGCCGGACAACACCACCGCCCAGGCGC | |
| | | TGGAGAGCCTGCTCGGCTGCGAGACCTGGCTGCGCA | |
| | | CCACGTCCGGCCCCGGCACCATCCCCCGTGCCGGCGA | |
| | | CGGCACCGTTCCCTCGGAGGTC (40) | |
| Lasso RRE (E) | MRLHPDVSMTDTDDGTVLL | ATGCGGCTCCATCCCGACGTCTCCATGACGGACACCG | RES-701-3_E |
| | HQRTGRYWQLNVTGSRVLH | ACGACGGCACGGTGCTGCTGCATCAGCGCACCGGCC | WP_106430390.1 |
| | RLLDGDTPETIADGLAAAHG | GCTACTGGCAGCTCAACGTCACCGGCAGCCGCGTGCT | |
| | IDPQRARQDVGAVIEQLRTA | GCACCGGCTGCTGGACGGGGACACCCCCGAGACCAT | |
| | ELTVAS (37) | CGCCGACGGCCTCGCCGCCGCGCACGGCATCGACCC | |
| | | GCAGCGGGCCCGGCAGGATGTCGGCGCCGTGATCGA | |
| | | GCAACTGCGCACCGCCGAGCTGACGGTGGCCTCA (41) | |

### Example 1. Cell-Free Biosynthesis of Lasso Peptide (Lasso 3)

Synthesis of lasso peptide Lasso 3 with the sequence of GNWHGTSPDWFFNYYW (SEQ ID NO: 3) by adding the individually cloned genes for the lasso precursor peptide MTEQSEQTPAEYIPPMLVEVGEFTEDTLGNWHGTSPDWFFNYYW (SEQ ID NO:20), peptidase (SEQ ID NO:35), cyclase (SEQ ID NO:36), and RRE (SEQ ID NO:37) to cell extract, where the N-terminal amine group of a glycine (G) residue at the first position is cyclized with the side-chain carboxylic acid group of an aspartic acid (D) residue at the ninth position.

DNA encoding sequences for the lasso precursor peptide, peptidase, cyclase, and RRE, identified in Streptomyces auratus AGR0001 genomic data using RODEO, are codon-optimized for use in E. coli and synthesized (Thermo Fisher, Waltham, Mass.). Each linear gene is individually cloned into a pZE expression vector behind a T7 promoter (Expressys). The resulting plasmids encoding genes for the lasso precursor peptide, peptidase, cyclase, and RRE are used either with or without an N-terminal affinity tag (e.g., maltose-binding protein) or a C-terminal affinity tag (e.g., His-6). Production of lasso peptide Lasso 3 is initiated by adding the plasmids encoding the lasso precursor peptide, peptidase, cyclase, and RRE (15 nM each) to optimized E. coli BL21 Star™ (DE3) cell extracts, which are pre-mixed with buffer that contains ATP, GTP, TTP, CTP, amino acids, t-RNA, magnesium glutamate, potassium glutamate, potassium phosphate, and other salts, NAD+, NADPH, and glucose (Table XI) to achieve a total volume of 400 μL. The cell-free biosynthesis of lasso peptide Lasso 3 is accomplished by incubating the reaction for 18 hours at 20° C. The reaction sample is subsequently diluted in MeOH at 1:1 ratio (v/v) and thoroughly mixed at room temperature for 30 minutes, followed by centrifugation at 14,000 rpm in an Eppendorf benchtop centrifuge to remove precipitated protein. The resulting liquid fraction is subjected to LC/MS analysis on an Agilent 6530 Accurate-Mass Q-TOF MS equipped with a dual electrospray ionization source and an Agilent 1260 LC system with diode array detector for lasso peptide detection. The expected molecular mass of 2058.8 m/z corresponding to the desired lasso peptide GNWHGTSPDWFFNYYW (SEQ ID NO: 3) is observed. The collected lasso peptide sample is further purified by affinity chromatography and/or semi-preparative HPLC, followed by structural characterization by high resolution LC-MS or NMR and biological characterization by screening in endothelin receptor and cancer cell growth assays.

### Example 2. Cell-Based Biosynthesis of Lasso Peptide Lasso 3 Using E. coli

Synthesis of lasso peptide Lasso 3 with primary sequence GNWHGTSPDWFFNYYW (SEQ ID NO: 3) by cloning the genes for the lasso precursor peptide, peptidase, cyclase, and RRE into plasmids that are transformed into E. coli, where the N-terminal amine group of a glycine (G) residue at the first position is cyclized with the side-chain carboxylic acid group of an aspartic acid (D) residue at the ninth position.

DNA encoding sequences corresponding to an open reading frame (ORF) for the lasso precursor peptide, peptidase, cyclase, and RRE, are identified by analyzing Streptomyces auratus AGR0001 genomic data using RODEO and codon-optimized for use in E coli. Each ORF is constructed by PCR from corresponding oligonucleotides and cloned into pCR™-Blunt II-TOPO™ (Thermo, Waltham, Mass.) for sequence verification. Thirty base pairs with homology to the expression vector sequence is added upstream and downstream of each ORF during amplification for scarless cloning into the expression vector by Gibson Assembly™ Cloning Kit (NEB; Ipswich, Mass.). Two expression vectors are used, namely pETDuet-1 (EMD Millipore, Burlington, Mass.) and pACYCDuet-1 (EMD Millipore, Burlington, Mass.). Both vectors are designed such that they have the Lad protein expressed from the backbone, and also have bi-cistronic multi-cloning sites (MCSs) such that the T7 gene 10 promoter precedes a Lac operator site upstream of the RBS site from T7 gene 10 promoter followed by a MCS and the T7 gene 10 terminator, to provide the generalized structure of the two Duet expression vector constructs (T7 promoter-LacO operator-RBS-MCS1-T7 terminator-T7 promoter-LacO operator-RBS-MCS2-T7 terminator). The ORFs are inserted sequentially into the Duet vectors with the vector linearized at the desired location of the MCS after linearization by the appropriate restriction enzyme and Gibson Assembly (Ultra) per the manufacturer's recommended conditions (SGI-DNA, La Jolla, Ca). ORFs corresponding to lasso precursor peptide and lasso cyclase are inserted into MCS1 and MCS2 of pETDuet-1, respectively, while ORFs corresponding to lasso peptidase and RRE are inserted into MCS1 and MCS2 of pACYCDuet-1, respectively. The resulting expression constructs pETDuet-1 and pACYCDuet-1 are each transformed into E. coli DH10B cells and grown overnight in the presence of ampicillin (100 μg/mL) and chloramphenicol (30 μg/mL), respectively, and vectors are isolated, purified, and sequence verified. For expression, the constructs are co-transformed into E. coli BL21 (DE3) cells and selected with ampicillin (100 μg/mL) and chloramphenicol (30 μg/mL). After plating on agar and growth for 24 h, a single, large colony is picked from the plate, inoculated into 5 mL LB broth containing ampicillin (100 μg/mL) and chloramphenicol (30 μg/mL) and shaken overnight 37° C. The 5 mL LB culture is added to M9 minimal medium (0.5 L) in a 2 L baffled flask and cells are grown at 37° C. until an optical density at 600 nm (0D600) of 0.4 is reached, then the temperature is reduced to 20° C. Cultures are induced 1 h later (0D600 of 0.5-0.6) by addition of 100 μL of a 0.5 M stock solution of isopropyl β-D-1-thiogalactopyranoside (IPTG), yielding a final IPTG concentration of 0.1 mM. The culture is shaken for 3 d at 20° C. and then harvested by centrifugation. Cell pellets are extracted by resuspending them in 50 mL of MeOH and shaking overnight at 4° C. Afterward, extracts are spun down, and the clear supernatant is collected and evaporated to dryness under reduced pressure. The dry residue is resuspended in 1 mL of 50% methanol, cleared by centrifugation, and analyzed by high resolution LC-MS. The expected molecular mass of 2058.8 m/z corresponding to the desired lasso peptide GNWHGTSPDWFFNYYW (SEQ ID NO: 3) is observed. Supernatant fractions are pooled and mixed with 6% (wt/vol) Diaion HP-20 resin beads (Sigma-Aldrich, St. Louis, Mo., USA) and left overnight. The resin beads are recovered by filtering the solution through glass wool and then soaking in methanol. The methanol fraction is filtered through filter paper in a vacuum system. The eluent is concentrated at 40° C. at reduced pressure, and the resulting solution is fractionated through an ion exchange column (Strata SAX) (55-μm pore size, 70 A, 500 mg/3 ml) using 10% of the packing weight as the loading fraction. The column is equilibrated with 20 column volumes of water before the sample is loaded and washed with 10 column volumes (30 ml) of different mixtures of methanol-water solutions i to v as follows: (i) 100% water; (ii) 75% water and 25% methanol; (iii) 50% water and 50% methanol; (iv) 25% water and 75% methanol; (iv) 100% methanol; (v) 100% methanol and 0.1% trifluoroacetic acid (TFA). An aliquot of each fraction is concentrated and analyzed by high resolution LC-MS. Fractions containing product are pooled and concentrated and subjected to semipreparative high-performance liquid chromatography (HPLC; Agilent 1260) through a C18 column connected to a binary pump and a photodiode array detector set at 280 nm. Use of gradient elution with water, methanol, and 0.1% formic acid affords the desired lasso peptide which is analyzed by high resolution LC-MS. The expected molecular mass of 2058.8 m/z corresponding to the desired lasso peptide GNWHGTSPDWFFNYYW (SEQ ID NO: 3) is observed.

### Example 3. Cell-Free Biosynthesis of Lasso Peptide Lasso 1

Synthesis of lasso peptide Lasso 1 with primary sequence GNWHGTAPDWFFNYYW (SEQ ID NO: 1) by adding the individually cloned genes for the lasso precursor peptide MTEQSEQTPAEYIPPMLVEVGEFTEDTLGNWHGTAPDWFFNYYW (SEQ ID NO:18), peptidase, cyclase, and RRE to cell extract, where the N-terminal amine group of a glycine (G) residue at the first position is cyclized with the side-chain carboxylic acid group of an aspartic acid (D) residue at the ninth position.

The DNA sequence encoding lasso precursor peptide MTEQSEQTPAEYIPPMLVEVGEFTEDTLGNWHGTAPDWFFNYYW (SEQ ID NO:18) is produced from SEQ ID NO:20 by using the QuikChange Mutagenesis method (Agilent, Santa Clara, Calif., USA) as per manufacturer's instructions using Q5 DNA polymerase (New England BioLabs (Ipswich, Mass.). Lasso peptidase, cyclase, and RRE, identified in Streptomyces auratus AGR0001 genomic data using RODEO, are codon-optimized for use in E coli and synthesized as described in Example 1 (Thermo Fisher, Waltham, Mass.). Each linear gene is individually cloned into a pZE expression vector behind a T7 promoter (Expressys). The resulting plasmids encoding genes for the lasso precursor peptide, peptidase, cyclase, and RRE are used either with or without an N-terminal affinity tag (e.g., maltose-binding protein) or a C-terminal affinity tag (e.g., His-6). Production of lasso peptide Lasso 3 is initiated by adding the plasmids encoding the lasso precursor peptide, peptidase, cyclase, and RRE (15 nM each) to optimized E. coli BL21 Star™ (DE3) cell extracts, which are pre-mixed with buffer that contains ATP, GTP, TTP, CTP, amino acids, t-RNA, magnesium glutamate, potassium glutamate, potassium phosphate, and other salts, NAD+, NADPH, and glucose (Table XI) to achieve a total volume of 400 μL. The cell-free biosynthesis of lasso peptide (Lasso 1) is accomplished by incubating the reaction for 18 hours at 20° C. The reaction sample is subsequently diluted in MeOH at 1:1 ratio (v/v) and thoroughly mixed at room temperature for 30 minutes, followed by centrifugation at 14,000 rpm in an Eppendorf benchtop centrifuge to remove precipitated protein. The resulting liquid fraction is subjected to LC/MS analysis on an Agilent 6530 Accurate-Mass Q-TOF MS equipped with a dual electrospray ionization source and an Agilent 1260 LC system with diode array detector for lasso peptide detection. The molecular mass of 2042.9 m/z corresponding to the desired lasso peptide GNWHGTAPDWFFNYYW (SEQ ID NO: 1) is observed. The collected lasso peptide sample is further purified by affinity chromatography and/or semi-preparative HPLC, followed by structural characterization by high resolution LC-MS or NMR and biological characterization by screening in endothelin receptor and cancer cell growth assays.

### Example 4. Cell-Based Biosynthesis of Lasso Peptide Lasso 1

Synthesis of lasso peptide Lasso 1 with primary sequence GNWHGTAPDWFFNYYW Lasso 1 by cloning the genes for the lasso precursor peptide (SEQ ID NO:18), peptidase, cyclase, and RRE into plasmids that are transformed into E. coli, where the N-terminal amine group of a glycine (G) residue at the first position is cyclized with the side-chain carboxylic acid group of an aspartic acid (D) residue at the ninth position.

The DNA sequence encoding lasso precursor peptide MTEQSEQTPAEYIPPMLVEVGEFTEDTLGNWHGTAPDWFFNYYW (SEQ ID NO:18) is produced from introducing a single amino acid substitute in the precursor peptide having the sequence of MTEQSEQTPAEYIPPMLVEVGEFTEDTLGNWHGTSPDWFFNYYW (SEQ ID NO:20) by using the QuikChange Mutagenesis method (Agilent, Santa Clara, Calif., USA) as per manufacturer's instructions using Q5 DNA polymerase (New England BioLabs (Ipswich, Mass.). DNA encoding sequences corresponding to an open reading frame (ORF) for the lasso precursor peptide, peptidase, cyclase, and RRE, are identified by analyzing Streptomyces auratus AGR0001 genomic data using RODEO. Each ORF is constructed by PCR from corresponding oligonucleotides that are codon-optimized for E. coli and cloned into pCR™-Blunt II-TOPO™ (Thermo, Waltham, Mass.) for sequence verification. Thirty base pairs with homology to the expression vector sequence is added upstream and downstream of each ORF during amplification for scarless cloning into the expression vector. Two expression vectors are used, namely pETDuet-1 (EMD Millipore, Burlington, Mass.) and pACYCDuet-1 (EMD Millipore, Burlington, Mass.). Both vectors are designed such that they have the Lad protein expressed from the backbone, and also have bi-cistronic multi-cloning sites (MCSs) such that the T7 gene 10 promoter precedes a Lac operator site upstream of the RBS site from T7 gene 10 promoter followed by a MCS and the T7 gene 10 terminator, to provide the generalized structure of the two Duet expression vector constructs (T7 promoter-LacO operator-RBS-MCS1-T7 terminator-T7 promoter-LacO operator-RBS-MCS2-T7 terminator). The ORFs are inserted sequentially into the Duet vectors with the vector linearized at the desired location of the MCS after linearization by the appropriate restriction enzyme and Gibson Assembly (Ultra) per the manufacturer's recommended conditions (SGI-DNA, La Jolla, Ca). ORFs corresponding to lasso precursor peptide and lasso cyclase are inserted into MCS1 and MCS2 of pETDuet-1, respectively, while ORFs corresponding to lasso peptidase and RRE are inserted into MCS1 and MCS2 of pACYCDuet-1, respectively. The resulting expression constructs pETDuet-1 and pACYCDuet-1 are each transformed into E. coli DH10B cells and grown overnight in the presence of ampicillin (100 μg/mL) and chloramphenicol (30 μg/mL), respectively, and vectors are isolated, purified, and sequence verified. For expression, the constructs are co-transformed into E. coli BL21 (DE3) cells and selected with ampicillin (100 μg/mL) and chloramphenicol (30 μg/mL). After plating on agar and growth for 24 h, a single, large colony is picked from the plate, inoculated into 5 mL LB broth containing ampicillin (100 μg/mL) and chloramphenicol (30 μg/mL) and shaken overnight 37° C. The 5 mL LB culture is added to M9 minimal medium (0.5 L) in a 2 L baffled flask and cells are grown at 37° C. until an optical density at 600 nm (0D600) of 0.4 is reached, then the temperature is reduced to 20° C. Cultures are induced 1 h later (0D600 of 0.5-0.6) by addition of 100 μL of a 0.5 M stock solution of isopropyl β-D-1-thiogalactopyranoside (IPTG), yielding a final IPTG concentration of 0.1 mM. The culture is shaken for 3 d at 20° C. and then harvested by centrifugation. Cell pellets are extracted by resuspending them in 50 mL of MeOH and shaking overnight at 4° C. Afterward, extracts are spun down, and the clear supernatant is collected and evaporated to dryness under reduced pressure. The dry residue is resuspended in 1 mL of 50% methanol, cleared by centrifugation, and analyzed by high resolution LC-MS. The expected molecular mass of 2058.8 m/z corresponding to the desired lasso peptide GNWHGTSPDWFFNYYW (SEQ ID NO: 3) is observed. Supernatant fractions are pooled and mixed with 6% (wt/vol) Diaion HP-20 resin beads (Sigma-Aldrich, St. Louis, Mo., USA) and left overnight. The resin beads are recovered by filtering the solution through glass wool and then soaking in methanol. The methanol fraction is filtered through filter paper in a vacuum system. The eluent is concentrated at 40° C. at reduced pressure, and the resulting solution is fractionated through an ion exchange column (Strata SAX) (55-μm pore size, 70 A, 500 mg/3 ml) using 10% of the packing weight as the loading fraction. The column is equilibrated with 20 column volumes of water before the sample is loaded and washed with 10 column volumes (30 ml) of different mixtures of methanol-water solutions i to v as follows: (i) 100% water; (ii) 75% water and 25% methanol; (iii) 50% water and 50% methanol; (iv) 25% water and 75% methanol; (iv) 100% methanol; (v) 100% methanol and 0.1% trifluoroacetic acid (TFA). An aliquot of each fraction is concentrated and analyzed by high resolution LC-MS. Fractions containing product are pooled and concentrated and subjected to semipreparative high-performance liquid chromatography (HPLC; Agilent 1260) through a C18 column connected to a binary pump and a photodiode array detector set at 280 nm. Use of gradient elution with water, methanol, and 0.1% formic acid affords the desired lasso peptide which is analyzed by high resolution LC-MS. The expected molecular mass of 2058.8 m/z corresponding to the desired lasso peptide GNWHGTSPDWFFNYYW (SEQ ID NO: 3) is observed.

### Example 5. Comparison of Lasso 1 Inhibition of Endothelin-1 Binding to Endothelin a Receptor (ETAR) and Endothelin B Receptor (ETBR) Expressed on the Surface of CHO Cells

Cell culture. CHO cells are maintained in DMEM medium supplemented with 10% FCS and 2 mM glutamate under a humidified 5% CO2-95% air atmosphere.

Stable expression of ETA or ETB in CHO cells. Cell lines stably expressing ETAR or ETBR (CHO-ETAR and CHO-ETBR, respectively) are obtained using a mammalian expression vector, pME18S-FL3 (GSL Biotech, Chicago, Ill., USA), that carries a cDNA construct encoding human recombinant ETA receptor or ETB receptor. Each expression vector is co-transfected with pSVbse plasmid into CHO cells by lipofection using Lipofectamine 2000 (Thermo Fisher, Carlsbad, Calif., USA) according to the manufacturer's instructions. Cell populations expressing the bsr^{r} gene product are selected in DMEM medium supplemented with 10% FCS, 2 mM glutamate and 0.5 mg/ml blasticidin. Blasticidin-selected clonal cell populations are isolated by colony lifting and confirmed to express either ETAR or ETBR by surface staining with antibodies (ab85163 and ab117529, respectively for ETAR and ETBR; Abcam, Cambridge, Mass.) in combination with fluorescence-activated cell sorting (FACS). Validated cells are maintained in the same selection medium. Binding experiments are conducted with membranes prepared from the stably transfected CHO-ETAR and CHO-ETBR cells.

Preparation of Membranes. CHO cell membranes are prepared by washing ETAR- or ETBR-transfected cells with phosphate buffered saline and centrifugation. The resulting cell pellets are homogenized with a polytron (settings 8 for 30 seconds×2) in 5 volumes of buffer A containing 1 mM NaHCO₃, 5 mM EDTA (pH 8.3), 5 μg/ml Leupeptin, 5 μg/ml Pepstatin A, and 40 μM phenylmethylsulfonyl fluoride. The homogenates are centrifuged at 8,000×g for 10 minutes and supernatants are then centrifuged at 40,000×g for 1 hour at 4° C. The pellets are homogenized in buffer A and recentrifuged at 40,000×g for 1 hour. The resulting pellets are homogenized in buffer A supplemented with 130 mM NaCl, 5 mM Na₂HPO₄, and 1.5 mM KH₂PO₄ and stored at −70° C.

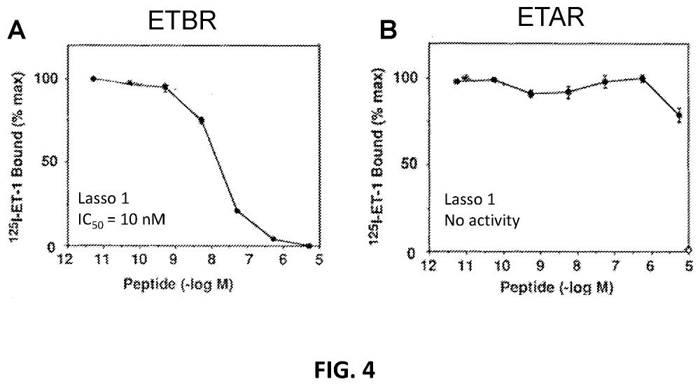

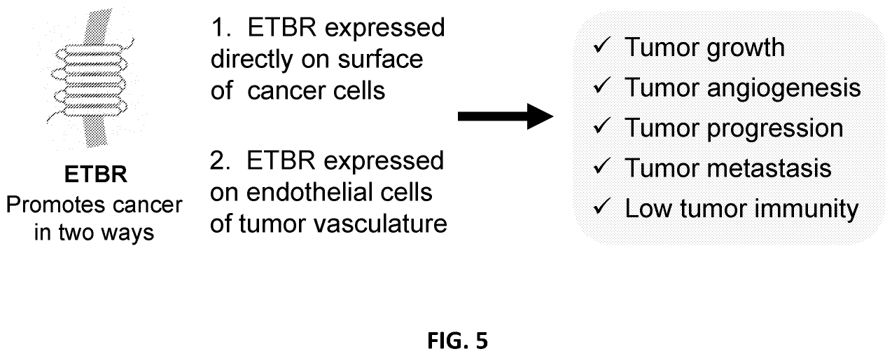

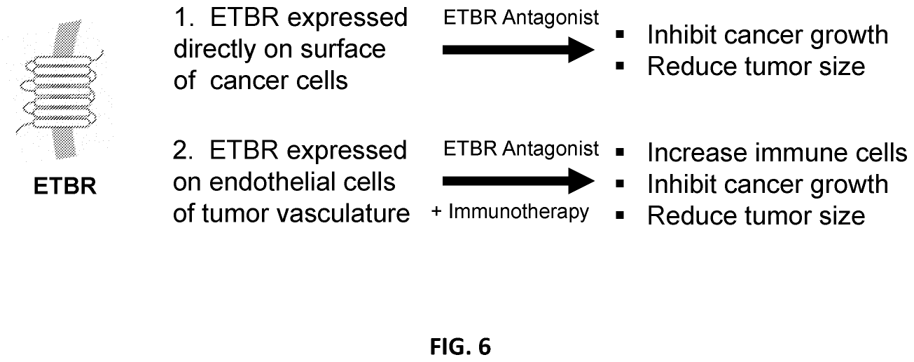

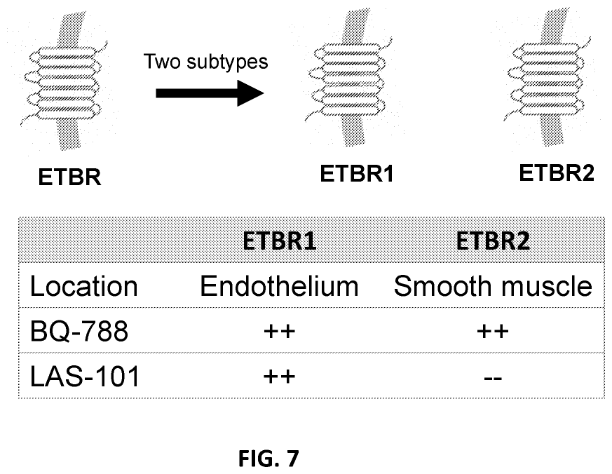

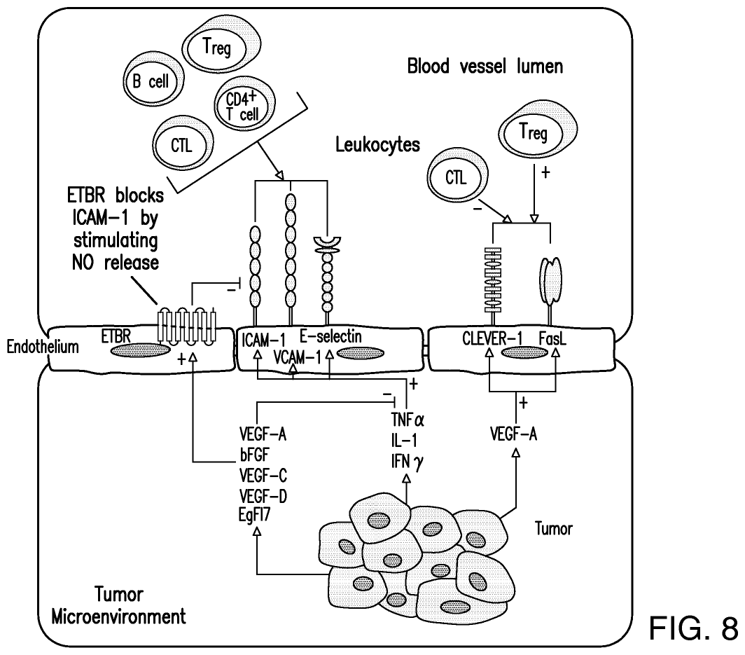

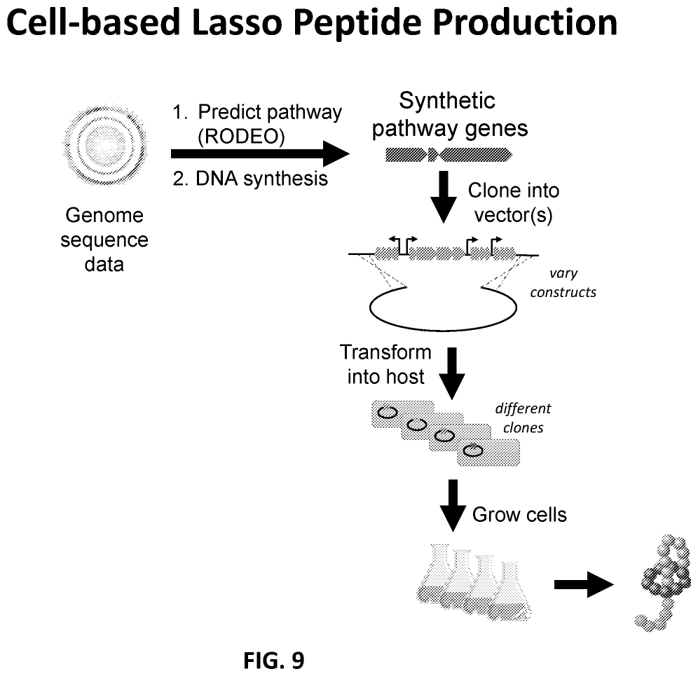

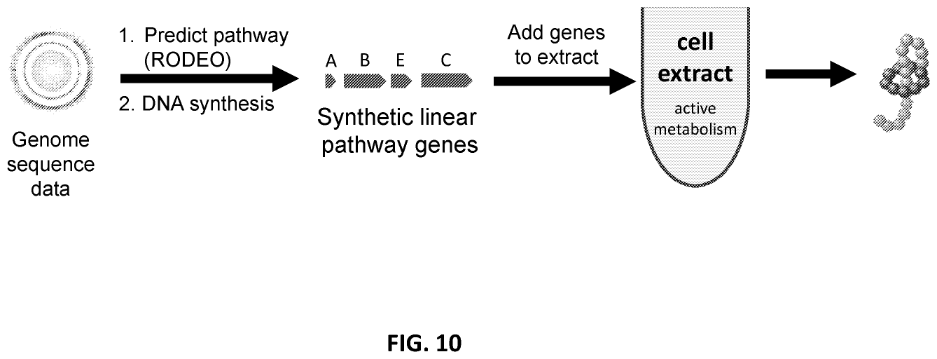

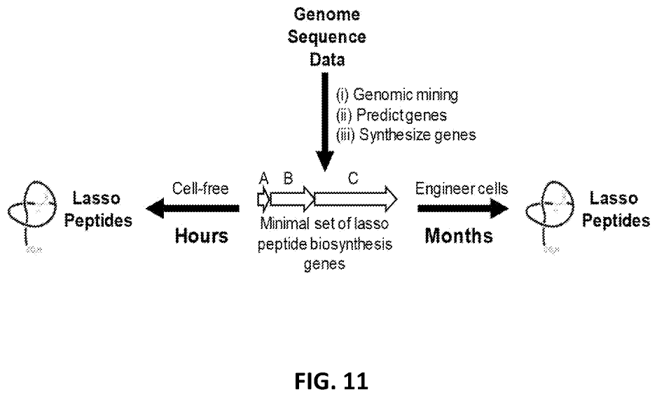

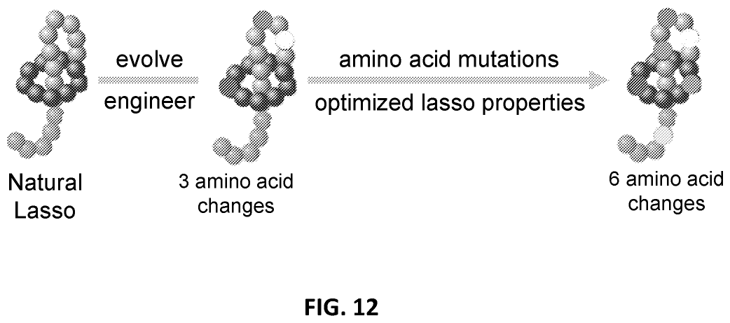

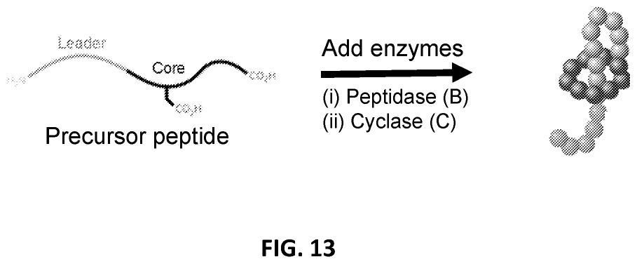

ET-1 Receptor Binding Assay. The reaction mixture (1 ml) containing 0.74 kBq/ml [¹²⁵I]ET-1, 50 mM Tris-HCl buffer (pH 7.6), 1 mM EDTA, 0.2% bovine serum albumin (BSA), 0.02% bacitracin, 14 mg of CHO cell membranes expressing ETAR or ETBR, and various concentration of lasso peptides (having amino acid sequences of SEQ ID NOS:1-4) is incubated at room temperature for 2 hours, then filtered through GF/B glass filters. The glass filters are washed three times with cold 50 mM Tris-HCl buffer containing 1 mM EDTA. The radioactivity on the washed filter is measured by using a Packard γ counter. Non-specific binding is measured in the presence of 0.1 mM unlabeled ET-1. Binding affinity is calculated using non-linear regression analysis of the amount of radiolabeled drug bound to the membrane, based upon radioactivity measurements determined upon treatment with different concentrations of unlabeled lasso peptide (having amino acid sequences of SEQ ID NOS:1-4). Inhibition data for Lasso 1 vs ETAR and ETBR is illustrated in the graphs in FIG. 4 and IC₅₀ data for lasso peptides having primary sequences of SEQ ID NOS: 1-4, 42-54, 18 and 20 vs ETBR is shown in following Table 4.

TABLE 4

| SEQ | | ETBR |
|---|---|---|
| ID NO: | A.A. Sequence | IC₅₀ (nM) |
| 1 | GNWHGTAPDWFFNYYW | 10 |
| 2 | GNWHGTAPDWFFNYYW-7-OH | 22 |
| 3 | GNWHGTSPDWFFNYYW | 5 |
| 4 | GNWHGTSPDWFFNYYW-7-OH | 8 |
| 42 | GNWHGTAPDWFFNYY | 54 |
| 43 | GNWHGTAPDWFFNYYWW | 36 |
| 44 | GNWHGTAPDWFFNYYA) | 470 |
| 45 | GNWHGTAPDWFFNYYF | 12 |
| 46 | GNWHGTAPDWFFNYYY | 45 |
| 47 | GNWHGTAPDWFFNYYNYYW | 810 |
| 48 | GNWHGTAPDWFFNYYNIIW | >70 |
| 49 | GNWHGTAPDWFFNYYAHLDIIW | >45 |
| 50 | GNWHGTAPDWFFNYYTrn | 50 |
| 51 | GNWHGTAPDWFFNYYW-OMe | 15 |
| 52 | GNWHGTAPDWFFNYYW-OBn | 140 |
| 53 | GNWHGTAPDWFFNYYW-NH2 | 49 |
| 54 | GNWHGTAPDWFFNYYNal | 6.3 |
| 18 | MTEQSEQTPAEYIPPMLVEVGE | >5000 |
| | FTEDTLGNWHGTAPDWFFNYYW | |
| 20 | MTEQSEQTPAEYIPPMLVEVGE | >5000 |
| | FTEDTLGNWHGTSPDWFFNYYW | |

### Example 6. Cancer Cell Line Growth Inhibition by Lasso 1 and Lasso 3

ETBR antagonistic lasso peptides Lasso 1 having the primary amino acid sequence of SEQ ID NO: 1 and Lasso 3 having the primary amino acid sequence of SEQ ID NO: 3 are tested for their effect in inhibition of cancer cell growth. Cancer cell lines used for growth and proliferation inhibition assays are obtained from commercial sources (Life Technologies, Carlsbad, Calif. or ATCC, Monasses, Va.). All cell lines are stored and maintained in recommended media containing 10% fetal bovine serum (Thermo Fisher, Waltham, Mass.) according to provider's instructions. Cancer cell lines for use to screen lasso peptides of Lasso 1 and Lasso 3 for growth inhibition activity include:

### Pancreatic: MIA PaCa-2, PANC-1, Capan-1, PSN1, and JOPACA-1

### Colorectal: Caco-2, COLO 320, DLD-1, HCT-15, HCT-116, HT-29, and SW48

### Gastric: AGS, SNU-1, SNU-5, SNU-16, Hs 746T, NCI-N87, KATO III, HGC-27, MNK28, MNK45

### Hepatic: HepG2, C3A, HuH7, Hep3B, HLE, HepaRG, HLF, SK-Hepl, PLC/PRF/5

### Prostate: DU-145, PC-3 and LNCaP, LAPC-4, LAPC-9, and VCaP

### Melanoma: RPMI-7951, SK MEL 28, SK MEL 31, SK MEL 24, A375

### Glioblastoma: U-87 MG, U-118 MG, U-138 MG, LN-18, LN-229

Ovarian Cancer: PA-1, Caov-3, SW 626, SK-OV-3, UWB1.289, ID8 (mouse)

Cancer Cell Growth Inhibition Data:

Lasso peptides of this disclosure of Lasso 1 and Lasso 3 are tested for growth inhibition against cancer cell lines, including the melanoma cell lines RPMI-7951, SK MEL 28, SK MEL 31, SK MEL 24, A375, and live cells are quantified using either the MTS assay [3-(4,5-dimethylthiazol-2-yl)-5(3-carboxymethoxyphenyl)-2-(4-sulfophenyl)-2H-tetrazolium, inner salt] by measuring OD at 492 nm according to manufacturer's instructions or the Cell Titer-Glo® 2.0 Luminescent Cell Viability Assay (Promega Corporation, Madison, Wis., USA). The Cell Titer-Glo® 2.0 Luminescent Cell Viability Assay is a sensitive homogeneous method to determine the number of viable cells in culture. Detection is based on using the luciferase reaction to measure the amount of ATP from viable cells. The amount of ATP in cells correlates with cell viability. Within minutes after a loss of membrane integrity, cells lose the ability to synthesize ATP, and endogenous ATPases destroy any remaining ATP; thus the levels of ATP fall precipitously. The Cell Titer-Glo® 2.0 Reagent does three things upon addition to cells. It lyses cell membranes to release ATP; it inhibits endogenous ATPases, and it provides luciferin, luciferase and other reagents necessary to measure ATP using a bioluminescent reaction. The unique properties of a proprietary stable luciferase. There is a linear relationship (r2=0.99) between the luminescent signal and the number of cells from 0 to 50.000 cells per well.

Reagents Used

### DMEM (Paneco, cat # C420)

### Williams Medium (Gibco, cat #12551032)

Fetal bovine serum, FBS (HyClone, cat # SH 30084.03)

### Pen-Strep (Paneco, cat # A065)

MEM non-essential amino acids (Paneco, cat # F115)

### L-glutamine (Paneco, cat # F032)

Sodium pyruvate (Paneco, cat # F023)

### Versen (Paneco, cat # P080)

### Accutase (Innovative Cell Technologies, Inc., cat. # AT104)

### DMSO (Panreac, cat. #141954.1611)

### The Cell Titer-Glo® 2.0 Luminescent Cell Viability Assay (Promega, cat. # G9241)

Equipment and Materials Used

### Biomek 384 FX Laboratory Automated Workstation (Beckman Coulter Inc., Fullerton, Calif.)

### Biomek 2000 Laboratory Automated Workstation (Beckman Coulter Inc., Fullerton, Calif.)

### Microscope Axiovert 40

Microbiological safety cabinet, classII (NuAire, USA)
CO₂ incubator (VWR Science, USA)
Bright line Hemacytometer (Z359629, Sigma, IL, USA)
Tecan Infinite M1000 microplate reader (Thermo Fisher, Waltham, Mass.)
Plates: 384-well black/clear, tissue culture treated, flat bottom (Falcon, #353962)

Growth Inhibition Studies Using Melanoma Cell Lines

### RPMI-7951

### SK MEL 28,

### SK MEL 31,

### SK MEL 24,

### A375

Human melanoma cell lines RPMI-7951, SK MEL 28, SK MEL 31, SK MEL 24, and A375, and the human kidney cell line HEK-293, are obtained from the American Tissue Culture Collection (ATCC, Manassas, Va., USA) and are cultured with DMEM (ATCC) containing 10% FCS (Gemini Biological Products, Calabasas, Calif.), 2 mM glutamine (GIBCO-BRL), and 100 mg/ml antibiotics (Pen-Strep mix (GIBCO-BRL) in a humidified incubator with 5% CO2 at 37° C.

Cell Propagation

Conditions: 37° C., air 95%; HEPA-filtered carbon dioxide (CO2) 5%, humidified atmosphere.
Cells are grown in 175 cm² flasks to 80-90% of confluency.
Culture media are aspirated and cell layer is briefly rinsed with Versen solution to remove all traces of serum.
2 mL of Accutase is added to cells.
Flasks are returned to incubator for 5 min to allow cell detachment.
Add 6.0 mL of complete growth medium.
Single cell suspension is created by gently pipetting.
Cells are counted using a Hematocytometer and a suspension with the desired cell concentration is prepared.

Cell Plating
Single cell suspensions are prepared as described above, cells are recounted and resuspended to final density.
Cells are plated in a 384-well optical bottom plates by Biomek 384 FX, with 40 μL of cell suspension in each well.
Assay plates are centrifuged at 100 rpm for 1 minute and then kept at 37° C., 5% CO2, humidified atmosphere for 24 hours prior to treatment.

To initiate the melanoma cell growth inhibition study, lasso peptides of this disclosure of Lasso 1 and Lasso 3, and reference compounds BQ-788 and BQ-123 (Sigma-Aldrich, St Louis, Mo.) are dissolved in DMSO solution to create 10 mM stock solutions. Stock solutions (70 μL) are diluted with cell culture media to produce 500× serial dilutions and 10 of final compound dilutions are added to cells in assay plates to screen a series of 10 doses starting at 100 mM. Final DMSO concentration is 0.2%. Assay plates are centrifuged at 100 rpm, 1 minute and kept at 37° C., 5% CO2, humidified atmosphere. After 72 hours of incubation with compounds, 10 μL CellTiter-Glo Reagent is added to assay plates using the Biomek 384 FX. Luminescence intensity is measured for each well using the Tecan M 1000 microplate reader after 5 min of incubation with the Cell Titer-Glo® 2.0. The number of viable cells in culture is determined based on quantitation of the ATP present in each culture well. Experimental data is calculated as percent growth inhibition by dividing luminescence values from treated wells by the average luminescence values from untreated control wells and subtracted from 100. The EC₅₀ value is defined as the drug concentration needed to inhibit 50% of the cell growth compared to growth of the untreated control cells. The EC₅₀ curves are plotted and EC₅₀ values are calculated using the GraphPad Prism 4 program based on a sigmoidal dose-response equation. P values are calculated by using the Student's t test for absolute values. Growth of all tested human melanoma cell lines is found to be inhibited by Lasso 1, Lasso 3, and BQ-788, but not by BQ123. Growth of the HEK-293 cell line is not inhibited by any of the compounds tested.

### Example 7. Treatment of Melanoma by Administration of Lasso 3 in a Melanoma Xenograft Animal Model

Animal models are used to examine the ability of a compound of this disclosure to ameliorate the growth of melanoma. Inhibitors of this disclosure are applied in mice where melanoma tumors are induced through xenografts created by the transfer of melanoma cancer cells, such as RPMI-7951 or A375, into the subcutaneous or peritoneal space of a mouse, or through production of a human-mouse melanoma tumor xenograft.

Mice. Thirty female BALB/c nude mice are purchased from The Jackson Laboratory (Bar Harbor, Me., USA). Animals are maintained in accordance with the Guide for the Care and Use of Laboratory Animals,

Compounds: Lasso 3 is prepared for in vivo intraperitoneal administration in PBS vehicle (phosphate-buffered saline: Thermo Fisher Scientific Inc., Waltham, Mass.) containing 0.5% methylcellulose/0.025% Tween 20 (Sigma-Aldrich, St. Louis, Mo.).

Melanoma Xenograft Mice

Dissociated A375 melanoma cells are suspended in Hank's balanced salt solution (HBSS) (2×10⁶ cells/mL), and the suspension (100 μL) is subcutaneously injected into the back of six female BALB/c nude mice. Mice are maintained for 15 days following injection of melanoma cells, until the tumors develop and reach at least 20-25 mm in diameter (measured by caliper), after which the mice are euthanized, the tumors are excised, cut into pieces of approximately 4-6 mm in diameter, and implanted under the flank skin of a new series of twenty-four female BALB/c nude mice. When a tumor reaches a volume of 100 mm³ (12-15 days), the mice are randomly split into three groups of six treatment mice and one vehicle control group. After establishment of the nude mouse xenograft model, tumor dimensions are measured every 2 days using micrometer calipers. Lasso 3 is administered to three groups intraperitoneally, at doses of 25 mg/kg, 50 mg/kg and 100 mg/kg daily for 5 consecutive days, followed by 2 days with no injections, and the cycle is then repeated three times. The control group of six mice receives vehicle only, similarly once per day (q. d.) for 5 consecutive days, followed by 2 days with no injections, and the cycle is then repeated three times. Mice are weighed daily, starting from the date of A375 cell injection (day 0). If a tumor reaches >2000 mm³ or shows significant loss of body weight, the animal is euthanized according to ethical guidelines. Three days after the end of treatment, the mice are euthanized via carbon dioxide asphyxiation and all tumors are excised immediately following death, weighed and measured, and then snap-frozen in liquid nitrogen. Tumor volume (TV) is calculated by the following formula: TV=0.5ab², where a is tumor length in mm, and b is tumor width in mm. Lasso 3 is shown to reduce tumor size in a dose-dependent manner from 20-88% relative to control tumors (vehicle) and mouse body weight is maintained within 10% of day 0 baseline.

### Example 8. Treatment of Ovarian Cancer by Administration of Lasso 3 in Combination with a Cell-Based Tumor Vaccine in an ID8 Xenograft Animal Model of Ovarian Cancer

Thirty 6-8-week-old female C57BL/6 syngeneic mice (Charles River Labs, Wilmington, Mass., USA) are vaccinated by two subcutaneous injections of 5×10⁶ UV-irradiated ID8 mouse ovarian cancer cells 1 week apart. One week after the second vaccination, the mice are inoculated with 5×10⁶ ID8 murine ovarian cancer cells subcutaneously in the flank with 300 μl Matrigel or intraperitoneally (i.p.) with 300 μl PBS in the C57BL/6 syngeneic mice (see: Roby, K. F., et al., Carcinogenesis, 2000, 21(4), 585-591). Tumors are established with a volume of 100 mm³ after 14 days following inoculation with ID8 cell and the mice are randomly assigned to six groups of five mice. On day 15, mice with sc and ip tumors are treated separately with Lasso 3 (100 mg/kg) in PBS containing 0.5% methylcellulose/0.025% Tween 20 by ip injection, a control 22-amino acid scrambled peptide (SEKDREGVRIRERGAVRKSLPA (SEQ ID NO:72); ProSci), and a vehicle control (PBS containing 0.5% methylcellulose/0.025% Tween 20), administered daily for 15 d, after which tumors are harvested for molecular, cellular, histopathology analysis. Vaccine combined with Lasso 3 leads to a 70-90% decrease in tumor growth and 30-50% longer tumor-free survival, relative to vaccine plus control peptide or vehicle control alone.

### Example 9. Treatment of Ovarian Cancer by Administration of Lasso 3 in Combination with a DNA-Based Tumor Vaccine in a TC-1 Xenograft Animal Model of Ovarian Cancer

Twenty 6-8-week-old female C57BL/6 syngeneic mice (Charles River Labs, Wilmington, Mass., USA) are inoculated in the flank with 3×10⁴ cells suspended in 100 μL PBS. Four days after tumor inoculation, vaccination is performed by two intramuscular injections of 50 μg pcDNA3.1-LLO-E7 vector (Peng, X., et al., Cancer Immunol. Immunother. 2007, 56(6), 797-806) expressing human papilloma virus-16 E7 gene (in 50 μl PBS). Seven days after the second vaccination, tumors are treated separately with Lasso 3 (100 mg/kg) in PBS containing 0.5% methylcellulose/0.025% Tween 20 by ip injection, a control 22-amino acid scrambled peptide (SEKDREGVRIRERGAVRKSLPA (SEQ ID NO:72); ProSci), and a saline control, each administered daily for 15 d, after which tumors are harvested for molecular, cellular, histopathology analysis. Vaccine combined with Lasso 3 leads to a 75-90% decrease in tumor growth and 40-60% longer tumor-free survival, relative to vaccine plus control peptide or vehicle control alone.

### 7. SEQUENCE LISTING

The present specification is being filed with a computer readable form (CRF) copy of the Sequence Listing. The CRF entitled 14619-007-228_SEQLIST.txt, which was created on Jan. 3, 2021 and is 40,281 bytes in size, is identical to the paper copy of the Sequence Listing and is incorporated herein by reference in its entirety.

### REFERENCES

American Cancer Society. Cancer Facts & Figures 2019. Atlanta: American Cancer Society; 2019.
Vignon-Zellweger, N., et al., Endothelin and endothelin receptors in the renal and cardiovascular systems, Life Sciences, 2012, 91, 490-500.
Mazzuca, M. Q., Khalil, R. A. Vascular endothelin receptor type B: structure, function and dysregulation in vascular disease, Biochem Pharmacol. 2012; 84(2): 147-162.
Rosano, L., Bagnato, A., Endothelin therapeutics in cancer: Where are we? Am J Physiol Regul Integr Comp Physiol, 2016, 310: R469-R475.
Bagnato, A., et al, Role of the endothelin axis and its antagonists in the treatment of cancer, Brit. J. Pharmacol., 2011, 163, 220-233.
Lahav, R., et al., An endothelin receptor B antagonist inhibits growth and induces cell death in human melanoma cells in vitro and in vivo, Proc. Natl. Acad. Sci., 1999; 96: 11496-11500.
Ishimoto, S., et al., Role of endothelin receptor signalling in squamous cell carcinoma, Int. J. Oncology, 2012, 40, 1011-1019.
Tanaka, T,, et at, Endothelin B receptor expression correlates with tumour angiogenesis and prognosis in oesophageal squamous cell carcinoma, Brit. J. Cancer, 2014, 110, 1027-1033.
Grimshaw, M. J., et al., A Role for Endothelin-2 and Its Receptors in Breast Tumor Cell Invasion, Cancer Res., 2004, 64, 2461-2468.
Wulfing, P., et al, Expression of endothelin-1, endothelin-A, and endothelin-B receptor in human breast cancer and correlation with long-term follow-up, Clin. Cancer Res., 2003, 9, 4125-4131,
Vasaiker, S., et al., Overexpression of endothelin B receptor in glioblastoma: a prognostic marker and therapeutic target? BMC Cancer, 2018, 18: 154.
Wan, X., et al., Role of endothelin B receptor in oligodendroglioma proliferation and survival, in vitro and in vivo evidence, Mol. Med. Rep., 2014, 9: 229-234.
Wulfing, C., et al., Expression of the endothelin axis in bladder cancer: relationship to clinicopathologic parameters and long-term survival, Eur. Urol., 2005, 47(5), 593-600
Awano, S., et al., Endothelin system in oral squamous carcinoma cells: Specific siRNA targeting of ECE-1 blocks cell proliferation, Int. J. Cancer, 2006, 118, 1645-1652.
Eltze, E., et al., Expression and prognostic relevance of endothelin-B receptor in vulvar cancer, Oncology Rep., 2007, 18, 305-311.
Wuttig, D., et al., CD31, EDNRB and TSPAN7 are promising prognostic markers in clear-cell renal cell carcinoma revealed by genome-wide expression analyses of primary tumors and metastases, Int. J. Cancer, 2012, 131, E693-E704.
Russignan, A., et al., Endothelin-1 receptor blockade as a new possible therapeutic approach in multiple myeloma, British Journal of Haematology, 2017, 178, 781-793.
Cook, N., et al., Endothelin-1 and endothelin B receptor expression in pancreatic adenocarcinoma. J. Clin. Pathol., 2015, 68(4), 309-313.
Rosano, R., et al., Endothelin receptor blockade inhibits molecular effectors of Kaposi's sarcoma cell invasion and tumor growth in vivo, Am. J. Pathology, 2003, 163(2), 753-762.
Buckanovich, R. J., et al., Endothelin B receptor mediates the endothelial barrier to T cell homing to tumors and disables immune therapy, Nature Med., 2008; 14(1): 28-36.
Coffman, L., et al., Endothelin receptor A is required for the recruitment of antitumor T cells and modulates chemotherapy induction of cancer stem cells. Cancer Biol. Ther., 2013, 14(2), 184-192.
Shaul, M. E., Fridlender, C. G., Tumour-associated neutrophils in patients with cancer, Nature Rev. Clin. Oncol., 2019, 16, 601-620.
Zarpelon, A. C., et al., Endothelin-1 induces neutrophil recruitment in adaptive inflammation via TNFα and CXCL1/CXCR2 in mice. Canadian J. Physiol. and Pharmacol., 2012, 90(2), 187-199.
Facciabene, A., et al., Local endothelial complement activation reverses endothelial quiescence, enabling T-cell homing, and tumor control during T cell immunotherapy, Oncoimmunology, 2017, 6(9), e1326442.
Georganski, M., et al., Vascular targeting to increase the efficiency of immune checkpoint blockade in cancer, Front. Immunol, 2018, 9:3081.
Aubert, J., et al., Endothelin receptor antagonists beyond pulmonary arterial hypertension, cancer and fibrosis, J. Med. Chem. 2016, 59, 8168-8188.
Davenport, A. P., et al., New drugs and emerging targets in endothelin signaling pathway and prospects for precision medicine, Physiol. Res., 2018, 67 (Suppl. 1), S37-S54. Ishikawa, K. et al., Biochemical and pharmacological profile of a potent and selective endothelin B-receptor antagonist, BQ-788, Proc. Nat. Acad. Sci. USA, 1994, 91, 4892-4896.
He, J. X., et al., An efficient preparation of the pseudopeptide endothelin-B receptor selective antagonist BQ-788. J. Org. Chem., 1995, 60, 8262-8266.
Morishita, Y., et al., RES-701-1, a novel and selective endothelin type B receptor antagonist produced by Streptomyces spp. RE-701. J. Antibiotics, 1994, 47(3), 269-275.
Ogawa, T., et al., RES-701-2, -3, -4, novel and selective endothelin type B receptor antagonists produced by Streptomyces sp. J. Antibiotics, 1995, 48(11), 1213-1220.
Karaki, H., Matsuda, M. RES-701-1, a novel endothelin ETB receptor antagonist, Cardiovascular Drug Rev., 1996, 14, 17-35.
Zhang, Y., et al, Heterologous production of microbial ribosomally synthesized and post-translationally modified peptides, Front. Microbiol., 2018, doi: 10.3389/fmicb.2018.01801.
Maguire, J. J., Davenport, A. P., Endothelium receptors and their antagonists, Sem. Nephrology, 2015, 35(2), 125-136.
Cabrera-Vera, T. M., et al., Insights into G Protein Structure, Function, and Regulation. Endocr. Rev. 2003, 24, 765-781.
Breu, V., et al., In vitro characterization of Ro 46-8443, the first non-peptide antagonist selective for the endothelin ET-B receptor. FEBS Lett., 1996, 383, 37-41.

### Above

Balwierczak, J. L., et al., Characterization of a Potent and Selective Endothelin-B Receptor Antagonist, IRL 2500. J. Cardiovasc. Pharmacol., 1995, 26 (Suppl.3), S393-S396.
von Geldern, T. W., et al., Pyrrolidine-3-carboxylic Acids as Endothelin Antagonists. 4. Side Chain Conformational Restriction Leads to ETB Selectivity, J. Med. Chem. 1999, 42, 3668-3678.
Liu, G., et al, Design, Synthesis, and Activity of a Series of Pyrrolidine-3-carboxylic Acid-Based, Highly Specific, Orally Active ETB Antagonists Containing a Diphenylmethylamine Acetamide Side Chain. J. Med. Chem. 1999, 42, 3679-368.
Cowburn, P. J., et al., Comparison of selective ETA and ETB receptor antagonists in patients with chronic heart failure, Eur. J. Heart Failure, 2005, 7, 37— 42.
Strachan, F. E., et al., Systemic Blockade of the Endothelin-B Receptor Increases Peripheral Vascular Resistance in Healthy Men, Hypertension, 1999, 33, 581-585.
Verhaar, M. C., et al., Endothelin-A Receptor Antagonist-Mediated Vasodilatation Is Attenuated by Inhibition of Nitric Oxide Synthesis and by Endothelin-B Receptor Blockade, Circulation, 1998, 97, 752-756.
Zuccarello, M., et al., Endothelin B Receptor Antagonists Attenuate Subarachnoid Hemorrhage-Induced Cerebral Vasospasm, Stroke, 1998, 29, 1924-1929.
Tietz, J. I., et al., A new genome-mining tool redefines the lasso peptide biosynthetic landscape, Nature Chem Bio, 2017, 13, 470-478.
Hegemann, J. D., et al., Lasso Peptides from Proteobacteria: Genome Mining Employing Heterologous Expression and Mass Spectrometry, Biopolymers, 2013, 100, 527-542.
Hegemann, J. D., et al., Lasso Peptides: An Intriguing Class of Bacterial Natural Products, Acc. Chem. Res., 2015, 48, 1909-1919.
Maksimov, M. O., et al., Precursor-centric genome-mining approach for lasso peptide discovery, Proc. Nat. Acad. Sci., 2012, 109, 15223-15228.
Maksimov, M. O., et al., Lasso peptides: structure, function, biosynthesis, and engineering, Nat. Prod. Rep., 2012, 29, 996-1006.
Zhao, N., et al., Lasso peptide, a highly stable structure and designable multifunctional backbone, Amino Acids, 2016, 48, 1347-1356.
Zong, C., et al., Construction of Lasso Peptide Fusion Proteins, ACS Chem. Biol., 2016, 11, 61-68.
Burkhart, B. J., et al., A prevalent peptide-binding domain guides ribosomal natural product biosynthesis, Nat. Chem. Biol., 2015, 11, 564-570.
Li, Y.; Rebuffat, S.; Zirah, S., Lasso Peptides, Springer Press: New York, 2015.
DiCaprio, A. J., et al., Enzymatic Reconstitution and Biosynthetic Investigation of the Lasso Peptide Fusilassin, J. Am. Chem. Soc., 2019, 141, 290-297.
Pavlova, O., et al., Systematic Structure-Activity Analysis of Microcin J25, J. Biol. Chem., 2008, 283, 25589-25595
Knappe, T. A., et al., Insights into the Biosynthesis and Stability of the Lasso Peptide Capistruin, Chem. Biol., 2009, 16, 1290-1298
Fage, C. D., et al., Structure and Mechanism of the Sphingopyxin I Lasso Peptide Isopeptidase, Angew. Chem. Int. Ed., 2016, 55, 12717-12721.
Al Toma, R. S., et al., Site-Directed and Global Incorporation of Orthogonal and Isostructural Noncanonical Amino Acids into the Ribosomal Lasso Peptide Capistruin, ChemBioChem, 2015, 16, 503-509.
Blin, K., et al., antiSMASH 4.0—improvements in chemistry prediction and gene cluster boundary identification, Nucleic Acids Research, Volume 45, Issue W1, 3 Jul. 2017, Pages W36-W41
Ibrahim, A., et al., Dereplicating nonribosomal peptides using an informatics search algorithm for natural products (iSNAP) discovery, Proc. Nat. Acad. Sci., USA., 2012, 109, 19196-19201.
Starcevic, A., et al., ClustScan: an integrated program package for the semi-automatic annotation of modular biosynthetic gene clusters and in silico prediction of novel chemical structures, Nucleic Acids Res., 2008, 36, 6882-6892.
Tomobe Y, Miyauchi T, Saito A, Yanagisawa M, Kimura S, Goto K, Masaki T., Effects of endothelin on the renal artery from spontaneously hypertensive and Wistar Kyoto rats, Eur. J. Pharmacol., 1988, 152(3): 373-374.
Lehrke I, Waldherr R, Ritz E, Wagner J., Renal endothelin-1 and endothelin receptor type B expression in glomerular diseases with proteinuria, J Am Soc Nephrol., 2001, 12(11): 2321-2.329.
Feldstein C. Romero C Role of endothelins in hypertension. Am J Ther., 2007, 14(2): 147-153.
Iglarz M, Clozel M., Mechanisms of ET-1-induced endothelial dysfunction. J Cardiovasc Pharmacol., 2007, 50(6): 621--628.
Spinella F., et al., Endothelin-1 stimulates lymphatic endothelial cells and lymphatic vessels to grow and invade. Cancer Res., 2009, 69(6): 2669-2676.
Nelson J, Bagnato A, Battistini B, Nisen P The endothelin axis: emerging role in cancer. Nat Rev Cancer, 2003, 3(2): 110-116.
Herrmann E. et al., The endothelin axis in urologic tumors: mechanisms of tumor biology and therapeutic implications. Expert Rev Anticancer Ther, 2006, 6(1): 73-81.
Wulfing P, et al., Endothelin-1 endothelin-A-, and endothelin-B-receptor expression is correlated with vascular endothelial growth factor expression and angiogenesis in breast cancer. Clin Cancer Res., 2004, 10(7): 2393-2400.
Bagnato A., et al., The endothelin axis in cancer: the promise and the challenges of molecularly targeted therapy. Can J Physiol Pharmacol., 2008, 86(8):173-484.
Goering, A. W., et al., In Vitro Reconstruction of Nonribosomal Peptide Biosynthesis Directly from DNA Using Cell-Free Protein Synthesis, ACS Synth Biol., 2017, 6(1), 39-44.
Kay, J., et al., Lysate of engineered Escherichia coli supports high-level conversion of glucose to 2,3-butanediol, Metabolic Engineering, 2015, 32, 133-142
Zimmerman, E. S., et al., Production of Site-Specific Antibody-Drug Conjugates Using Optimized Non-Natural Amino Acids in a Cell-Free Expression System, Bioconjugate Chem., 2014, 25, 351-361
Hodgman, C. E., Jewett, M. C., Cell-Free Synthetic Biology: Thinking Outside the Cell, Metab. Eng., 2012, 14(3), 261-269.
Carlson, E. D., et al., Cell-free protein synthesis: Applications come of age, Biotechnol. Adv., 2012, 30(5), 1185-1194.
Sun, Z. Z., Hayes, C. A., Shin, J., Caschera, F., Murray, R. M., Noireaux, V. Protocols for Implementing an Escherichia coli Based TX-TL Cell-Free Expression System for Synthetic Biology, J. Vis. Exp. 2013, 79, e50762, doi:10.3791/50762
Gagoski, D., et al., Performance Benchmarking of Four Cell-Free Protein Expression Systems, Biotechnol. Bioeng. 2016; 113: 292-300.
Krinsky, N., et al., A Simple and Rapid Method for Preparing a Cell-Free Bacterial Lysate for Protein Synthesis, PLoS ONE, 2016, 11(10): e0165137.
Wang, Y., et al., Cell-free protein synthesis energized by slowly-metabolized maltodextrin, BMC Biotechnology, 2009, 9:58 doi:10.1186/1472-6750-9-58.
Hoffmann, M., et al., Rapid translation system: A novel cell-free way from gene to protein, Biotech. Ann. Rev., 2004, 10, 1-29.
Gagoski, D., et al., Performance Benchmarking of Four Cell-Free Protein Expression Systems, Biotechnol. Bioeng., 2016; 113: 292-300.
Shimizu, Y., et al PURE Technology, Y. Endo et al. (eds.), Cell-Free Protein Production: Methods and Protocols, in Methods in Molecular Biology, vol. 607, Chapter 2, pp 11-21, Springer: New York, 2010.
Takai, K, et al., Practical cell-free protein synthesis system using purified wheat embryos, Nature Protocols, 2010, 5, 227-238.
Li, J., et al., Improved Cell-Free RNA and Protein Synthesis System, PLoS ONE, 2014, 9, e106232. doi:10.1371/journal.pone.0106232
Kigawa, T., et al., Preparation of Escherichia coli cell extract for highly productive cell-free protein expression, J. Struct. Functional Genomics, 2004, 5, 63-68.
Josephson, K., et al., mRNA display: from basic principles to macrocycle drug discovery, Drug Discov. Today, 2014, 19, 388-399
Doi, N., et al., DNA Display Selection of Peptide Ligands for a Full Length Human G Protein-Coupled Receptor on CHO-K1 Cells, PLoS ONE, 2012, 7, e30084, pp 1-8.
Gamkrelidze, M., Dabrowska, K., T4 bacteriophage as a phage display platform, Arch Microbiol, 2014, 196, 473-479.
Josephson, K., et al., Ribosomal Synthesis of Unnatural Peptides, J. Am. Chem. Soc., 2005, 127, 11727-11735
Cherf, G. M., Cochran, J. R., Applications of yeast surface display for protein engineering, Methods Mol Biol., 2015, 1319, 155-175
Franzini, R. M., et al., DNA-Encoded Chemical Libraries: Advancing beyond Conventional Small-Molecule Libraries, Acc. Chem. Res., 2014, 47, 1247-1255
Kretz, K. A., et al., Gene site saturation mutagenesis: a comprehensive mutagenesis approach, Methods Enzymol., 2004, 388, 3-11
Nannemann, D. P, et al., Assessing directed evolution methods for the generation of biosynthetic enzymes with potential in drug biosynthesis, Future Med Chem., 2011, 3, 809-819
Fox, R. J., et al., Enzyme optimization: moving from blind evolution to statistical exploration of sequence-function space, Trends Biotechnol., 2008, 26, 132-138.
Fox, R. J., et al., Improving catalytic function by ProSAR-driven enzyme evolution, Nature Biotechnol., 2007, 25, 338-344
Odegrip, R., et al., CIS display: In vitro selection of peptides from libraries of protein-DNA complexes, Proc. Nat. Acad. Sci. U.S.A., 2004, 101, 2806-2810.
Ullman, C. G., et al., In vitro methods for peptide display and their applications, Briefings Functional Genomics, 2011, 10, 125-134.
Tan, G.-Y., et al., Rational synthetic pathway refactoring of natural products biosynthesis in actinobacteria, Metabolic Engineering, 2017, 39, 228-236.
Shin, D.-S., et al., Combinatorial Solid Phase Peptide Synthesis and Bioassays, J. Biochem. Mol. Bio., 2005, 38, 517-525.
Presolski, S. I., et al., Copper-Catalyzed Azide-Alkyne Click Chemistry for Bioconjugation, Curr Protoc Chem Biol., 2011, 3, 153-162
Cromm, P. M., et al., Orthogonal ring-closing alkyne and olefin metathesis for the synthesis of small GTPase-targeting bicyclic peptides, Nat. Comm., 2016, 7, 11300.
Gleeson, E. C., et al., Ring-closing metathesis in peptides, Tetrahedron Lett., 2016, 57, 4325-4333.
Karlin, S. and Altschul, S. F., Methods for assessing the statistical significance of molecular sequence features by using general scoring schemes, Proc. Natl. Acad Sci. USA, 1990, 87:2264-2268.
Karlin, S. and Altschul S. F., Applications and statistics for multiple high-scoring segments in molecular sequences, Proc. Natl. Acad Sci. USA, 1993, 90:5873-5877
Altschul, S. F., et al., Basic local alignment search tool, J. Mol. Biol., 1990, 215, 403-410
Altschul, S. F., et al., Gapped BLAST and PSI-BLAST: a new generation of protein database search programs, Nucleic Acids Res., 1997, 25, 3389-3402.
Henikoff, S., et al., Amino acid substitution matrices from protein blocks, Proc. Natl. Acad Sci. USA, 1992, 89, 10915-1091.
Ohtsuka, E. et al., An alternative approach to deoxyoligonucleotides as hybridization probes by insertion of deoxyinosine at ambiguous codon positions, J. Biol. Chem., 1985, 260, 2605-2608
Rossolini, G. M., et al., Use of deoxyinosine-containing primers vs degenerate primers for polymerase chain reaction based on ambiguous sequence information, Mol. Cell. Probes, 1994, 8, 91-98
Holliger, P. et al., Engineered antibody fragments and the rise of single domains, Nature Biotech. 2005, 23 (9), 1126-1129
Rostovtsev, V. V, et al., A stepwise Huisgen cycloaddition process: copper(I)-catalyzed regioselective “ligation” of azides and terminal alkynes, Angew. Chem. Int. Ed. Engl., 2002, 41, 2596-2599
Sun, X.-L., et al., Carbohydrate and Protein Immobilization onto Solid Surfaces by Sequential Diels—Alder and Azide—Alkyne Cycloadditions, Bioconjugate Chem., 2006, 17, 52-57.
Roby, K. F., et al., Development of a syngeneic mouse model for events related to ovarian cancer, Carcinogenesis, 2000, 21(4), 585-591.
Peng, X., et al., Adjuvant properties of listeriolysin O protein in a DNA vaccination strategy, Cancer Immunol. Immunother. 2007, 56(6), 797-806.

1. A method of managing, preventing, or treating an endothelin B receptor (ETBR)-mediated proliferative disease producing neoplastic cells in a subject, comprising administering to the subject a therapeutic effective amount of a lasso peptide, wherein the lasso peptide comprises an amino acid sequence selected from SEQ ID NOS:1-17 and 42-56.

2. The method of claim 1, wherein the lasso peptide is G1-D9 cyclized.

3. The method of claim 1 or 2, wherein the lasso peptide competes with endothelin for the binding with ETBR.

4. The method of claim 3, wherein the endothelin is endothelin 1, endothelin 2 and/or endothelin 3.

5. The method of any one of claims 1 to 3, wherein upon administration, the lasso peptide preferentially binds to ETBR over endothelin A receptor (ETAR).

6. The method of claim 5, wherein upon administration, the lasso peptide specifically inhibits ETBR.

7. The method of any one of claims 1 to 6, wherein upon administration, the lasso peptide preferentially binds to ETBR1 over ETBR2.

8. The method of claim 7, wherein upon administration, the lasso peptide specifically inhibits ETBR1.

9. The method of any one of claims 1 to 8, wherein upon administration, the lasso peptide
(a) inhibits ETBR-mediated signaling pathway; and/or
(b) downregulates ETBR expression on the surface of the neoplastic cells and/or endothelial cells in the microenvironment of the neoplastic cells.

10. The method of claim 9, wherein inhibition of the ETBR-mediated signaling pathway is measured by
(a) inhibition of release of relaxing factors;
(b) upregulation of intercellular adhesion molecule-1 (ICAM-1) expression and clustering;
(c) increase in migration of intraepithelial tumor infiltrating leukocytes (TILs) into the microenvironment of the neoplastic cells;
(d) inhibition of angiogenesis in the microenvironment of neoplastic cells;
(e) inhibition of growth and/or metastasis of neoplastic cells; and/or
(f) increase in apoptosis of neoplastic cells.

11. The method of claim 10, wherein the relaxing factors are nitric oxide, prostacyclin, and endothelium-derived hyperpolarizing factor, Ca²⁺, protein kinase C, mitogen-activated protein kinase, or any combination thereof.

12. The method of claim 10, wherein the TILs comprises neutrophils, T cells, B cells, NK cells, monocytes or a combination thereof.

13. The method of claim 12, wherein the monocytes comprise macrophages and/or dendritic cells.

14. The method of any one of claims 1 to 13, wherein the neoplastic cells express ETBR.

15. The method of any one of claims 1 to 14, wherein the subject express ETBR in endothelial cells of vasculature in the microenvironment of the neoplastic cells.

16. The method of claim 14 or 15, wherein the ETBR is ETBR1 and/or ETBR2

17. The method of any one of claims 1 to 16, wherein proliferative disease is cancer.

18. The method of claim 17, wherein the cancer is breast cancer, pancreatic cancer, hepatocellular cancer, prostate cancer, ovarian cancer, gastric cancer, glioblastoma, melanoma, cancer of the head and neck, colorectal cancer, bladder cancer, vulvar cancer, esophageal squamous cell carcinoma, renal cancer, cervical cancer, salivary gland carcinoma, lung cancer, multiple myeloma, or Kaposi's sarcoma.

19. The method of claim 18, wherein the cancer is melanoma or ovarian cancer.

20. The method of claim 9, wherein the maximal percent inhibition of the ETBR-mediated signaling pathway is at least about 10%, 20%, 30%, 40%, 45%, 50%, 55%, 60%, 65%, 70%, 75%, 80%, 85%, 90%, 95%, or 100%.

21. The method of claim 9, wherein the maximal percent downregulation of ETBR expression is at least about 10%, 20%, 30%, 40%, 45%, 50%, 55%, 60%, 65%, 70%, 75%, 80%, 85%, 90%, 95%, or 100%.

22. The method of any one of claims 1 to 21, wherein the lasso peptide is conjugated to an agent.

23. The method of claim 22, wherein the agent is selected from the group consisting of a radioisotope, a metal chelator, an enzyme, a protein, a peptide, an antibody, a fluorescent compound, a bioluminescent compound, and a chemiluminescent compound.

24. The method of any one of claims 1 to 23, further comprising co-administering to the subject a second therapeutic agent with the lasso peptide.

25. The method of claim 24, wherein the second therapeutic agent is conjugated with the lasso peptide.

26. The method of claim 24 or 25, wherein the second therapeutic agent is an immunotherapy or chemotherapy.

27. The method of claim 24 or 25, wherein the immunotherapy is an anti-cancer vaccine or an immune checkpoint modulator.

28. A method of cell-free biosynthesis of a lasso peptide, comprising
contacting a peptide comprising a sequence selected from SEQ ID NOS:1-34 and 42-71 with a lasso peptide biosynthesis component in a cell-free biosynthesis reaction mixture; and
incubating the cell-free biosynthesis reaction mixture under a condition suitable for lasso formation to produce the lasso peptide.

29. A method of cell-free biosynthesis of a lasso peptide having an amino acid sequence selected from SEQ ID NOS:1-17 and 42-56, wherein the method comprises
contacting a peptide comprising a leader sequence and a lasso core peptide sequence that is selected from SEQ ID NOS:1-17 and 42-56 with a lasso peptide biosynthesis component in a cell-free biosynthesis reaction mixture; and
incubating the cell-free biosynthesis reaction mixture under a condition suitable for lasso formation to produce the lasso peptide;
wherein the lasso peptide biosynthesis component comprises a lasso peptidase capable of catalyzing removal of the leader sequence.

30. The method of claim 28 or 29, wherein the contacting comprises adding a first nucleic acid sequence encoding the peptide into the cell-free biosynthesis reaction mixture, and wherein the cell-free biosynthesis reaction mixture comprises in vitro TX-TL machinery configured to express the peptide.

31. The method of any one of claims 28-30, wherein the contacting comprises adding a second nucleic acid sequence encoding the lasso peptide biosynthesis component to the cell-free biosynthesis reaction mixture, and wherein the cell-free biosynthesis reaction mixture comprises in vitro TX-TL machinery configured to express the lasso peptide biosynthesis component.

32. The method of claim 31, wherein the lasso peptide biosynthesis component comprises a lasso cyclase.

33. The method of claim 31, wherein the lasso peptide biosynthesis component comprises a lasso peptidase and a lasso cyclase, and wherein the contacting comprises adding the second nucleic acid sequence encoding the lasso cyclase and a third nucleic acid sequence encoding the lasso peptidase.

34. The method of claim 31, wherein the lasso peptide biosynthesis component comprises a lasso cyclase and a post-translationally modified peptide (RiPP) recognition element (RRE), and wherein the contacting comprises adding the second nucleic acid sequence encoding the lasso cyclase and a fourth nucleic acid sequence encoding the RRE.

35. The method of claim 31, wherein the lasso peptide biosynthesis component comprises a lasso peptidase, a lasso cyclase and a post-translationally modified peptide (RiPP) recognition element (RRE), and wherein the contacting comprises adding the second nucleic acid sequence encoding the lasso cyclase, a third nucleic acid sequence encoding the lasso peptidase and a fourth nucleic acid sequence encoding the RRE.

36. The method of any one of claims 31 to 35, wherein at least two of the first, second, third and fourth nucleic acid sequences are in a same nucleic acid molecule.

37. The method of any one of claims 28 to 36, wherein the cell-free biosynthesis reaction mixture comprises cell extract or supplemented cell extract.

38. A method for producing a lasso peptide using a non-naturally occurring microbial organism, wherein the method comprises
introducing into the microbial organism a first nucleic acid sequence encoding a peptide comprising a sequence of SEQ ID NOS:1-34 and 42-71 and a second nucleic acid sequence encoding a lasso peptide biosynthesis component;
culturing the microbial organism under a condition suitable for lasso formation to produce the lasso peptide.

39. A method for producing a lasso peptide having an amino acid sequence selected from SEQ ID NOS:1-17 and 42-56, wherein the method comprises
introducing into the microbial organism a first nucleic acid sequence encoding a peptide comprising a leader sequence and a lasso core peptide sequence that is selected from SEQ ID NOS:1-17 and 42-56 and a second nucleic acid sequence encoding a lasso peptide biosynthesis component; and
culturing the microbial organism under a condition suitable for lasso formation to produce the lasso peptide;
wherein the lasso peptide biosynthesis component comprises a lasso peptidase capable of catalyzing removal of the leader sequence.

40. The method of claim 38 or 39, wherein the lasso peptide biosynthesis component comprises a lasso cyclase.

41. The method of claim 38 or 39, wherein the lasso peptide biosynthesis component comprises a lasso peptidase and a lasso cyclase, and wherein the method comprises introducing the second nucleic acid sequence encoding the lasso cyclase and a third nucleic acid sequence encoding the lasso peptidase.

42. The method of claim 38 or 39, wherein the lasso peptide biosynthesis component comprises a lasso cyclase and a post-translationally modified peptide (RiPP) recognition element (RRE); and wherein the method comprises introducing the second nucleic acid sequence encoding the lasso cyclase and a fourth nucleic acid sequence encoding the RRE.

43. The method of claim 38 or 39, wherein the lasso peptide biosynthesis component comprises a lasso peptidase, a lasso cyclase and a post-translationally modified peptide (RiPP) recognition element (RRE); and wherein the method comprises introducing the second nucleic acid sequence encoding the lasso cyclase, a third nucleic acid sequence encoding the lasso peptidase, and a fourth nucleic acid sequence encoding the RRE.

44. The method of any one of claims 38 to 43, wherein at least two of the first, second, third and fourth nucleic acid sequences are in a same nucleic acid molecule.

45. The method of any one of claims 38 to 44, wherein the microbial organism is E. coli, Vibrio natriegens, Burholderia spp Corynebacterium glutamicum, or Sphingomaons subterranean.

46. The method of any one of claims 38 to 45, wherein the culturing is performed under a substantially anaerobic condition.

47. The method of claim 28 or 38, wherein the peptide comprises an amino acid sequence selected from SEQ ID NOS:1-17 and 42-56 and wherein the lasso peptide biosynthesis component comprises a lasso cyclase.

48. The method of claim 47, wherein the lasso peptide biosynthesis component further comprises a lasso peptidase and/or a RRE.

49. The method of claim 28 or 38, wherein the peptide comprises an amino acid sequence selected from SEQ ID NOS:18-34 and 57-71 and wherein the lasso peptide biosynthesis component comprises a lasso peptidase and a lasso cyclase.

50. The method of claim 49, wherein the lasso peptide biosynthesis component further comprises a RRE.

51. The method of any one of claims 32 to 35 and claims 40 to 43, wherein the lasso cyclase comprises the sequence of SEQ ID NO:36.

52. The method of any one of claims 33, 35, 41 and 43, wherein the lasso peptidase comprises the sequence of SEQ ID NO:35

53. The method of any one of claims 34, 35, 42 and 43, wherein the RRE comprises the sequence of SEQ ID NO:37

54. The method of any one of claims 28 to 53, wherein the lasso peptide comprises an amino acid sequence selected from SEQ ID NOS:1-17 and 42-56, and wherein the lasso peptide is G1-D9 cyclized.

55. The method of any one of claims 28 to 54, wherein the method further comprises isolating the lasso peptide from the cell-free biosynthesis reaction mixture of the culture medium of the microbial organism.

56. A biosynthesized lasso peptide produced by the method of any one of the claims 28 to 55.

57. A pharmaceutical composition comprising the biosynthesized lasso peptide of claim 56 and a pharmaceutically acceptable carrier.

58. The pharmaceutical composition of claim 57, wherein the composition further comprises a second therapeutic agent for managing, preventing or treating cancer.

59. The pharmaceutical composition of claim 58, wherein the second therapeutic agent is chemotherapy or immunotherapy for cancer.

60. The pharmaceutical composition of claim 59, wherein the second therapeutic agent is an anti-cancer vaccine or immune checkpoint modulator.

61. A method for managing, preventing or treating an endothelin-B medicated proliferative disease in a subject, comprising administering to the subject a pharmaceutically effective amount of the biosynthesized lasso peptide of claim 56 or the pharmaceutical composition of any one of claims 57 to 60.
